SEQUENCE LISTING (1 sequence)

SEQ ID NO: 1 - protein, 197 aa, Homo sapiens
MSRLSRSLLWAATCLGVLCVLSADKNTTQHPNVTTLAPISNVTSAPVTSLPLVTTPAPETCEGRNSCVSCFNVSVVNTTCFWIECKDESYCSHNSTVSDCQVGNTTDFCSVSTATPVPTANSTAKPTVQPSPSTTSKTVTTSGTTNNTVTPTSQPVRKSTFDAASFIGGIVLVLGVQAVIFFLYKFCKSKERNYHTL

(19) United States
(12) Patent Application Publication
(10) Pub. No.: US 2021/0309970 A1
(43) Pub. Date: Oct. 7, 2021

(54) COMPOSITIONS AND METHODS FOR CELL TRANSPLANTATION

(71) Applicants: THE CHILDREN'S MEDICAL CENTER CORPORATION, Boston, MA (US); DANA-FARBER CANCER INSTITUTE, INC., Boston, MA (US)

(72) Inventors: LUCA BIASCO, BOSTON, MA (US); MARIANA LOPERFIDO, BOSTON, MA (US); CRISTINA BARICORDI, BOSTON, MA (US); DANILO PELLIN, BOSTON, MA (US)

(73) Assignees: THE CHILDREN'S MEDICAL CENTER CORPORATION, BOSTON, MA (US); DANA-FARBER CANCER INSTITUTE, INC., BOSTON, MA (US)

(21) Appl. No.: 17/213,417

(22) Filed: Mar. 26, 2021

Related U.S. Application Data
(63) Continuation of application No. PCT/US2019/053232, filed on Sep. 26, 2019.
(60) Provisional application No. 62/737,483, filed on Sep. 27, 2018.

(51) Int. Cl. C12N 5/0789; G01N 33/569; A61K 35/28

(52) U.S. Cl. CPC C12N 5/0647; G01N 33/56966; C12N 2510/00; C12N 2501/599; G01N 2333/70596; A61K 35/28

(57) ABSTRACT

The invention features methods of identifying a hematopoietic/stem progenitor population for clinical transplantation and gene therapy, and compositions for transplantation or gene therapy featuring cells characterized as CD34+CD164^{High}.

### CROSS-REFERENCE TO RELATED APPLICATION

This application is a continuation patent application of International Application No. PCT/US2019/053232, filed Sep. 26, 2019, which claims the benefit of and priority to U.S. Provisional Application No. 62/737,483, filed Sep. 27, 2018, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Oct. 17, 2019, is named 167705_016601_PCT_SL.txt and is 2,102 bytes in size.

### BACKGROUND OF THE INVENTION

Human hematopoietic stem/progenitor cells (HSPCs) are commonly identified by expression of the antigen CD34. CD34⁺ cells are heterogeneous, and there are ongoing efforts to classify their substructure by immunophenotyping, and according to their differentiation and in vivo survival potential. The CD34⁺ cell population structure is unresolved, with recent studies showing that the current immunophenotypically-defined CD34⁺ subsets could be more heterogeneous than previously thought. A possible reason for the lack of resolution is that enrichment methods for CD34⁺ cells may bias the representation of cell states during early hematopoietic commitment, as the CD34 marker is downregulated at different rates along commitment to different cell fates. Improved methods of identifying cells appropriate for transplantation are required.

### SUMMARY OF THE INVENTION

As described below, the present invention features methods of identifying a hematopoietic stem/progenitor cell population for clinical transplantation and gene therapy, and compositions for transplantation featuring cells characterized as CD34+CD164^{High}.

In one aspect, the invention generally provides a method for obtaining an enriched population comprising primitive hematopoietic stem/progenitor cells for use in transplantation or gene therapy, the method involving selecting one or more CD34⁺CD164^{high} cells, and expanding said cells in culture to enrich for stem/progenitor cells.

In another aspect, the invention provides a method for selecting early versus late hematopoietic stem/progenitor cells, the method involving isolating CD34⁺CD164^{high} cells from CD34⁺CD164^{low} cells.

In another aspect, the invention provides a method for obtaining an enriched population comprising primitive hematopoietic stem/progenitor cells for use in transplantation or gene therapy, the method involving selecting one or more CD34⁺CD164^{high} cells; expanding said cells in culture to obtain a population of stem cells; and selecting CD34⁺CD164^{high} cells from the population of, thereby obtaining a population of primitive hematopoietic stem/progenitor cells. In one embodiment, greater than about 60% (e.g., 70, 80, 90, 95% or more) of the cells present in the population are CD34⁺CD164^{high} cells.

In another aspect, the invention provides a cell or population of cells obtained according to the method of any one of the previous aspects or any other aspect of the invention delineated herein. In one embodiment, the cell contains a mammalian expression vector encoding a recombinant protein.

In another aspect, the invention provides a method for treating a subject in need of an increase in hematopoietic stem/progenitor cells, the method comprising administering to the subject an effective amount of a cell of a previous aspect present in a pharmaceutically acceptable excipient.

In another aspect, the invention provides a method for expressing a therapeutic gene in a hematopoietic cell of a subject, involving contacting a hematopoietic stem/progenitor cell with a recombinant vector comprising a nucleic acid sequence encoding a therapeutic or detectable polypeptide to obtain a transgenic cell transduced with the vector; and administering the cell to a subject, such that the transgenic cell or a progeny cell thereof populates bone marrow in the subject and expresses the therapeutic or detectable polypeptide.

In another aspect, the invention provides a method to support short-term granulopoiesis in conditioned neutropenic patients, the method comprising administering to the subject an effective amount of a cell of a previous aspect present in a pharmaceutically acceptable excipient.

In another aspect, the invention provides a pharmaceutical composition containing an effective amount of a cell (e.g., a cell selected as CD34⁺CD164^{high}) of a previous aspect.

In another aspect, the invention provides a kit for treating a subject in need of an increase in hematopoietic stem/progenitor cells, the kit comprising a cell (e.g., a cell selected as CD34+CD164^{high}) and instructions for administering the cell to a subject (e.g., human).

In various embodiments of any of the above aspects or any other aspect of the invention delineated herein, the CD34⁺CD164^{high} selection enriches for stem/progenitor cells at greater than about 60% efficiency. In other embodiments, the CD34⁺CD164^{high} selection enriches for stem/progenitor cells at greater than about 70%, 80% or 90% efficiency. In various embodiments of any of the above aspects or any other aspect of the invention delineated herein, the method further involves characterizing the expanded population for the presence of early stage progenitor cells by detecting increased levels of CD164 versus the level of CD164 present in a late progenitor cell. In various embodiments of any of the above aspects or any other aspect of the invention delineated herein, there is an order of magnitude difference in the level of CD164 present in an early stage progenitor cell (e.g., at least about 10³ to 10⁴) versus the level present in a late progenitor cell (e.g., about 102). In various embodiments of any of the above aspects or any other aspect of the invention delineated herein, the level of CD164 is at least about 10³ to 10⁴ whereas the level of CD164 is about 10² in a late stage progenitor cell. In various embodiments of any of the above aspects or any other aspect of the invention delineated herein, the selection is by magnetic enrichment. In various embodiments of any of the above aspects or any other aspect of the invention delineated herein, the method excludes B cell progenitors (e.g., expressing CD79a and/or CD10). In various embodiments of any of the above aspects or any other aspect of the invention delineated herein, the selecting involves contacting the cell with a CD34 antibody and a CD164 antibody. In various embodiments of any of the above aspects or any other aspect of the invention delineated herein, each antibody is fixed to a substrate (e.g., a magnetic bead). In various embodiments of any of the above aspects or any other aspect of the invention delineated herein, the selecting is by fluorescence activated cell sorting. In various embodiments of any of the above aspects or any other aspect of the invention delineated herein, the methods involve characterizing the cells for one or more markers selected from the group consisting of CD3, CD7, CD10, CD14, CD16, CD15, CD19, CD20, CD38, CD41, CD45RA, CD56, CD71, CD90, CD135, and Lin. In various embodiments of any of the above aspects or any other aspect of the invention delineated herein, the administration is by bone marrow transplant or a hematopoietic stem cell transplant. In various embodiments of any of the above aspects or any other aspect of the invention delineated herein, a cell (e.g., a cell selected as CD34⁺CD164^{high}) is administered parenterally. In various embodiments of any of the above aspects or any other aspect of the invention delineated herein, the cell is derived from a donor subject. In particular embodiments, the donor subject and the host subject are the same individual. In particular embodiments, the subject is undergoing radiation and/or chemotherapy. In particular embodiments, the subject has a condition (e.g., lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation, immune thrombocytopenic purpura, myelodysplasia, thrombocytotic disease, thrombocytosis, congenital neutropenias, myelodysplastic syndrome, and neutropenia associated with chemotherapy and/or radiotherapy). In various embodiments of any of the above aspects or any other aspect of the invention delineated herein, the subject is undergoing chemotherapy and/or radiotherapy for myeloma, non-Hodgkin's lymphoma, Hodgkin's lymphoma, or leukemia.

In another aspect, a method is provided for obtaining an enriched population that includes cells having basophilic potential for use in transplantation or gene therapy, the method involves selecting one or more Lin−CD34+CD135− cells, and expanding said cells in culture to enrich for cells having basophilic potential. Another aspect of the present invention is a cell or population of cells obtained according to this method.

A method is also provided in another aspect for obtaining an enriched population that includes cells having basophilic potential for use in transplantation or gene therapy, the method involves (a) selecting one or more having basophilic potential cells, (b) expanding said cells in culture to obtain a population of stem cells, and (c) selecting Lin−CD34+CD135− cells from the population of step (b), thereby obtaining a population of cells having basophilic potential. In some embodiments, greater than about 60% of the cells present in the population of step (b) are Lin−CD34+CD135−. In an embodiment, a cell or population of cells is obtained according to this method. In another aspect, this cell or population of cells are used in a method for treating a subject in need of an increase in basophils that involves administering to the subject an effective amount of the cell or population of cells present in a pharmaceutically acceptable excipient.

In another aspect, a method is provided for treating a subject in need of an increase in basophils, the method involves administering to the subject an effective amount of a cell or population of cells present in a pharmaceutically acceptable excipient.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound.

By “allogeneic” is meant cells of the same species.

By “antibody” is meant any immunoglobulin polypeptide, or fragment thereof, having immunogen binding ability.

By “ameliorate” is meant decrease, suppress, attenuate, diminish, arrest, or stabilize the

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “autologous” is meant cells from the same subject.

“Basophilic potential” refers to tendency of a cell or population of cells to differentiate into basophils. For example, Lin−CD34+CD135+ cells have high basophilic potential as they give rise to basophils with high efficiency.

By “bone marrow derived cell” is meant any cell type that naturally occurs in bone marrow.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “CD164 protein” is meant a protein that responds with an anti-CD164 antibody having at least about 85% identity to UniProt. Accession No. Q04900, which is reproduced below:

sp|Q04900|MUC24_HUMAN Sialomucin core protein 24
OS = Homo sapiens OX = 9606 GN = CD164 PE = 1
SV = 2
(SEQ ID NO: 1)
MSRLSRSLLWAATCLGVLCVLSADKNTTQHPNVTTLAPISNVTSAPVTSL
PLVTTPAPETCEGRNSCVSCFNVSVVNTTCFWIECKDESYCSHNSTVSDC
QVGNTTDFCSVSTATPVPTANSTAKPTVQPSPSTTSKTVTTSGTTNNTVT
PTSQPVRKSTFDAASFIGGIVLVLGVQAVIFFLYKFCKSKERNYHTL

Other CD164 proteins are described, for example, at Ref. Seq. NP_001135873, NP_001135874, NP_001135875, NP_001135876, and NP_001333429.

By “CD34⁺CD164^{high} cell” is meant a cell having detectable CD34 and having increased levels of CD164 relative to a CD34-expressing reference cell.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include diseases ameliorated by bone marrow transplant or hematopoietic stem cell transplant, including cancer, e.g., cancers being treated by chemo and/or radiation therapy, including myeloma, non-Hodgkin's lymphoma, Hodgkin's lymphoma, or leukemia.

By “effective amount” is meant the amount of a cellular composition of the invention required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “engraft” as used herein refers to the process of stem cell incorporation into a tissue of interest in vivo through contact with existing cells of the tissue.

“Hematopoietic stem/progenitor cells” (or hematopoietic stem cells (HSCs)) as used herein refer to immature blood cells having the capacity to self-renew and to differentiate into the more mature blood cells (also described herein as “progeny”) comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). Hematopoietic progenitor cells are interchangeably described as “hematopoietic stem cells” throughout the specification. It is known in the art that such cells may or may not include CD34+ cells. CD34+ cells are immature cells present in the “blood products” described below, express the CD34 cell surface marker, and are believed to include a subpopulation of cells with the “progenitor cell” properties defined above. In particular embodiments, cells of the invention are characterized for the presence and/or level of CD164. CD164 is used alone or in combination with CD34, CD38, and CD90. Human HSCs have been defined with respect to staining for Lin39, CD34, CD38, CD43, CD45RO, CD45RA, CD59, CD90, CD109, CD117, CD133, CD166 and HLA DR (human).

It is well known in the art that hematopoietic progenitor cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by high performance liquid chromatography (HPLC) analysis.

By “marker” is meant any protein or polynucleotide having an alteration in expression level or activity that is indicative of cell fate, cell differentiation, or developmental potential. In one embodiment, CD164 is a marker used to define a primitive population of HSCs for delivery to a subject.

As used herein, “obtaining” as in “obtaining an agent” includes synthesizing, purchasing, or otherwise acquiring the agent.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

The term “stem cell” is meant a multipotent or pluripotent cell having the capacity to self-renew and to differentiate into multiple cell lineages.

By “stem cell generation” is meant any biological process that gives rise to stem cells. Such processes include the differentiation or proliferation of a stem cell progenitor or stem cell self-renewal.

By “subject” is meant a mammal, including, but not limited to, a human or non-human mammal, such as a bovine, equine, canine, ovine, or feline.

By “syngeneic,” as used herein, refers to cells of a different subject that are genetically identical to the cell in comparison.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1-1A to 1-1H provide an experimental workflow and transcriptional map for human HSPCs. FIG. 1-1A provides a schematic for experimental design and workflow of data analysis. Two experiments have been performed on two separated healthy donors to generate two single-cell transcriptome maps. FIG. 1-1B shows a gating strategy used for the FACS-sorting of seven HSPC subsets from magnetic-beads purified CD34+ cells of a healthy donor blood marrow (BM) (HSC, Hematopoietic Stem Cells; MPP, MultiPotent Progenitors; MLP, MultiLymphoid Progenitors; PreB/NK, Pre-B lymphocytes/Natural Killer cells; MEP, Megakaryocyte-Erythroid Progenitors; CMP, Common Myeloid Progenitors; GMP, Granulocyte-Monocyte Progenitors). FIG. 1-1C provides a SPRING plot of the seven HSPCs single-cell transcriptomes. Each point is one cell. Labels at the edges represent the transcriptional states associated to early lineage commitment (Meg, Megakaryocytes; E, Erythroid cells; G, Granulocytes; DC, Dendritic Cells; Ly1/Ly2, Lymphoid B, T, NK cells). The same shading code as in FIG. 1-1B has been used to identify HSPC subsets. FIG. 1-1D shows representative gene expression maps of lineage defining genes (PLEK, Meg; HBB, E; MPO, G; SPIB, DC; CD79A and DNTT, Ly1/2). FIG. 1-1E shows the classification of individual cells into homogenous transcriptional groups numbered from 1 to 11, based on inferred principal trajectories (FIG. 3-2A for details). FIG. 1-1F shows the predicted hierarchy based on two steps PBA. FIG. 1-1G provides a heatmap showing the expression average in groups shown in FIG. 1-1E for statistically significant genes coding for CD markers (LRT adjusted p-value <0.05). FIG. 1-1H provides gene expression maps of CD34 and CD164.

FIG. 1-2A and FIG. 1-2B show the observed and predicted cell density estimations by means of nonparametric kernel method. FIG. 1-2A is a transcriptome map showing observed (left) and predicted (right) cell density estimations of sorted HSPCs. FIG. 1-2B is a transcriptome map showing observed (left) and predicted (right) cell density estimations of sorted Lin−CD34/CD164 cells.

FIG. 2-1A to FIG. 2-1F show a human LIN-compartment investigation by means of Lin−CD34/CD164 fractionating. FIG. 2-1A shows a gating strategy for the FACS-sorting of four novel subsets inside the Lin− fraction of a healthy donor BM, according to CD34 and CD164 expression (left panels). Relative contribution of CD71+ progenitors is shown on the right panels. FIG. 2-1B shows a SPRING plot of the four Lin−CD34/CD164 subsets single-cell transcriptomes. Each point is one cell. Labels at the edges represent the transcriptional states associated to early lineage commitment (P, early Progenitor cells; Meg, Megakaryocytes; E, Erythroid cells; BEM, Basophils/Eosinophils/Mast-cells progenitors; N, Neutrophils; M, Monocytes; DC, Dendritic Cells; Ly, Lymphoid TB/NK cells). Gene expression maps are available in FIG. 4-2. FIG. 2-1C shows predicted hierarchy based on two steps PBA. FIG. 2-1D shows the classification of individual cells into homogenous transcriptional groups numbered from 1 to 15, based on inferred principal trajectories. Solid lines show results based on final converged iteration. Dashed lines added manually to highlight a potential additional trajectory not present in final iteration and inferred by visual inspection (DC-M). FIG. 2-1E show gene dynamics associated to branching and fate decisions. Plots on the left, branching and groups; Mirror heatmaps, expression of statistically significant genes differentially expressed along each branch pseudotime (LRT adjusted p-value <0.05); Plots on the right, a selection of three transcription factors differentially expressed along each branch (LRT adjusted p-value <0.05). FIG. 2-1F shows a projection of the transcriptional states of the seven HSPCs onto the Lin−CD34/CD164 map.

FIG. 2-2A and FIG. 2-2B show a visualization of the sorted subpopulations on the SPRING graphs. The FACS-sorted cell fractions have been individually highlighted in orange on the corresponding SPRING graphs. FIG. 2-2A show the seven sorted HSPC subpopulations. FIG. 2-2B show the four fractions isolated in Lin−CD34/CD164 cells.

FIG. 3-1A to FIG. 3-1C show human Lin−CD34/CD164 versus mouse Kit+ transcriptome map and gene expression dynamics analysis. FIG. 3-1A shows the classification of individual cells into 11 homogenous transcriptional groups, based on inferred principal trajectories on mouse Kit+ transcriptome data. Group labels and colors have been set to highlight similarities with Lin−CD34/CD164 fractioning map. Solid lines show results based on final converged iteration. Dashed lines added manually to highlight a potential additional trajectory not present in final iteration and suggested by PBA analysis reported in the middle (DC-M). FIG. 3-1B provides a comparison of human and mouse transcriptional states during erythropoiesis. Upper panels, schemes of the comparison. Mirror heatmaps, expression of the 721 orthologous genes selectively expressed along the human and mouse erythroid differentiation (LRT adjusted p-value <0.05). FIG. 3-1C shows representative comparable dynamics of the orthologues TRIB2/Trib2 and CA2/Car2 versus divergent dynamics of the orthologues CD47/Cd47 and ZFPM1/Zfpm1.

FIG. 3-2A to 3-2C shows transcriptional principal trajectories identification procedures. Graphical representation of key intermediate steps underlying the estimation of principal trajectories for the transcriptomes of FIG. 3-2A sorted HSPCs, FIG. 3-2B sorted Lin−CD34/CD164 cells and FIG. 3-2C mouse Kit+ cells. Graphs showing Iteration:0: consolidation points initialization; Iteration:2: and Iteration:10: consolidation points distribution after 2 and 10 iterations; Final iteration: estimated consolidation points distribution returned by structure-aware filtering algorithm; Merging: consolidation point set reduction by iterative merging; MST: branching reconstruction by Minimum Spanning Tree; Principal trajectories: segmentation of reconstructed skeleton; Cells grouping: cells-branch association.

FIGS. 4-1A to 4-1K show immunophenotyping and in vitro functional assays of CD164 expressing subsets in BM CD34+ cells. FIG. 4-1A provides experimental design. Purified CD34+ cells from BM were characterized for the expression of CD164 together with the classical HSPCs markers by flow cytometry. CD164^{high} and CD164^{low} cell fractions were sorted from CD34+ cells and their lineage potential was investigated through in vitro functional assays. CD34+ cells were employed as control. FIGS. 4-1B to 4-1E shows immunophenotyping. FIG. 4-1B comprises representative FACS plots showing the contribution of Lin−/+ cells and HSPC subsets in CD164^{high} and CD164^{low} fractions of CD34+ cells. FIG. 4-1C is a graph showing the percentage of CD164^{high} and CD164^{low} fractions in CD34+ cells. Shown are mean±s.d. from three independent BM. FIG. 4-1D comprises bar graphs showing the content of Lin−/+, CD38−, CD90+ cells and HSPCs in CD164^{high} and CD164^{low} fractions. Values are mean±s.d. from three independent BM. Unpaired two-tailed t-test (*P<0.5, **P<0.005). For the HSPCs bar graph, the method of moment estimations of each HSPC subpopulation proportion in CD164^{high}versus CD164^{low} fractions are provided in Table 3. FIG. 4-1E is a pie-chart distribution of CD164^{high} and CD164^{low} fractions on HSPC subsets from nine independent BM. FIG. 4-1F comprises a sorting gating strategy (CFCs, colony-forming cells, BFU-E, burst-forming unit-erythroid cells, CFU-E, colony-forming unit-erythroid cells, CFU-GM, colony-forming unit-granulocyte/macrophages) and bar graphs showing the total number (left) and type of colonies (right) scored at day 14 in a methylcellulose-based colony-forming unit (CFU) assay. Shown are mean±SD from six independent BM. Statistics by independent samples, heteroscedastic, two-tailed Student's t test (*p<0.05). FIG. 4-1G comprises growth curves from three different culture conditions. Mk, Megakaryocyte; My, Myeloid. Values are mean±SD from nine independent BM. Statistics by independent samples, heteroscedastic, two-tailed Student's t test (*p<0.05, **p<0.0005, ***p<0.0001). FIG. 4-1H comprises bar graphs summarizing single-cell (SC) assays and showing the total number of colonies obtained from each population in the Mk (left) and My (right) differentiating culture. Shown are median±error from three independent BM. Statistics by independent samples, two-tailed Student's t test (*p<0.01). FIG. 4-11 is a diagram of in vivo experimental design. Sorted CD164high and CD164low populations were transplanted in NBSGW mice each at the dose of 2.5×105 cells/mouse. In order to reflect the real proportions in the human BM, immunomagnetic-selected CD34+ cells were transplanted at the dose of 5.0×105 cells/mouse. The human engraftment was evaluated in the murine peripheral blood at different time points, and in BM and spleen at 16 weeks post-transplant. FIG. 4-1J comprises graphs showing human CD45+ cell engraftment in murine PB (left; CD164^{high}, n=3; CD164^{low}, n=3; CD34+, n=4 mice) and BM (right; CD164^{high}, n=3; CD164^{low}, n=2; CD34+, n=4 mice). FIG. 4-1K comprises bar graphs showing the relative contribution of human cell populations inside the hCD45+ and hCD45− compartments in murine BM. (CD164^{high}, n=3; CD164^{low}, n=2; CD34+, n=4 mice)

FIG. 4-2 shows gene expression maps for relevant genes. Lineage defining genes of the sorted Lin−CD34/CD164 cells: HLF, P; PLEK, Meg; HBB, E; CLC, BEMP; ELANE, N; SAMHD1, M; MPO, undifferentiated granulocytes; IRF8, DC; DNTT, Ly. Other genes: CD34; CD164

FIGS. 5A-5D show gene expression variation among branches and fate decision signatures in Lin−CD34/CD164 transcriptome. FIG. 5A provides a heatmap representation of gene expression levels among cells groups shown in FIG. 2D for significant genes (LRT adjusted p-value <0.05) known to code for CD markers. Individual gene expression data have been row normalized among groups during heatmap generation. FIG. 5B shows gene dynamics associated to branching and fate decisions. The following comparisons are shown: 4 versus 5, 7 versus 8 and 10 versus 13. Columns report respectively: branching and groups considered (left); heatmaps of expression regression curves for genes showing a statistically significant difference (central); three significant transcription factors (right). FIG. 5C is a heatmap for significant proto-oncogenes (LRT adjusted p-value <0.05) with documented activity relevant to blood cancer according to COSMIC catalogue among groups identified in sorted Lin−CD34/CD164 cells. FIG. 5D is a heatmap for significant proto-oncogenes (LRT adjusted p-value <0.05) with documented activity relevant to blood cancer according to COSMIC catalogue among groups identified in sorted HSPCs. FIG. 6 provides a projection of computationally identified groups from the HSPCs map onto the Lin−CD34/CD164 topology. Each cells group derived from principal trajectories identification analysis of the sorted HSPCs has been projected on Lin−CD34/CD164 topology. The labels of the seven FACS-sorted subpopulations have been added.

FIG. 7 focuses on low-or-negative correlated genes among human and mouse erythropoiesis. Mirror heatmaps representing estimated regression curves for 89 orthologous genes exhibiting a low-or-negative correlation (Pearson correlation <0.5). Performing enrichment analysis by means of the Reactome pathway database tool, the Translation pathway was found to be significantly over-represented (p-value: 5.01E-5). In the bottom dashed rectangle, a specific mirror heatmap for gene hits is shown.

FIGS. 8A and 8B shows a cytometric analysis of CD38 and CD164 expression in expansion culture. FIG. 8A comprise FACS plots showing the expression of CD164 with respect to CD34 in sorted CD34+CD164^{high} and CD34+CD164^{low} populations, at day 0 and day 4 in expansion culture. CD34+ cells were also analyzed. FIG. 8B comprises FACS plots showing the expression of CD38 with respect to CD34 in sorted CD34+CD164^{high} and CD34+CD164^{low} populations, at day 0 and day 4 in expansion culture. CD34+ cells were also analyzed. Shown are three independent BM. Left schemes represent the predicted path of differentiation from the most primitive fractions CD34^{high}CD164^{high} cells in FIG. 8A and CD34^{high}CD38− cells in FIG. 8B. Shown are also the schemes of the observed paths of differentiation.

FIGS. 9A-9E provides additional data on immunophenotyping and in vitro functional assays. FIG. 9A shows immunophenotyping. FACS plots showing the content of Lin−/+ cells and HSPC subsets in the CD164^{high} and CD164^{low} fractions of CD34+ cells from three independent BM. FIGS. 9B-9E are in vitro assays on sorted CD164^{high} and CD164^{low} populations, and on CD34+ cells. FIG. 9B shows fractions of CD34^{high}, CD34^{low} and CD34^{neg} at day 0 and in differentiation states at day 4 in explant (Exp) culture, or at day 14 in Mk and My culture conditions. FIG. 9C includes the percentage of Lin− and Lin+ cells at day 0 and day 4 in Exp culture. FIG. 9D is a bar graph showing the expression of lineage positive markers CD15 and CD19 in CD34+ and CD34− cell fractions at day 0, at day 4 in Exp culture and day 14 in My culture. FIG. 9E shows the percentage of CD41+CD71−GPA− cells at the end of the megakaryocyte differentiation culture. Values are mean±s.d from three independent BM. Unpaired two-tailed t-test (*P≤0.05).

FIGS. 10A-10C includes explanatory figures for structure-aware filtering algorithm, cell-branch association, cell progress ordering and gene expression analyses. FIG. 10A provides results of structure-aware filtering based on the iterative update of consolidation points positions according to a velocity field with two components. The pulling force (left) make consolidation points to move towards regions enriched for data points. The repulsion term (right) is the sum of all the pushing forces exerted by neighbor consolidation points and can only move points on a specific, locally optimal, line of action (first principal component). FIG. 10B shows each cell is associated to the closest branch by comparing cell-trajectories orthogonal distances (left). Cell pseudotime value, measuring the progression status along the differentiation process, is given by the rescaled distance between cell orthogonal projection onto the associated trajectory and branch stem point (right). FIG. 10C includes examples of the gene expression analyses performed. The left-most graph shows ANXA2 group-wise gene expression (adjusted p-value: 9.31E-159). The solid lines in each group's column correspond to each group's averages (M₁) and the dashed line corresponds to the overall mean (M₀) for all groups. The center graph provides graphical illustration of ANXA2 association with progression (pseudotime) along branch 12 (Monocytes) in Lin−CD34/CD164 transcriptome map (adjusted p-value: 1.0821E-126). The solid line represents the spline based regression curve (M₁), whereas dashed line represents the restricted model M₀. The right graph shows ANXA2 expression comparison among groups 11 (Neutrophils) and 12 (Monocytes). Group specific regression curves with common intercept (M₁) are shown with solid lines. The dashed line corresponds to the nonlinear model fitted without considering group labels (adjusted p-value: 1.3409E-260).

FIGS. 11A and 11B provide a diffusion map representation for Lin−CD34/CD164 dataset. FIG. 11A comprises two views on the 3-dimensional diffusion map calculated. FIG. 11B comprises diffusion map tips that have been labeled according to specific gene expression signatures. Diffusion components 1 and 2 (DC1, DC2) capture lympho-myeloid and erythroid heterogeneity, whereas DC3 describes baso-eosinophil differentiation progression.

FIGS. 12A-12C illustrate transcriptional principal trajectories identification procedure on diffusion map for Lin−CD34/CD164 dataset. FIG. 12A provide estimated consolidation points distribution returned by structure-aware filtering algorithm. FIG. 12B is a segmentation of reconstructed skeleton. FIG. 12C is a cells-branch association. The procedure identifies 17 segments in total. Inferred skeleton and groups 1-15 closely recapitulate the results obtained starting from SPRING topology shown in FIG. 2-1D.

FIGS. 13A-13H illustrate cell fate analyses of Lin−CD34+CD135− cells that support the MEP-associated origin of basophil progenitors. FIG. 13A is a projection of the transcriptional profile of cells belonging to group 9 in Lin−CD34/CD164 data set onto sorted HSPCs map. Pie chart on the bottom represents the immunophenotipic characteristic for HSPC cells identified as most similar. FIG. 13B is an experimental design showing sorting of Lin−CD34+CD135− and Lin−CD34+CD135+ populations from the BM CD34+ cells of three healthy donors. Their lineage potential was investigated through in vitro functional assays. FIG. 13C is a spatial distribution estimated by using a two-dimensional kernel density estimator for cell exhibiting: top graph, high expression (at RNA level) of FLT3 gene (normalized expression >0.9); bottom graph, no expression of FLT3 gene (normalized expression=0). FIG. 13D is a bar graph showing the content of HSPCs in CD135− and CD135+ fractions. Values are proportions estimates±SE, estimated using method of moments and Dirichlet-Multinomial model. Hypothesis testing has been performed by means of independent samples, heteroscedastic, two-tailed Student's t test. Details are provided in Table 2. FIG. 13E comprises growth curves from three different culture conditions. “My” denotes myeloid differentiating culture; “Mk” denotes megakaryocyte differentiation culture; “Baso” denotes basophil differentiation culture. Values are median±error. Statistics by independent samples, two-tailed Student's t test for each time point considered independently from the others (*p<0.05). FIG. 13F are graphs of single-cell (SC) assays showing the total number of colonies obtained from CD135− and CD135+ fractions at the end of the three different culture conditions. Shown are median±error. Statistics by independent samples, two-tailed Student's t test (*p<0.05). FIG. 13G comprises plots of FACS analysis of bona fide basophils (Baso) defined as CD14−CD15−FceRIA+CCR3+IL5RA+ cells on CD135− and CD135+ populations upon basophil (upper panel) and myeloid (lower panel) differentiation culture. FceRIA− pick indicates the negative control. FIG. 13H comprises bar graphs summarizing the cytometric analysis described in g. Shown are the percentage of Baso, CD14+ cells and CD15+ cells on CD135− and CD135+ populations from the basophil (left panel) and myeloid (right panel) differentiation culture. Values are median±error. Statistics by independent samples, two-tailed Student's t test (*p<0.05).

FIGS. 14A-14E illustrates characterization of basophils in human peripheral blood and upon in vitro differentiation. FIG. 14A comprises plots derived from a gating strategy developed for the identification and definition of basophils in the human peripheral blood. In the SSC-A low population, the fraction of CD14-CD15− cells was selected and investigated for the expression of FceRIA and CCR3. The double positive FceRIA+CCR3+ population has been defined as Basophils (Baso). This was confirmed by the IL-5RA expression in the Baso (darker pick's shift) with respect to the control FceRIA-− population (lighter pick). FIG. 14B comprises plots derived from cytometric analyses of CD34+ cells from 3 independent BM using the gating strategy described in FIG. 14A. FIG. 14C comprises plots derived from cytometric analysis of an isolated population of CD34+/Lin− cells. FIG. 14D comprises images of depicting giemsa staining of Lin−CD34+CD135− and Lin−CD34+CD135+ cells FACS-sorted from the 3 BM described in FIG. 14B and cultured in basophil (Baso) differentiation culture. Basophils and Monocytes are indicated respectively with orange and blue arrow heads. Scale bar, 20 FIG. 14E comprises FACS plots showing the presence of basophils mostly in CD135− cells but CD135+ cells from the 3 BM described in FIG. 14B upon differentiation in Baso differentiation culture (left panel). As control, the same cytometric analysis was performed upon My differentiation culture (right panel).

FIGS. 15A-15C depict immunophenotyping of G-CSF MPB CD34+ cells from 4 healthy donors. FIG. 15A comprises FACS plots showing CD164^{high} and CD164^{low} fractions in CD34+ cells from G-CSF MPB of 4 healthy donors. FIG. 15B is a bar graph showing the percentage of CD164^{high} and CD164^{low} fractions in MPB CD34+ cells. Values are Mean±SD. FIG. 15C comprises bar graphs showing the content respectively of Lin−/+, CD38−, CD90+ cells and HSPCs in CD164^{high} and CD164^{low} fractions, and in CD34+ cells. Values are Mean±SD. Statistics by Student t-test (*p<0.05; **p<0.01; ***p<0.005). For the HSPCs bar graph, statistics is provided in Table 4.

FIG. 16 comprises FACS plots showing CD164high and CD164low fractions in CD34+ cells from respectively BM (left) and plerixafor MPB (right) of the same sickle cell disease patient.

FIGS. 17A-17G illustrate in vitro functional assays of CD164 versus CD90 subsets FACS-sorted from BM CD34+ cells of 3 additional healthy donors. FIG. 17A is a gating strategy used to FACS-sort respectively CD164high/low fractions and CD90+/− fractions from BM CD34+ cells of 3 independent healthy donors (left) and bar graphs showing the percentage of purity of each sorted population (right). FIG. 17B comprises bar graphs showing the percentage of CD164high/low fractions (left) and CD90+/− fractions (right) in CD34+ cells. Shown are Median±Error. FIG. 17C is a bar graph showing the total number of CFCs scored at day 14 in a colony-forming assay. FIG. 17D is a bar graph showing the types of CFCs scored at day 14 in a colony-forming assay (CFCs, Colony Forming Cells, BFU-E, Burst-Forming Unit-Erythroid cells, CFU-E, Colony-Forming Unit-Erythroid cells, CFU-GM, Colony-Forming Unit-Granulocyte/Macrophages). Shown are Median±Error. FIG. 17E is a bar graph depicting the growth rate of the sorted subsets and CD34+ cells in expansion medium. FIG. 17F is a bar graph depicting the growth rate of the sorted subsets and CD34+ cells in myeloid (My) differentiation medium. FIG. 17G is a bar graph depicting the growth rate of the sorted subsets and CD34+ cells in megakaryocyte (MK) differentiation medium. Values are Median±Error.

FIGS. 18A-18C illustrate immunophenotypic profile of CD164 versus CD90 subsets upon in vitro functional assays of 3 additional healthy donors. FIG. 18A is a bar graph depicting the percentage of CD34^{high}, CD34^{low} and CD34^{neg} at day 0 and in differentiation states at day 4 in expansion (Exp) medium, or at day 14 in megakaryocyte (Mk) and myeloid (My) culture conditions of the sorted CD164^{high/low} fractions and CD90+/− fractions, and CD34+ cells. FIG. 18B is a bar graph showing the expression of lineage positive markers CD15 and CD19 in CD34+ and CD34− cell fractions at day 0, at day 4 in Exp culture and day 14 in My culture. FIG. 18C is a bar graph showing the percentage of CD41+CD71−GPA− cells normalized to the number of cells at the end of the megakaryocyte differentiation culture. Values are Median±Error from 3 independent BM. Statistics by Student t-test (*p<0.05).

FIG. 19 comprises FACS plots showing the expression of CD164 with respect to CD34 in sorted CD164^{high/low} fractions and CD90+/− fractions, and CD34+ cells at day 0 and day 4 in expansion culture. Shown are 3 independent BM. Left scheme represents the predicted path of differentiation from the most primitive fraction of CD34^{high}CD164^{high} cells. The observed path of differentiation (right scheme) perfectly overlaps.

FIG. 20 comprises FACS plots showing the expression of CD38 with respect to CD34 in sorted CD164^{high/low} fractions and CD90+/− fractions, and CD34+ cells at day 0 and day 4 in expansion culture. Shown are 3 independent BM. Left scheme represents the predicted path of differentiation from the most primitive fraction of CD34^{high}CD38^{neg} cells. The observed path of differentiation (right scheme) does not overlap.

FIG. 21 comprises FACS plots showing the expression of CD90 with respect to CD34 in sorted CD164^{high/low} fractions and CD90+/− fractions, and CD34+ cells at day 0 and day 4 in expansion culture. Shown are 3 independent BM. Left scheme represents the predicted path of differentiation from the most primitive fraction of CD34^{high}CD90^{pos} cells. The observed path of differentiation (right scheme) does not overlap.

FIGS. 22A and 22B depict the path of differentiation of the sorted subsets through the analysis of CD164 and CD34 expression. The scheme presented at the top left represents the path of differentiation from the most primitive fraction of CD34^{high}CD164^{high} cells. FIG. 22A comprises FACS plots showing the cell phenotype at day 0 (Starting population) and in differentiation states at day 4 in expansion culture, or at day 14 in Mk and My culture conditions.

FIG. 22B comprises bar graphs summarizing the cytometric analysis described in FIG. 22A. Shown are Median±Error from 3 independent BM.

FIGS. 23A-23C depict human engraftment in PB and spleen of transplanted NBSGW mice. FIG. 23A comprises graphs depicting the composition of human CD45+ cells in murine PB at the indicated number of weeks post-transplant. Myeloid and lymphoid reconstitution were analyzed within the human CD45+ population. FIG. 23B is a bar graph showing human CD45+ cell engraftment in murine spleen at 16 weeks post-transplant. FIG. 23C is a bar graph showing the composition of human CD45+ cells showed in b). N=3-4 mice per group.

### DETAILED DESCRIPTION OF THE INVENTION

The invention features hematopoietic stem/progenitor cell compositions and methods of using such cells in transplantation.

The invention is based, at least in part, on the discovery that endolin (CD164) is a reliable marker for the earliest branches of HSPC specification in transplantation cell products. Hematopoietic Stem/Progenitor cells (HSPCs) are endowed with the role of maintaining a diverse pool of blood cells throughout the human life. Over the past few decades, studies of HSPCs in humans have focused on cells expressing the CD34 surface molecule, showing that they are heterogeneous and represent different stages of differentiation into multiple blood lineages. Single-cell RNA-Seq was used to stratify bone marrow CD34⁺ cells. This analysis revealed a hierarchically-structured transcriptional landscape of hematopoietic differentiation. Still, this landscape misses key early fate decisions. Provided herein is a broader profiling of lineage negative hematopoietic progenitors that recovers missing branchpoints into basophil/mast cells and monocytes, and reveals the complete underlying structure of adult hematopoiesis. This map has strong similarities in topology and gene expression to that found in mouse. Moreover, these analyses reveal that a population of CD34⁺CD164^{High} hematopoietic/stem progenitor cells are particularly useful for clinical transplantation and gene therapy.

### Methods of the Invention

The invention provides hematopoietic stem cells/progenitors expressing increased levels of CD164 (CD164^{high}), including cells that are CD34⁺CD164^{High}. A hematopoietic stem cell, isolated from bone marrow, blood, cord blood, fetal liver and yolk sac, is the progenitor cell that generates blood cells or following transplantation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) When transplanted into lethally irradiated animals or humans, hematopoietic stem cells can repopulate the erythroid, neutrophil-macrophage, megakaryocyte, and lymphoid hematopoietic cell pool. In vitro, hematopoietic stem cells can be induced to undergo at least some self-renewing cell divisions and can be induced to differentiate to the same lineages observed in vivo.

It is well known in the art that hematopoietic cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

Hematopoietic stem cells can be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated blood products can be enriched for cells having “hematopoietic stem cell” characteristics in a number of ways. For example, the blood product can be depleted from the more differentiated progeny. The more mature, differentiated cells can be selected against, via cell surface molecules they express. Additionally, the blood product can be fractionated by selecting for CD164⁺ alone or in combination with CD34⁺ cells. CD164⁺ cells (e.g., CD164^{high} cells) provide a subpopulation of cells capable of identifying the most primitive progenitor cells. Cells expressing increased amounts of CD164 (e.g., CD164^{high} cells) can be distinguished from those cells expressing lower amounts of this surface antigen (CD164^{low} cells). Such selection can be accomplished using, for example, magnetic beads having an anti-CD164 antibody fixed to their surface (Dynal, Lake Success, N.Y.). Unfractionated blood products can be obtained directly from a donor or retrieved from cryopreservative storage.

Biological samples may comprise mixed populations of cells, which can be purified to a degree sufficient to produce a desired effect. Those skilled in the art can readily determine the percentage of hematopoietic stem cells or their progenitors in a population using various well-known methods, such as fluorescence activated cell sorting (FACS). Purity of the hematopoietic stem cells can be determined according to the genetic marker profile within a population. Dosages can be readily adjusted by those skilled in the art (e.g., a decrease in purity may require an increase in dosage). In several embodiments, it will be desirable to first purify the cells. Hematopoietic stem cells of the invention preferably comprise a population of cells that have about 50-55%, 55-60%, 60-65% and 65-70% purity (e.g., cells not expressing the desired marker (e.g., CD164) have been removed or are otherwise absent from the population). More preferably the purity is about 70-75%, 75-80%, 80-85%; and most preferably the purity is about 85-90%, 90-95%, and 95-100%.

### Treatment Methods Related to CD164⁺ Hematopoietic Stem/Progenitor Cells

In one aspect, the methods of the invention can be used to treat any disease or disorder in which it is desirable to increase the amount of CD164^{high} hematopoietic stem/progenitor cells and support the maintenance or survival of such cells. Preferably, for transplantation purposes, the hematopoietic stem/progenitor cells are primitive hematopoietic stem cells and early myeloid progenitors (e.g., CD34⁺CD164^{high} cells).

Frequently, subjects in need of the inventive treatment methods disclosed herein will be those undergoing or expecting to undergo a hematopoietic cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy must be terminated to allow the hematopoietic system to replenish the blood cell supplies before a patient is re-treated with chemotherapy.

Thus, methods of the invention can be used, for example, to treat patients requiring a bone marrow transplant or a hematopoietic stem cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkin's lymphoma, or leukemia.

Disorders treated by methods of the invention can be the result of an undesired side effect or complication of another primary treatment, such as radiation therapy, chemotherapy, or treatment with a bone marrow suppressive drug, such as zidovudine, chloramphenicol, or ganciclovir. Such disorders include neutropenias, anemias, thrombocytopenia, and immune dysfunction. In addition, methods of the invention can be used to treat damage to the bone marrow caused by unintentional exposure to toxic agents or radiation.

Methods of the invention can further be used as a means to increase the amount of mature cells derived from hematopoietic stem cells (e.g., erythrocytes). For example, disorders or diseases characterized by a lack of blood cells, or a defect in blood cells, can be treated by increasing the pool of hematopoietic stem cells. Such conditions include thrombocytopenia (platelet deficiency), and anemias such as aplastic anemia, sickle cell anemia, Fanconi's anemia, and acute lymphocytic anemia. In addition to the above, further conditions which can benefit from treatment using methods of the invention include, but are not limited to, lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV induced ITP, myelodysplasia; thrombocytotic disease, thrombocytosis, congenital neutropenias (such as Kostmann's syndrome and Schwachman-Diamond syndrome), neoplastic associated neutropenias, childhood and adult cyclic neutropenia; post-infective neutropenia; myelodysplastic syndrome; and neutropenia associated with chemotherapy and radiotherapy.

The disorder to be treated can also be the result of an infection (e.g., viral infection, bacterial infection or fungal infection) causing damage to hematopoietic stem cells.

Immunodeficiencies, such as T and/or B lymphocytes deficiencies, or other immune disorders, such as rheumatoid arthritis and lupus, can also be treated according to the methods of the invention. Such immunodeficiencies may also be the result of an infection (for example infection with HIV leading to AIDS), or exposure to radiation, chemotherapy, or toxins. Also benefiting from treatment according to methods of the invention are individuals who are healthy, but who are at risk of being affected by any of the diseases or disorders described herein (“at-risk” individuals). At-risk individuals include, but are not limited to, individuals who have a greater likelihood than the general population of becoming cytopenic or immune deficient. Individuals who were previously treated for cancer, e.g., by chemotherapy or radiotherapy, and who are being monitored for recurrence of the cancer for which they were previously treated; and individuals who have undergone bone marrow transplantation or any other organ transplantation, or patients anticipated to undergo chemotherapy or radiation therapy or be a donor of stem cells for transplantation are candidates for treatment with the methods of the invention. In some treatment protocols, B cell progenitor cells are specifically excluded from the therapeutic cells administered to one in need.

A reduced level of immune function compared to a normal subject can result from treatment with specific pharmacological agents, including, but not limited to chemotherapeutic agents to treat cancer; certain immunotherapeutic agents; radiation therapy; immunosuppressive agents used in conjunction with bone marrow transplantation; and immunosuppressive agents used in conjunction with organ transplantation.

### Genetically Altered Hematopoietic Stem Cells

In some embodiments, subjects are treated with a CD34⁺CD164^{high} hematopoietic stem/progenitor cell, or progeny thereof, that is genetically altered. Genetic alteration of a hematopoietic progenitor cell includes all transient and stable changes of the cellular genetic material, which are created by the addition of exogenous genetic material. Examples of genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Natural genetic changes such as the spontaneous rearrangement of a T cell receptor gene without the introduction of any agents are not included in this embodiment. Exogenous genetic material includes nucleic acids or oligonucleotides, either natural or synthetic, that are introduced into the hematopoietic progenitor cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with diethylaminoethanol (DEAE), transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half-life, and the like. Polymeric delivery systems also have been used successfully to deliver nucleic acids into cells, as is known by those skilled in the art. Such systems even permit oral delivery of nucleic acids.

In the present invention, the preferred method of introducing exogenous genetic material into hematopoietic cells is by transducing the cells in situ on the matrix using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of hematopoietic cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target hematopoietic cell may or may not result in stable transduction. Certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into hematopoietic cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the hematopoietic cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., 1991, Proc. Natl. Acad. Sci. USA, 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA, 86:10006-10010), and other constitutive promoters known to those of skill in the art. In addition, many viral promoters function constitutively in eukaryotic cells. These include: the early and late promoters of simian virus 40 (SV40); the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic adenosine monophosphate (cAMP) promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified hematopoietic cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding a therapeutic agent, the expression vector preferably includes a selection gene, for example, a neomycin resistance gene, for facilitating selection of hematopoietic cells that have been transfected or transduced with the expression vector. Alternatively, the hematopoietic cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The selection and optimization of a particular expression vector for expressing a specific gene product in an isolated hematopoietic cell is accomplished by obtaining the gene, preferably with one or more appropriate control regions (e.g., promoter, insertion sequence); preparing a vector construct comprising the vector into which is inserted the gene; transfecting or transducing cultured hematopoietic cells in vitro with the vector construct; and determining whether the gene product is present in the cultured cells.

A variety of genes can be delivered according to the methods of the invention, to correct or lessen the impact of a genetic defect or deficiency in blood cells (e.g., a variant or non-functioning adenosine deaminase (ADA) gene or Wiskott-Aldrich Syndrome protein (WASP)). Additionally, hematopoietic stem cells or their progeny can be used as a vehicle to deliver a transgenic product to non-hematopoietic tissues. For example, these genetically modified cells, or their progeny, can deliver interferon alpha (IFNa) to tumors or, because a hematopoietic stem cell (or its progeny) can cross the blood-brain barrier, it can deliver a therapeutic compound such as arylsulfatase A (ARSA) to the brain.

### Culture of Hematopoietic Stem/Progenitor Cells

Employing the culture conditions described in greater detail below, it is possible according to the invention to preserve hematopoietic progenitor cells and to stimulate the expansion of hematopoietic progenitor cell number and/or colony forming unit potential. Once expanded, the cells, for example, can be returned to the body to supplement, replenish, etc. a patient's hematopoietic progenitor cell population. This might be appropriate, for example, after an individual has undergone chemotherapy.

It also is possible to stimulate cells of the invention with hematopoietic growth agents that promote hematopoietic cell maintenance, expansion and/or differentiation, and also influence cell localization, to yield the more mature blood cells, in vitro. Such expanded populations of blood cells may be applied in vivo as described above, or may be used experimentally as will be recognized by those of ordinary skill in the art. Such differentiated cells include those described above, as well as T cells, plasma cells, erythrocytes, megakaryocytes, basophils, polymorphonuclear leukocytes, monocytes, macrophages, eosinophils and platelets.

In some embodiments, it may be desirable to maintain the selected cells in culture for hours, days, or even weeks prior to administering them to a subject. Media and reagents for tissue culture are well known in the art (see, for example, Pollard, J. W. and Walker, J. M. (1997) Basic Cell Culture Protocols, Second Edition, Humana Press, Totowa, N.J.; Freshney, R. I. (2000) Culture of Animal Cells, Fourth Edition, Wiley-Liss, Hoboken, N.J.). Examples of suitable media for incubating/transporting CD34⁺CD164^{high} hematopoietic stem/progenitor cell samples include, but are not limited to, Dulbecco's Modified Eagle Medium (DMEM), Roswell Park Memorial Institute (RPMI) media, Hanks' Balanced Salt Solution (HBSS) phosphate buffered saline (PBS), and L-15 medium. Examples of appropriate media for culturing cells of the invention include, but are not limited to, DMEM, DMEM-F12, RPMI media, EpiLlfe medium, and Medium 171. The media may be supplemented with fetal calf serum (FCS) or fetal bovine serum (FBS) as well as antibiotics, growth factors, amino acids, inhibitors or the like, which is well within the general knowledge of the skilled artisan.

The growth agents of particular interest for the culture of HSCs are hematopoietic growth factors. By hematopoietic growth factors, it is meant factors that influence the survival, proliferation or differentiation of hematopoietic progenitor cells. Growth agents that affect only survival and proliferation, but are not believed to promote differentiation, include the interleukins 3, 6 and 11, stem cell factor and FLT-3 ligand. Hematopoietic growth factors that promote differentiation include the colony stimulating factors such as GMCSF, GCSF, MCSF, Tpo, Epo, Oncostatin M, and interleukins other than IL-3, 6 and 11. The foregoing factors are well known to those of ordinary skill in the art. Most are commercially available. They can be obtained by purification, by recombinant methodologies or can be derived or synthesized synthetically.

When cells are cultured without any of the foregoing agents, it is meant that the cells are cultured without the addition of such agent except as may be present in serum ordinary nutritive media or within the blood product isolate, unfractionated or fractionated, which contains the hematopoietic progenitor cells.

### Cell Transplantation

Current practice during bone marrow transplantation involves the isolation of bone marrow cells from the bone marrow and/or peripheral blood of donor subjects. Human hematopoietic progenitor cells and human subjects are particularly important embodiments. One of skill in the art would be aware of methods for isolating hematopoietic stem cells from peripheral blood. For example, blood in PBS is loaded into a tube of Ficoll (Ficoll-Paque, Amersham) and centrifuged at 1500 rpm for 25-30 minutes. After centrifugation the white center ring is collected as containing hematopoietic stem cells.

Hematopoietic progenitor cell manipulation is also useful as a supplemental treatment to chemotherapy, e.g., hematopoietic progenitor cells may be caused to localize into the peripheral blood and then isolated from a subject that will undergo chemotherapy, and after the therapy the cells can be returned (e.g. ex vivo therapy may also be performed on the isolated cells). Thus, the subject in some embodiments is a subject undergoing or expecting to undergo an immune cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy must be terminated to allow the hematopoietic system to replenish the blood cell supplies before a patient is retreated with chemotherapy. Cells of the invention may be provided to such patients, where they will engraft, and provide a stable population of hematopoietic stem/progenitor cells capable of restoring the patients' hematopoietic system.

Once hematopoietic progenitor cells are mobilized from the bone marrow to the peripheral blood a blood sample can be isolated in order to obtain the hematopoietic progenitor cells. These cells can be transplanted immediately or they can be processed in vitro first. For instance, the cells can be expanded in vitro and/or they can be subjected to an isolation or enrichment procedure. It will be apparent to those of ordinary skill in the art that the crude or unfractionated blood products can be enriched for cells having increased levels of CD164 (e.g., CD34⁺CD164^{high}), which identify cells having “hematopoietic progenitor cell” characteristics. Some of the ways to enrich include, e.g., depleting the blood product from the more differentiated progeny. The more mature, differentiated cells can be selected against, via cell surface molecules they express. Additionally, the blood product can be fractionated selecting for CD34⁺CD164^{high} hematopoietic stem/progenitor cells. Such selection can be accomplished using, for example, magnetic anti-CD164 beads.

### Formulations

Compositions of the invention comprising purified CD34⁺CD164^{high} hematopoietic stem/progenitor cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON′S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the genetically modified immunoresponsive cells as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of CD34⁺CD164^{high} hematopoietic stem/progenitor cells, including genetically modified cells, is the quantity of cells necessary to achieve an optimal effect. The quantity of cells to be administered will vary for the subject being treated. In a one embodiment, between 10⁴ to 10⁸, between 10⁵ to 10⁷, or between 10⁶ and 10⁷ genetically modified immunoresponsive cells of the invention are administered to a human subject. In preferred embodiments, at least about 1×10⁷, 2×10⁷, 3×10⁷, 4×10⁷, and 5×10⁷ genetically modified immunoresponsive cells of the invention are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the invention. Typically, any additives (in addition to the active stem cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Administration of Cells

Compositions comprising a selected cell of the invention (e.g., CD34⁺CD164^{high} hematopoietic stem/progenitor cell) can be provided systemically or directly to a subject for the treatment or prevention of a disease or disorder characterized by a deficiency in such cells. The cells can be administered in any physiologically acceptable vehicle, normally intravascularly, although they may also be introduced into other convenient site where the cells may find an appropriate site for regeneration and differentiation. In one approach, at least 100,000, 250,000, or 500,000 cells is injected. In other embodiments, 750,000, or 1,000,000 cells is injected. In other embodiments, at least about 1×10⁵ cells will be administered, 1×10⁶, 1×10⁷, or even as many as 1×10⁸ to 1×10¹⁰, or more are administered. Selected cells of the invention can comprise a purified population of cells (e.g., CD34⁺CD164^{high} that expresses CD164 and other markers of hematopoietic stem cells known in the art. Those skilled in the art can readily determine the percentage of cells in a population using various well-known methods, such as fluorescence activated cell sorting (FACS). Preferable ranges of purity in populations comprising selected cells are about 50 to about 55%, about 55 to about 60%, and about 65 to about 70%. More preferably the purity is at least about 70%, 75%, or 80% pure, more preferably at least about 85%, 90%, or 95% pure. In some embodiments, the population is at least about 95% to about 100% selected cells. Dosages can be readily adjusted by those skilled in the art (e.g., a decrease in purity may require an increase in dosage). The cells can be introduced by injection, catheter, or the like.

Compositions of the invention include pharmaceutical compositions comprising genetically modified cells or their progenitors and a pharmaceutically acceptable carrier. Administration can be autologous or heterologous. For example, immunoresponsive cells, or progenitors can be obtained from one subject, and administered to the same subject or a different, compatible subject.

Selected cells of the invention or their progeny (e.g., in vivo, ex vivo or in vitro derived) can be administered via localized injection, including catheter administration, systemic injection, localized injection, intravenous injection, or parenteral administration. When administering a therapeutic composition of the present invention (e.g., a pharmaceutical composition containing a selected cell), it will generally be formulated in a unit dosage injectable form (solution, suspension, emulsion).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### CD164⁺ Hematopoietic Stem/Progenitor Cells

In humans, there have been conflicting proposals for the hierarchical relationships linking different hematopoietic progenitors. In the conventional depiction of human hematopoiesis, supported by lineage-tracing studies in the mouse, the earliest branching split lymphoid versus myelo/erythroid fate commitment. Conversely, in a recent challenge of the classical view, it has been suggested that multipotent progenitors could undergo a very early fate decision towards the megakaryocyte lineage followed by a single step-wise transition to either erythroid, myeloid and lymphoid commitment. The advent of single-cell RNA sequencing (scRNA-Seq) has created an opportunity to clarify the nature of human hematopoiesis through the study of transcriptional single-cell states, but also generated conflicting observations. Initial use of this technology in humans led to an alternative view that early hematopoiesis is composed by a continuum of low-primed undifferentiated hematopoietic stem and progenitor cells (CLOUD-HSPCs) from which unilineage-restricted cells emerge. Recently, scRNA-Seq data combined with assays of chromatin accessibility supported instead the notion of a structured hierarchy, revealing a variegated hematopoietic landscape, the existence of lineage-biased stem cells in mice, and of different stages of human lymphoid commitment in humans.

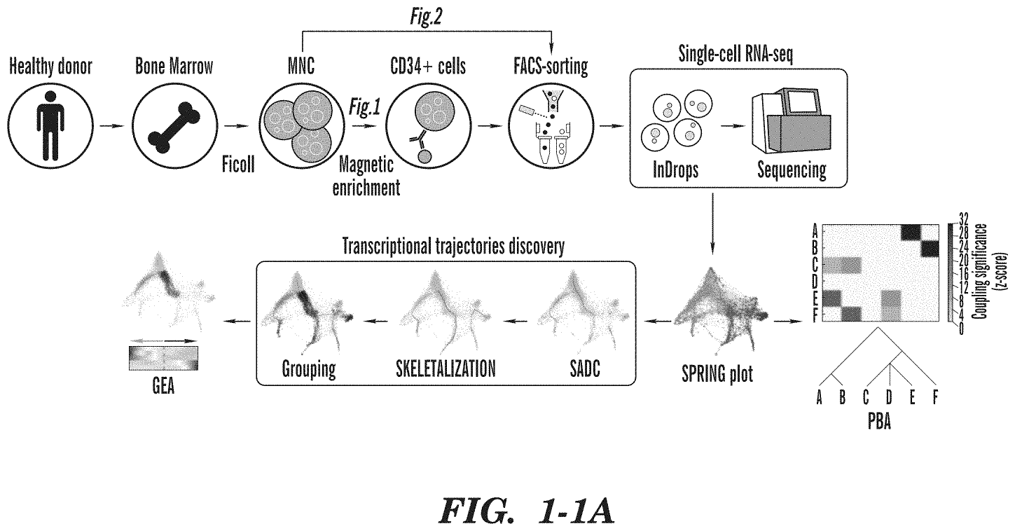

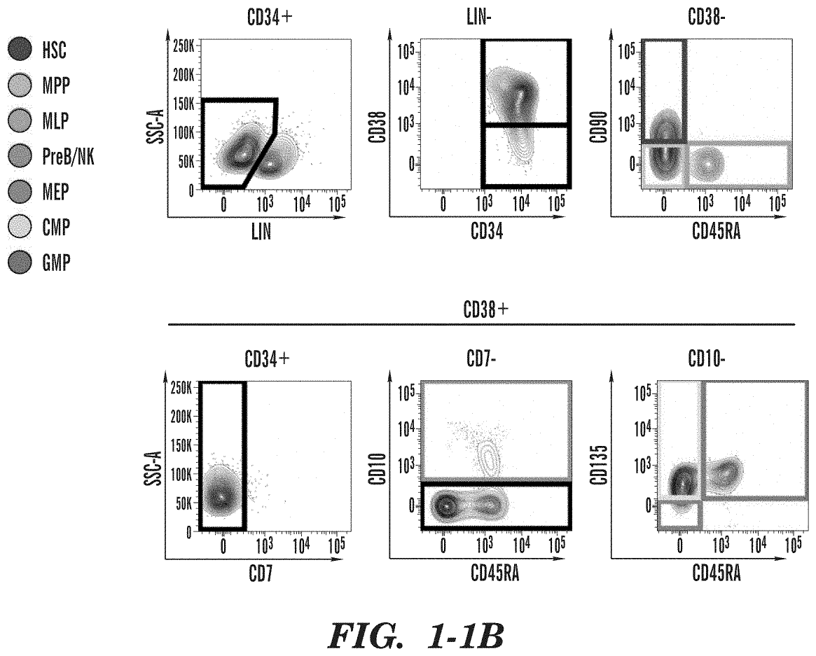

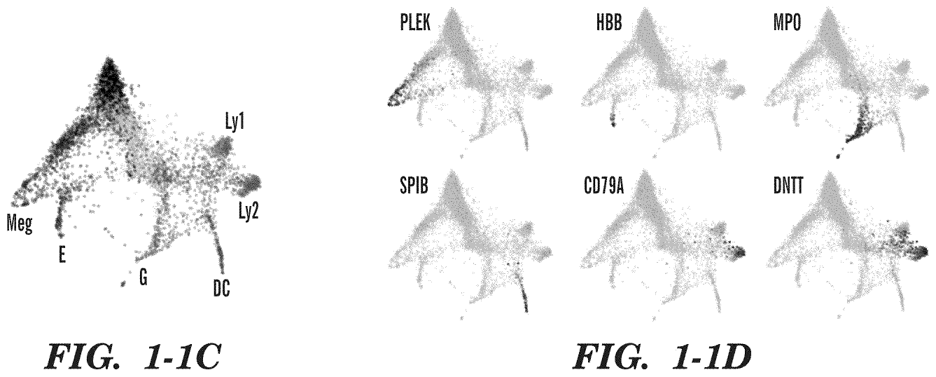

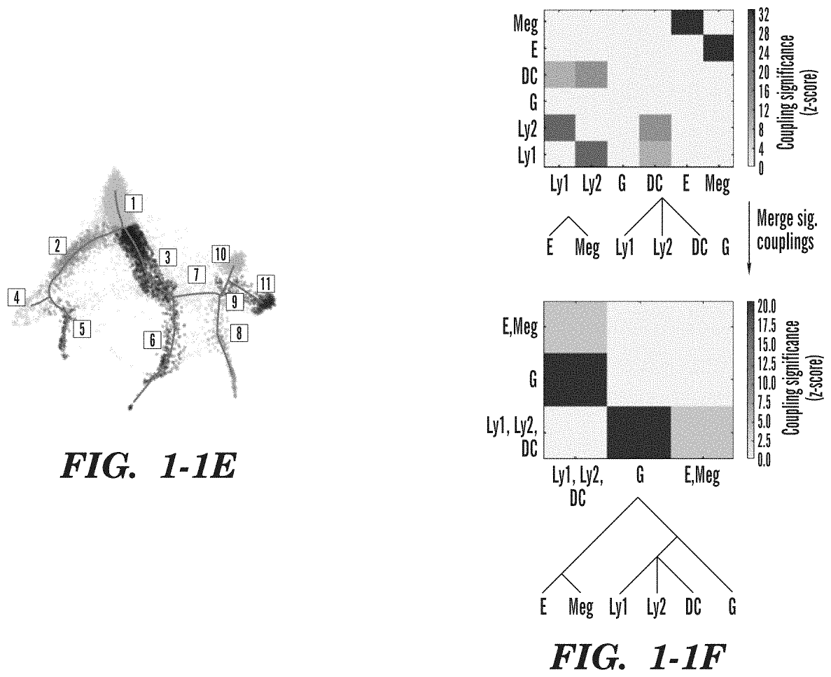

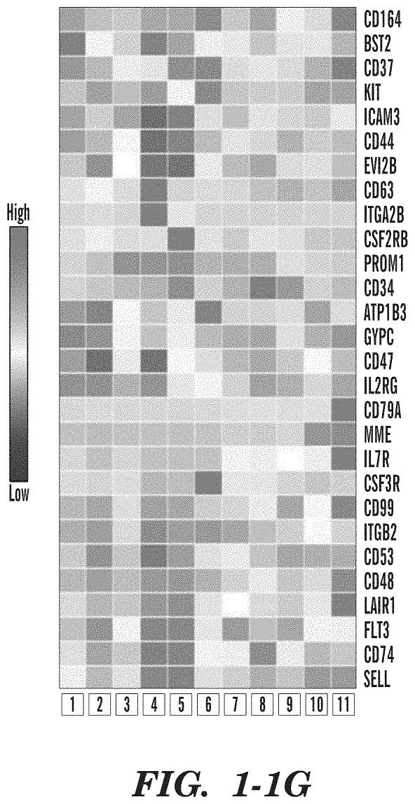

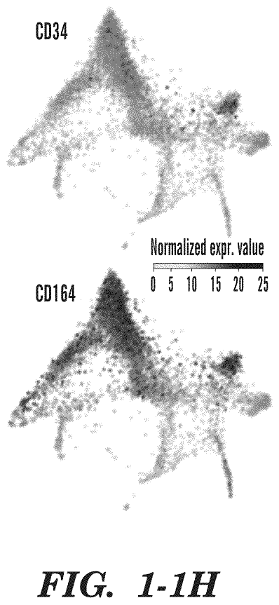

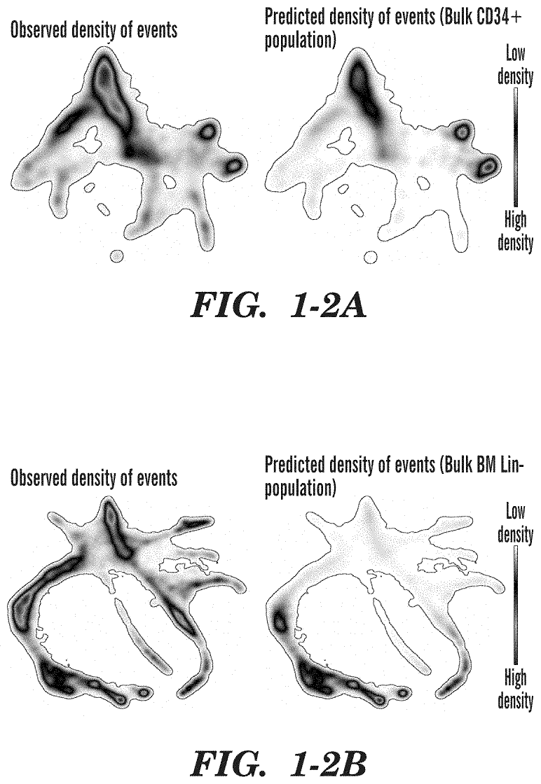

The population structure of early hematopoietic commitment was defined by profiling human HSPCs with high-throughput scRNA-Seq (FIG. 1-1A). In contrast to conventional methods, immature cells expressing the CD34 antigen, were not only isolated, but the analysis was extended to the whole bone marrow (BM) fraction lacking the main markers of terminal differentiation (Lineage negative, Lin− cells). This strategy differs from other attempts to profile early hematopoiesis in humans by deep scRNA-Seq, which have focused exclusively on the study of the whole CD34+ population (that comprise both Lin− and Lin+ cells), or on in silico modelling of the fate commitment of the CD34+ fraction containing the least differentiated HSPCs.

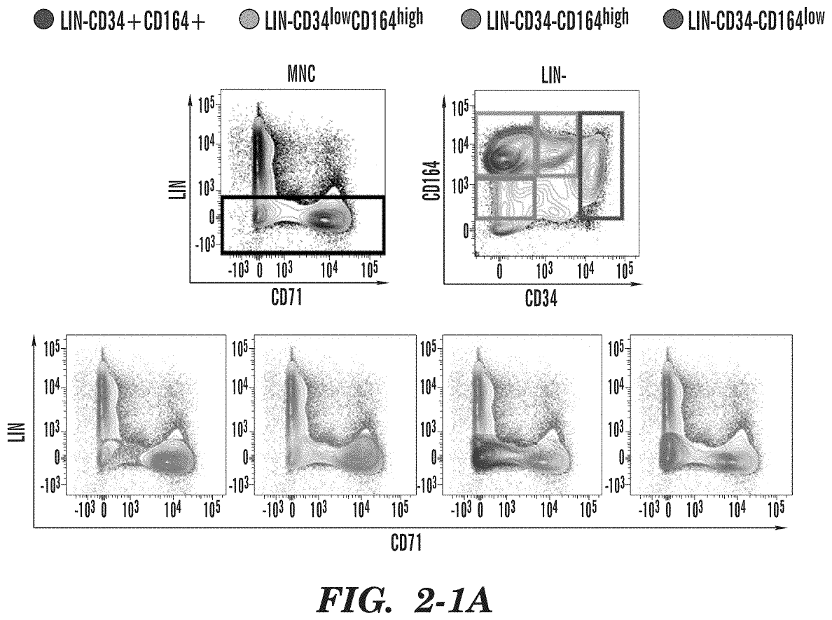

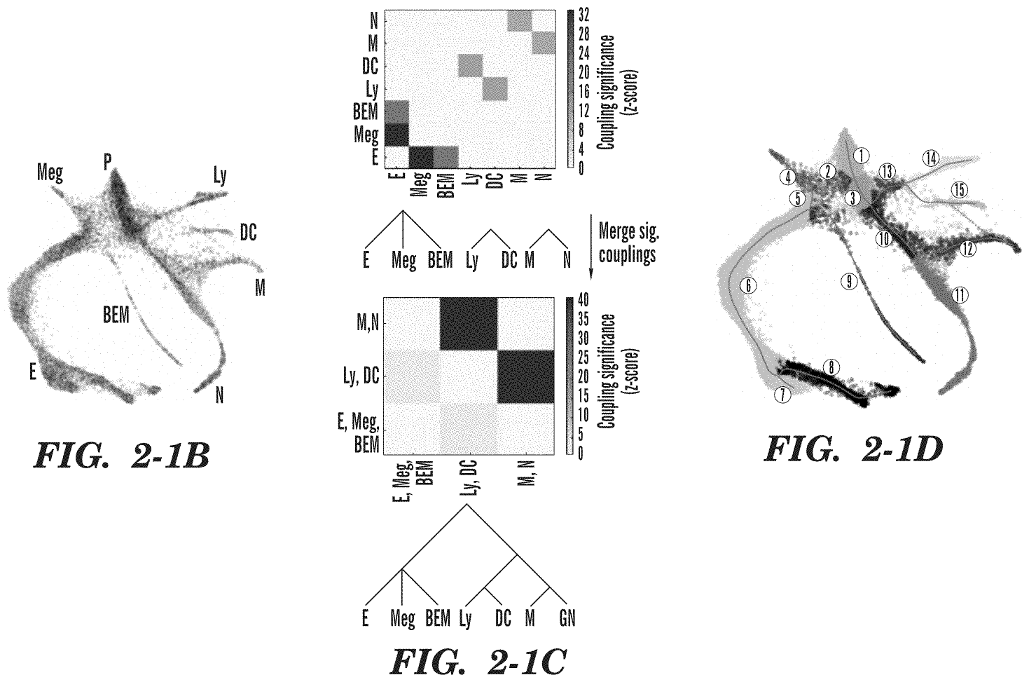

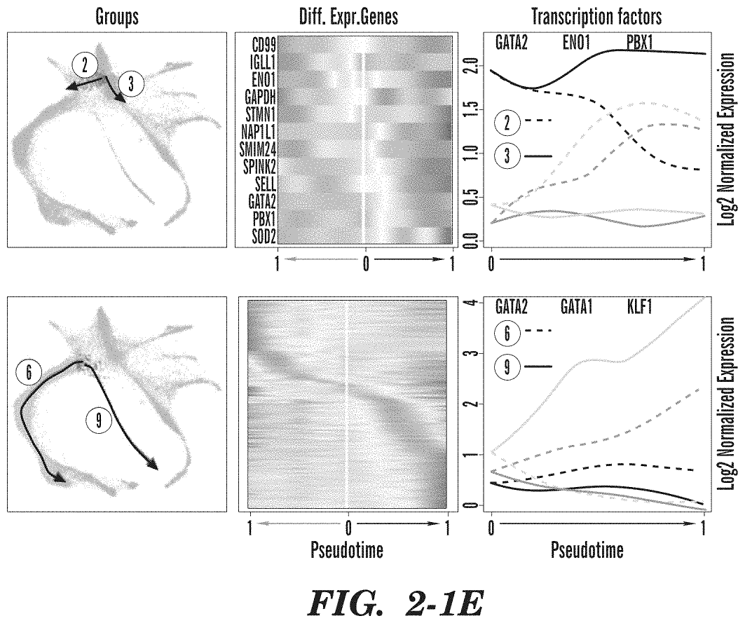

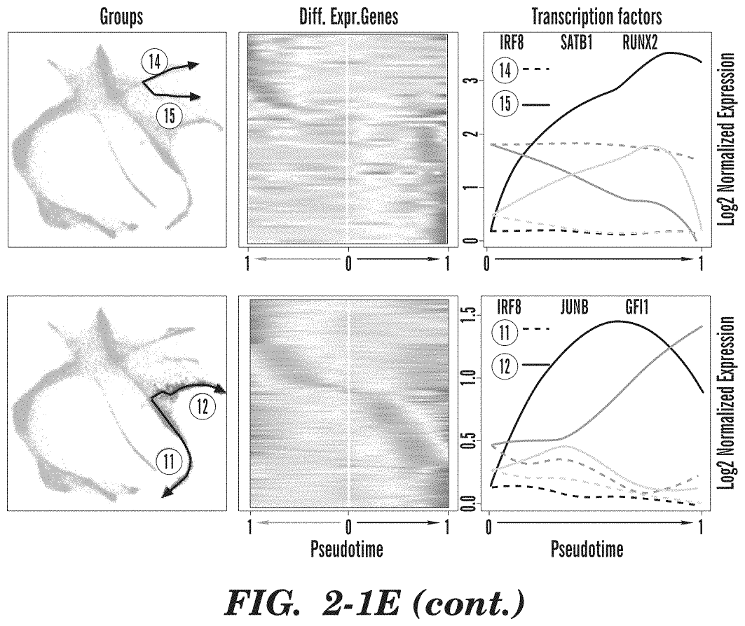

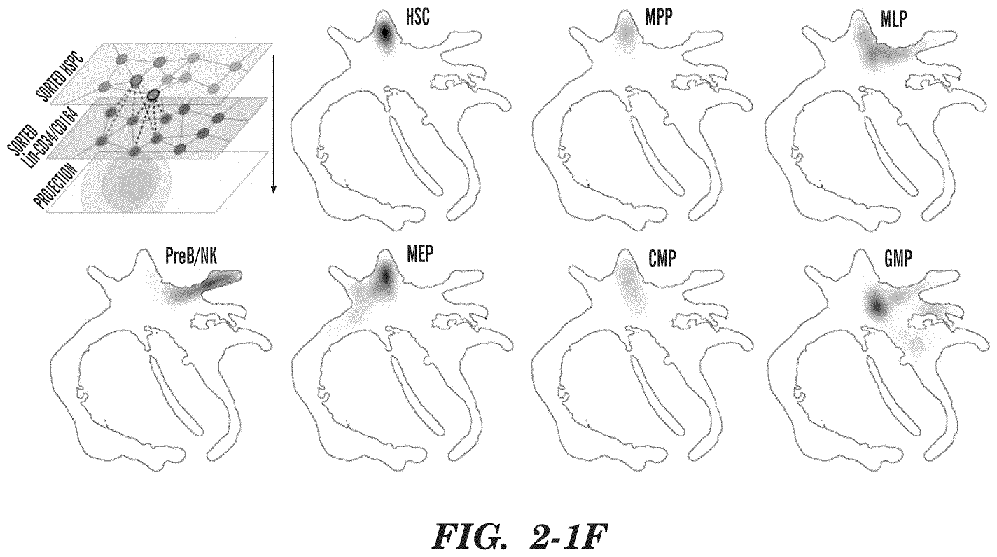

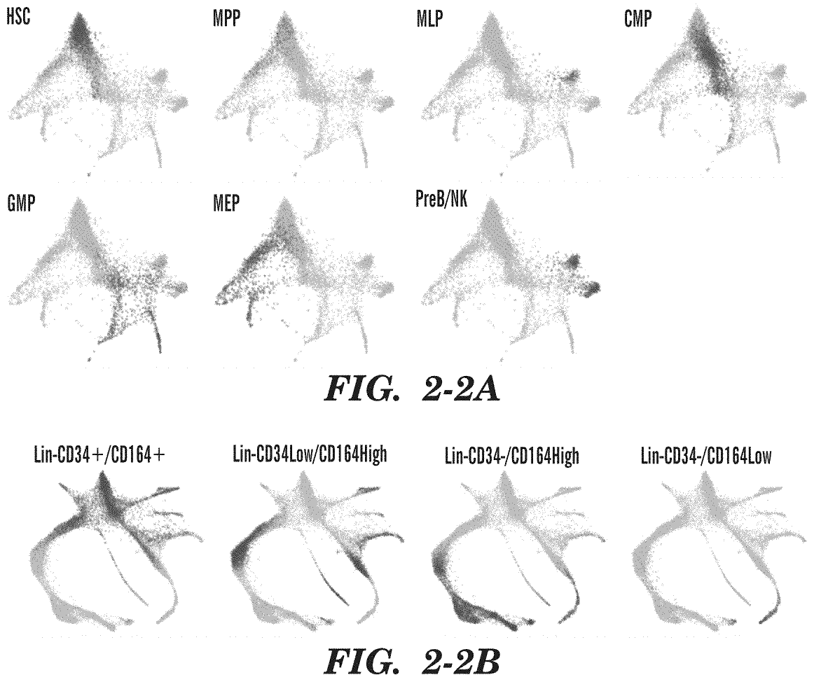

To establish a reference dataset and to address the heterogeneity and fate potential of the known CD34+ subsets, the first investigations were aimed at mapping at high resolution the single-cell transcriptional states of cells commonly defined as human HSPCs. To this goal, CD34+ cells purified by magnetic beads selection were separated into seven subpopulations, marking cells of differing fate potential (FIG. 1-1B) and tagged and sequenced the transcriptome of 6,011 single cells (FIG. 1-2A; Table 1A).

TABLE 1A

| | | Estimated | Post-filtering |
|---|---|---|---|
| Population | Sorted events | barcoded cells | barcoded cells |
| HSC | 25237 | 4000 | 1282 |
| MPP | 4133 | 1000 | 215 |
| MLP | 2186 | N.A* | 123 |
| PREB/NK | 2541 | N.A* | 592 |
| MEP | 21964 | 4000 | 1211 |
| CMP | 31832 | 4000 | 1576 |
| GMP | 16065 | 2000 | 1012 |

Gate on CD34+ cells
Lin−CD34+CD38−CD90+CD45RA−
Lin−CD34+CD38−CD90−CD45RA−
Lin−CD34+CD38−CD90−CD45RA+
Lin−CD34+CD38+CD7−CD10+
Lin−CD34+CD38+CD7−CD10−CD135−CD45RA−
Lin−CD34+CD38+CD7−CD10−CD135+CD45RA−
Lin−CD34+CD38+CD7−CD10−CD135+CD45RA+
*All the material was run with 90-95% cells receiving a barcode

The scRNA-Seq data was used to infer the structure of cell states in high-dimensional gene expression space (FIG. 1-1C and FIG. 1-2A). A visualization method (SPRING) previously developed for mouse hematopoietic progenitors was applied, whereby each cell represents a graph node, with graph edges linking nearest neighbor cells. The scRNA-Seq graph, visualized using a force-directed layout, shows a hierarchical, tree-like continuum of states, with branches that terminate at cells expressing recognizable transcriptional signatures of lineage commitment before the expression of final maturation markers (FIGS. 1-1C and 1-1D) [megakaryocytes (Meg), erythroid cells (E), granulocytes (G), dendritic cells (DC), lymphoid cells (Ly1-2)]. The structure of the single-cell data broadly partitions based on immunophenotypic sub-populations, but, significantly, the previously-defined HSPC subpopulations include substantial transcriptional heterogeneity (FIG. 2-2a).

The scRNA-Seq map of CD34⁺ sub-populations show that HSPCs do not undergo a single-step transition from CLOUD-HSPCs to unilineage states. Instead, they form a structured hierarchy (FIG. 1-1C). The earliest fate split separates erythroid-megakaryocyte progenitors from lymphoid-myeloid progenitors (LMPs), which separate further into lymphoid, DC and granulocytic progenitors. This hierarchy can be appreciated by inferring transcriptional trajectories using at least two independent algorithms that provide consistent results (FIGS. 1-1E, 1-1F and FIG. 3-2A). This indicates that human HSPCs are more organized than recently hypothesized, and show more structure than appreciated by classical immunophenotyping.

In the 1980s, the wide adoption of monoclonal antibodies for immunophenotyping revealed that the CD34 antigen is an effective marker to isolate immature HSPCs from humans. Since then, efforts have been made to define the hierarchical structure of HSPCs purified from immunomagnetic-selected CD34⁺ cells, under the assumption that this cell population effectively captures all early fate choices. While the above analysis supports such efforts, a focus on CD34⁺ cells purified with magnetic beads enrichment might provide an incomplete view of the earliest branching events in hematopoiesis. Branches towards basophils/eosinophils/mast cells and monocytes commitments were missing in the initial scRNA-Seq analysis of CD34 cells, despite these appearing as early events in mouse hematopoiesis. In addition, many cells negative for mature lineage markers in human BM are CD34^{low/-} and could account for additional transitional states at which CD34 expression is rapidly downregulated, thus greatly reducing their probability of capture. Therefore, to generate a complete landscape of early hematopoiesis, the analysis was extended to encompass human CD34^{low} and CD34⁻ cells. To this aim, four fractions of BM Lin− cells were collected from a second healthy donor, covering different degrees of maturation (FIG. 2-1A). The graded FACS-sorting used in this analysis corrects for expansion of cells as they differentiate, allowing examination of early states alongside later ones that comprise the vast majority of Lin− progenitors. In fractionating the cells by maturity, a cell surface marker, CD164, was used. CD164 was identified from the initial data set expressed by cells that are multipotent until just beyond the first E/Meg-LMP branch-point (FIGS. 1-1G and 1-1H). This fractionation strategy allowed preservation of the resolution of the single-cell events of the more primitive compartments, while at the same time maintaining a full representation of the late cell fate branching (FIG. 2-1B; FIG. 1-2B and FIG. 2-2B).

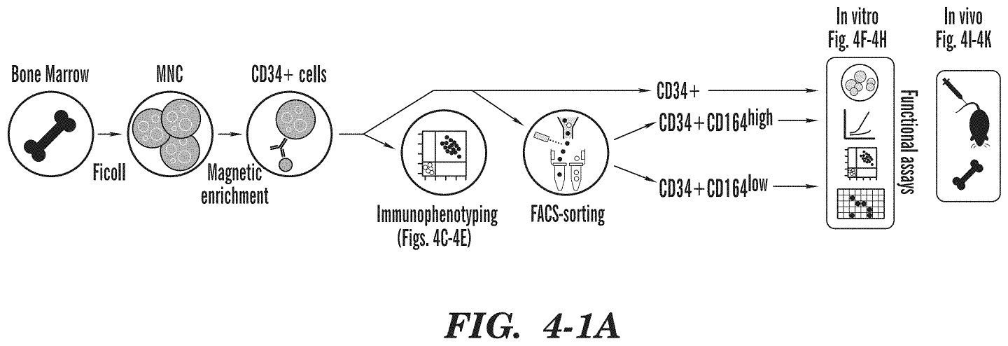

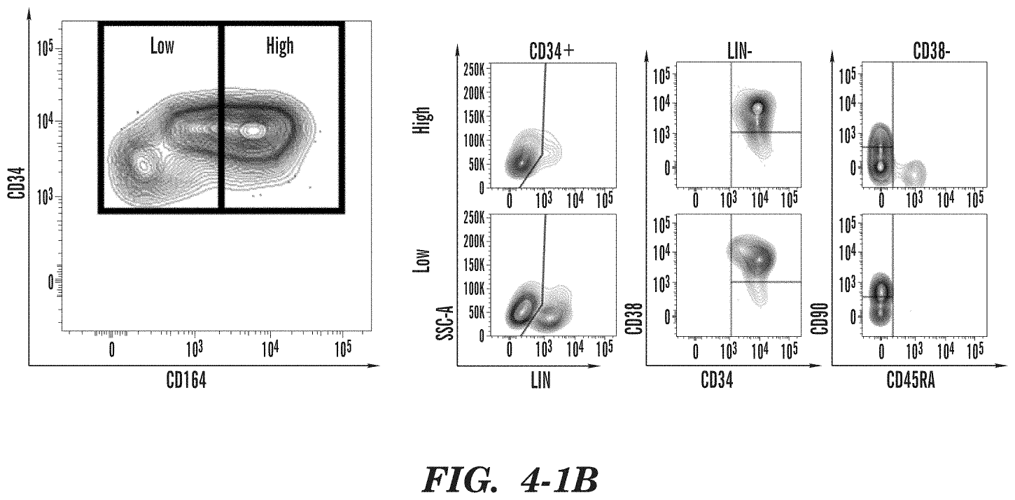

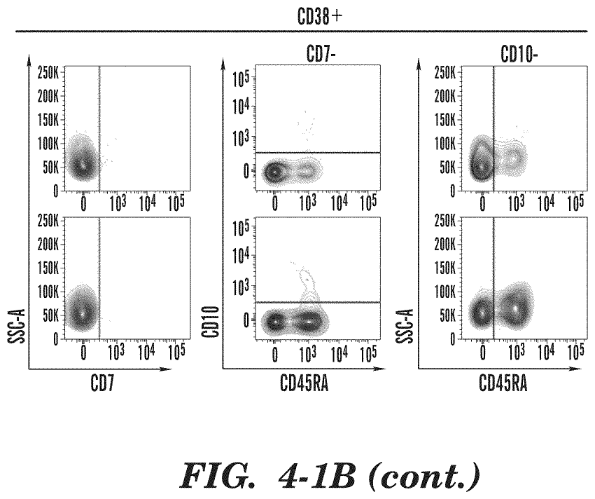

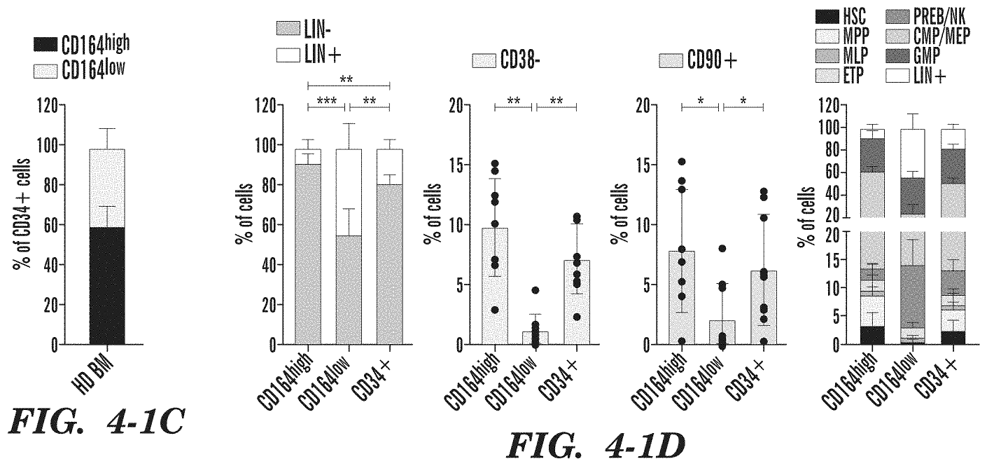

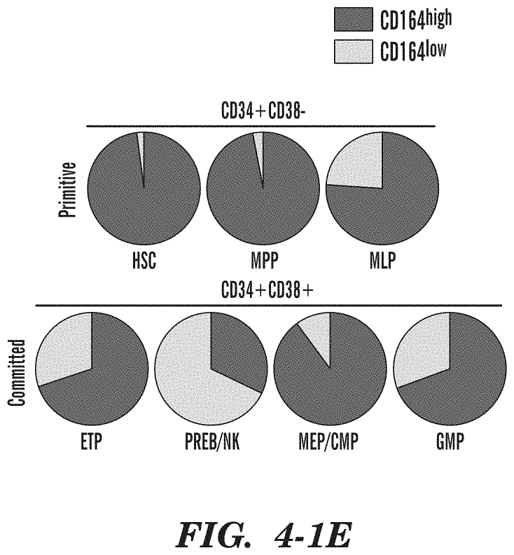

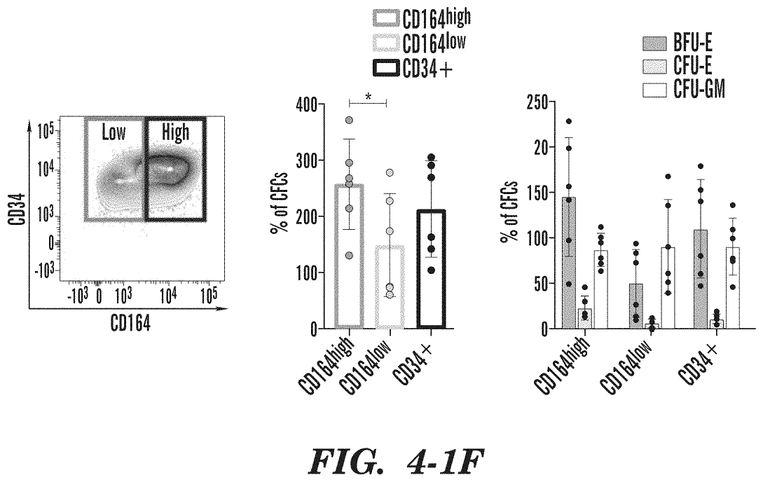

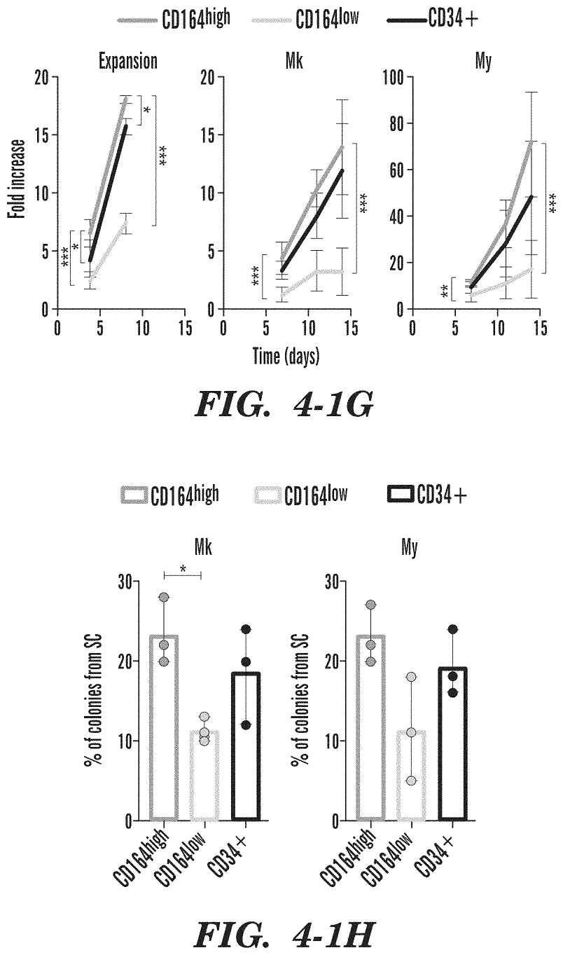

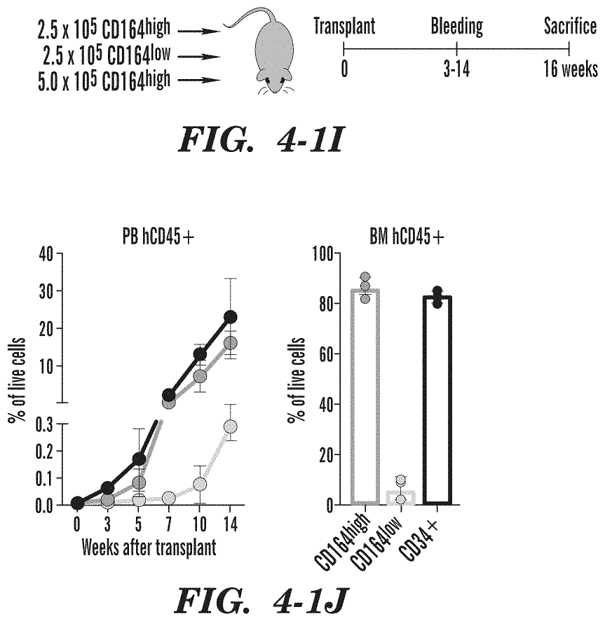

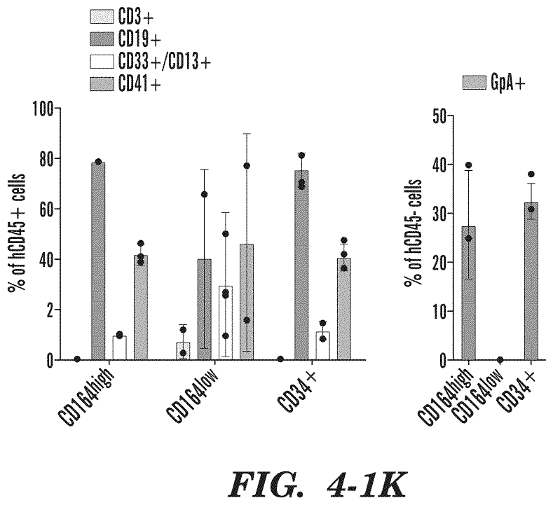

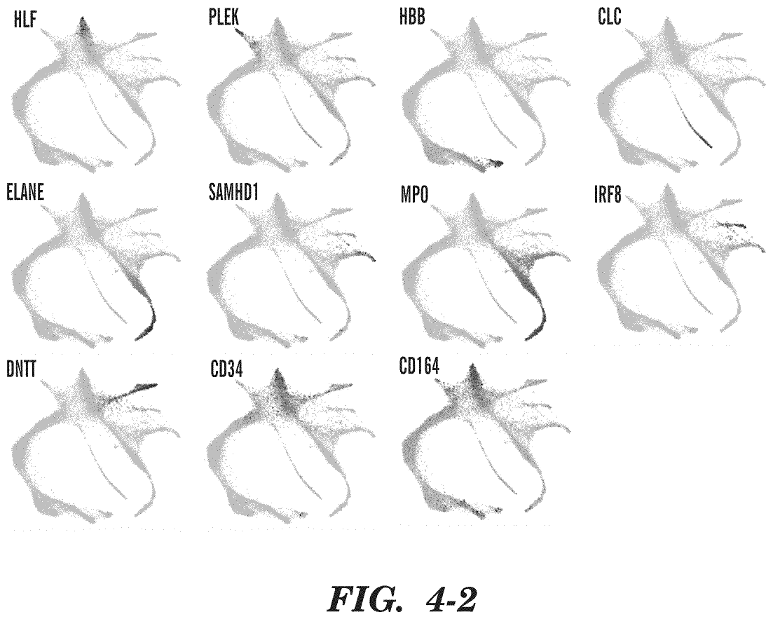

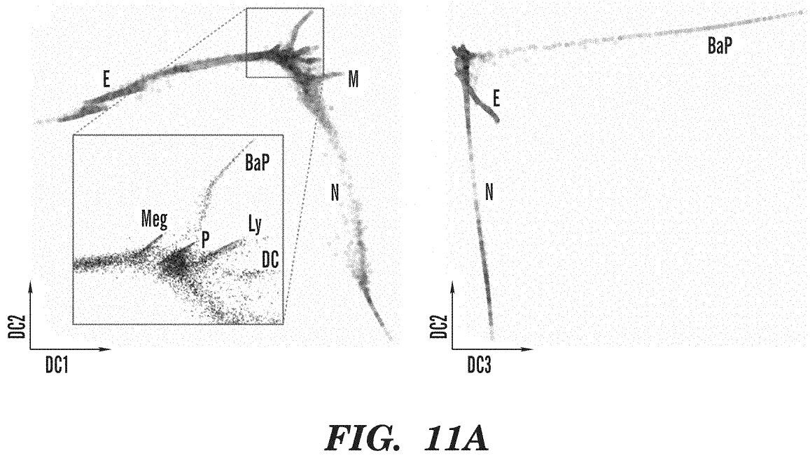

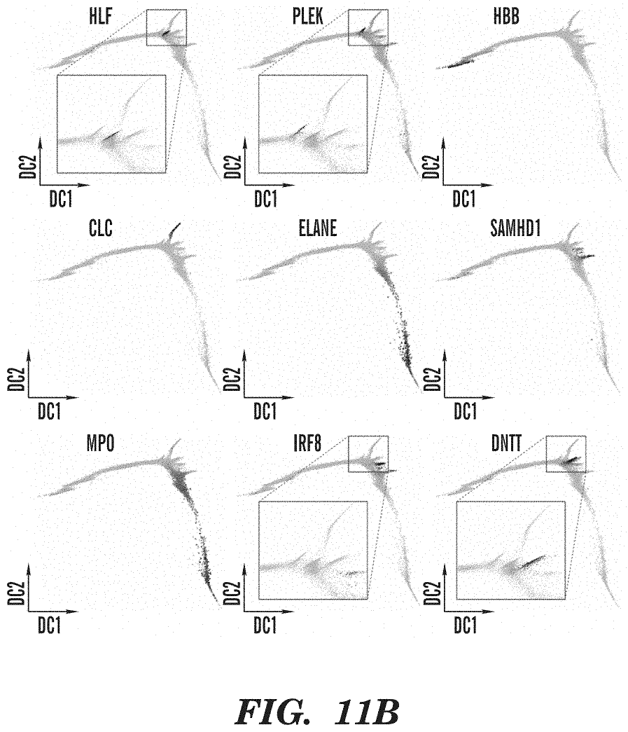

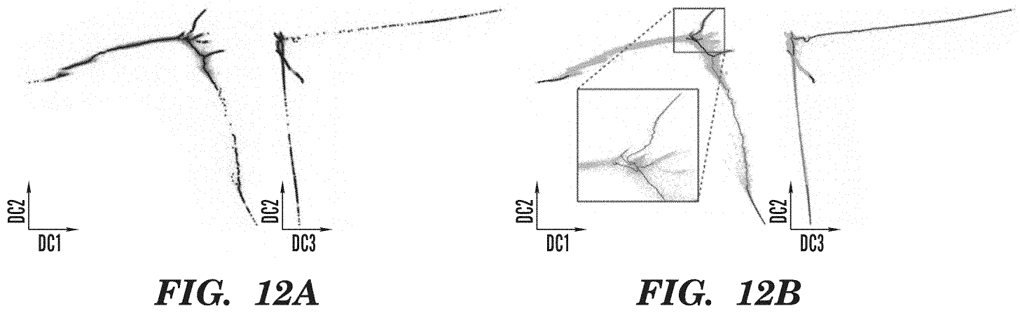

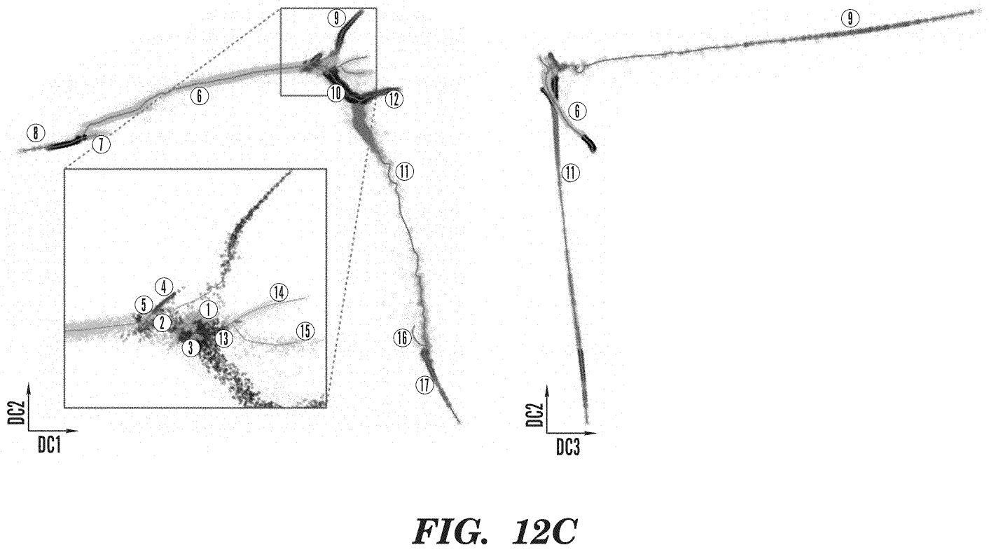

As predicted, the transcriptional map of the Lin− fraction, derived from the high-throughput clustering of 15,401 single-cells (FIG. 2-1B; Table 1B and FIG. 4-2), revealed important early features that were missing from the analysis of the immune-selected CD34⁺ population. Using the same graph-based technique as for CD34⁺ cells, the early basophils/eosinophils/mast cells (BEMs) and monocyte cell-fate decisions were identified. Notably, the newly identified class of BEM progenitors was found to associate with erythroid and megakaryocyte fates and not with granulocytes precursors. These data align with preliminary observations in human cord blood CD34⁺ cells. Monocyte progenitors, by contrast, emerge from a common neutrophil/monocyte precursor later in the myeloid commitment and after the branching decision towards DC progenitors, with a possible contribution from DC progenitors as recently shown in the mouse. This data also clarified the identity of the remaining CD34⁻ Lin⁻ cells, which consisted mostly of late neutrophil progenitors, and of a continuum of differentiating states towards erythroid commitment. As before, the same hierarchical structure could be computationally inferred using two independent approaches (PBA algorithm in FIG. 2-1C, and inferred transcriptional trajectories in FIGS. 2-1D and 3-2B and were confirmed upon analyzing the data with an independent method (FIGS. 11A, 11B, 12A, and 12B) that does not relay on a limited amount of k-nearest neighbors (kNN) for data-embedding calculation. To generate a resource for further study, the association between gene expression dynamics and cells progression along the estimated differentiation paths were investigated. Putative transcriptional switches occurring during early hematopoietic cell fate choice and genes exhibiting significant variations during lineage commitments were identified (FIGS. 2-1E and 5A-5D). This analysis contained valuable information for in vitro reprogramming efforts and for investigations into the origin of blood cell differentiation disorders and cancer.

TABLE 1B
Sorted events and estimated/post-filtering
barcoded cells in each population analyzed

| | | Estimated | Post-filtering |
|---|---|---|---|
| Population | Sorted events | barcoded cells | barcoded cells |
| Lin−CD34+CD164+ | 426759 | 5300 | 6343 |
| Lin−CD34lowCD164^{high} | 252909 | 5000 | 4266 |
| Lin−CD34−CD164^{high} | 663506 | 5000 | 4434 |
| Lin−CD34−CD164^{low} | 71220 | 300 | 358 |

Gate on MNC
Lin−CD34+CD164+
Lin−CD34lowCD164^{high}
Lin−CD34−CD164^{high}
Lin−CD34−CD164^{low}

To understand how the enrichment of CD34⁺ subsets could limit the view of early hematopoiesis, the CD34⁺ HSPCs sub-populations were projected on to the Lin− state map (FIG. 2-1F). The analysis confirmed that large portions of the Lin⁻ map are strongly under-represented upon the magnetic pulldown of the CD34⁺ population (namely the ones identifying BEMs, monocytes progenitors and the stages of late erythroid differentiation). This supports the concept that the Lin− population structure provides a more complete view of key cell fate decision along human hematopoietic commitment and suggests that, for a complete classification of HSPCs, analyses should be performed on FACS-sorted CD34+, CD34^{low} and CD34− compartments. Finally, this projection clarified the heterogeneous nature of the currently defined HSPC subsets, showing that they can be further fractionated into distinct and more homogenous transcriptional states (FIG. 6).

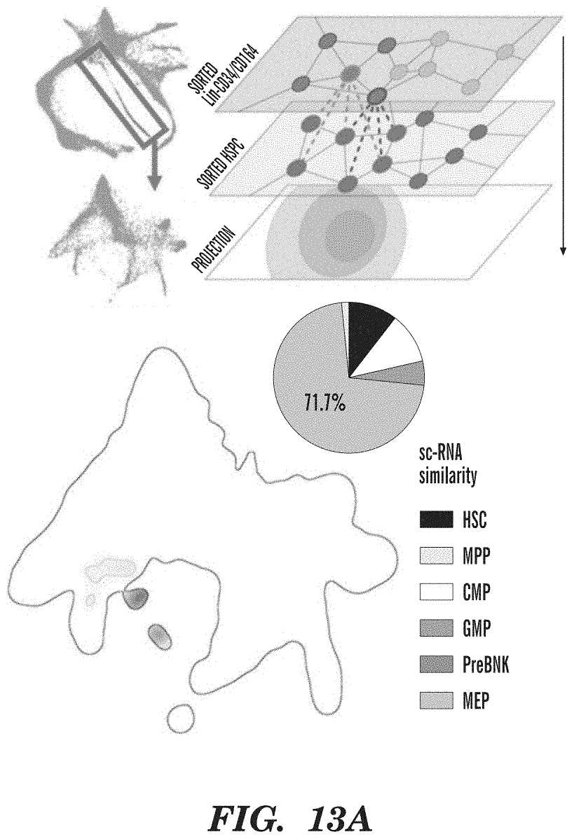

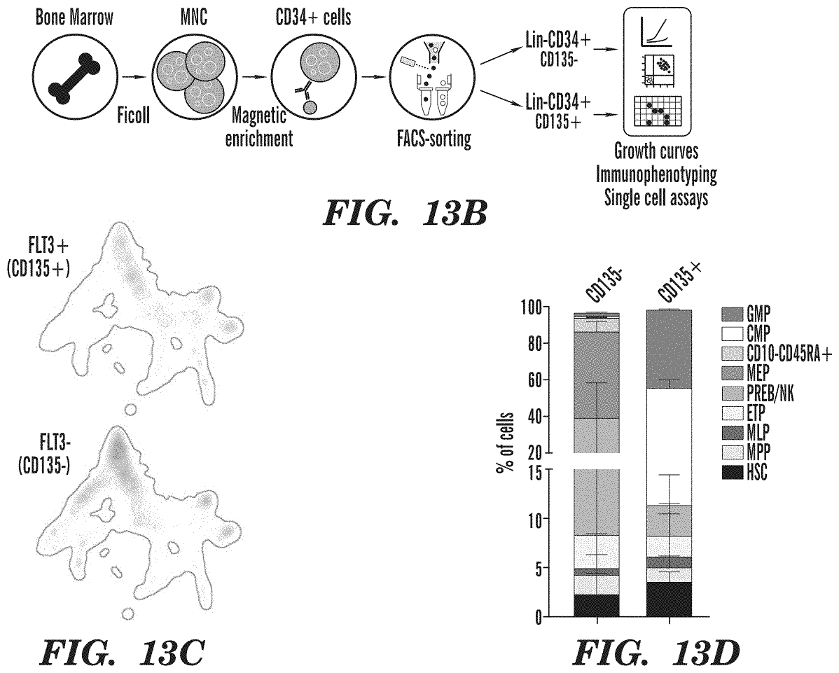

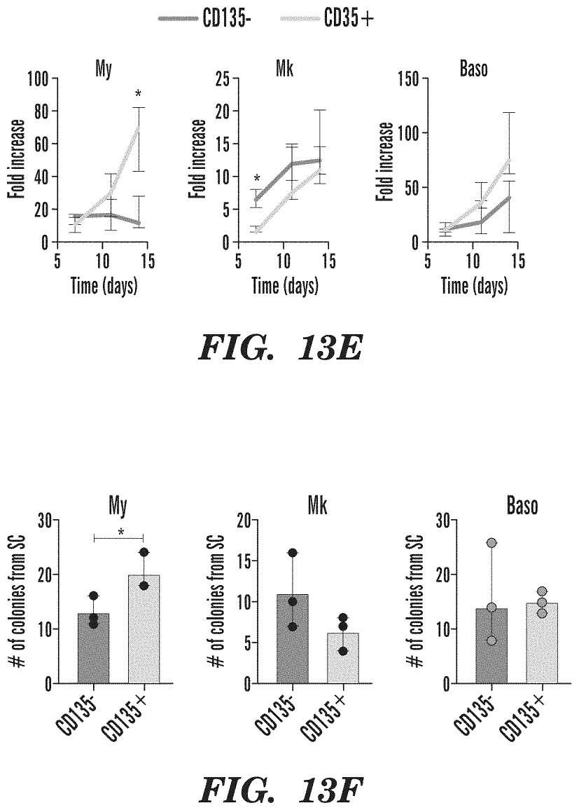

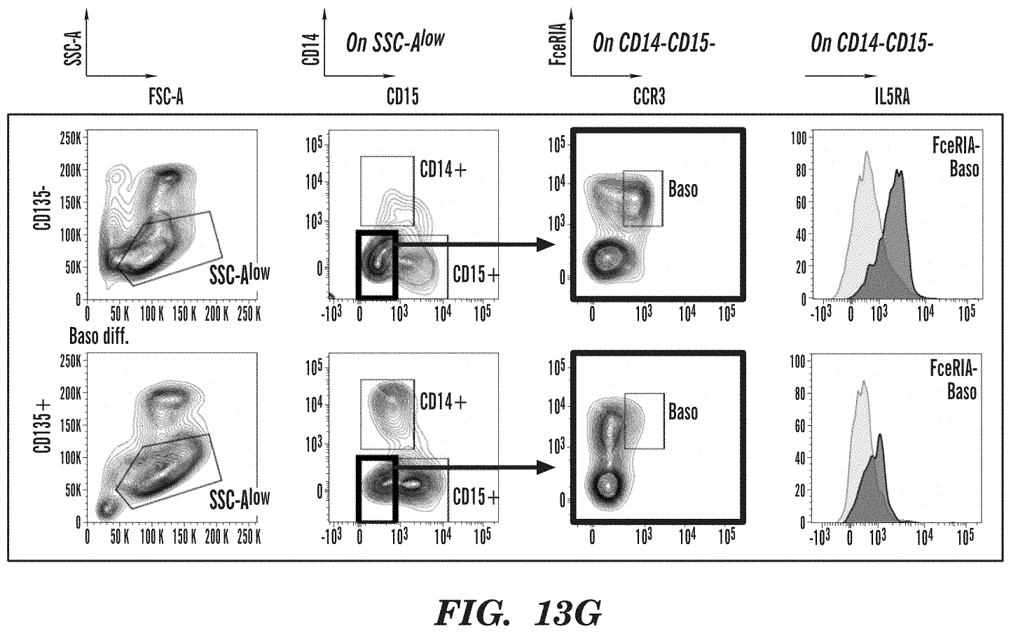

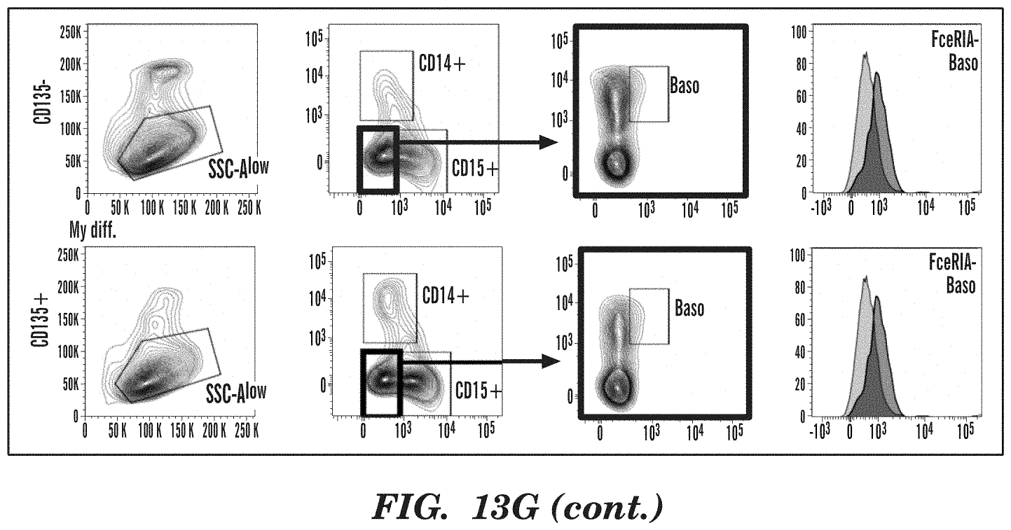

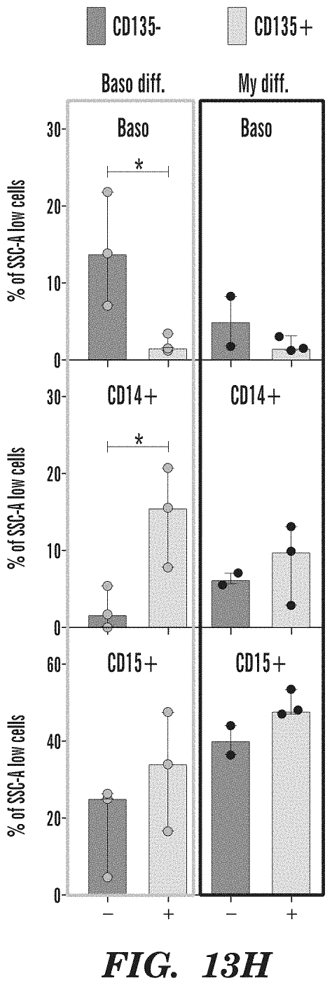

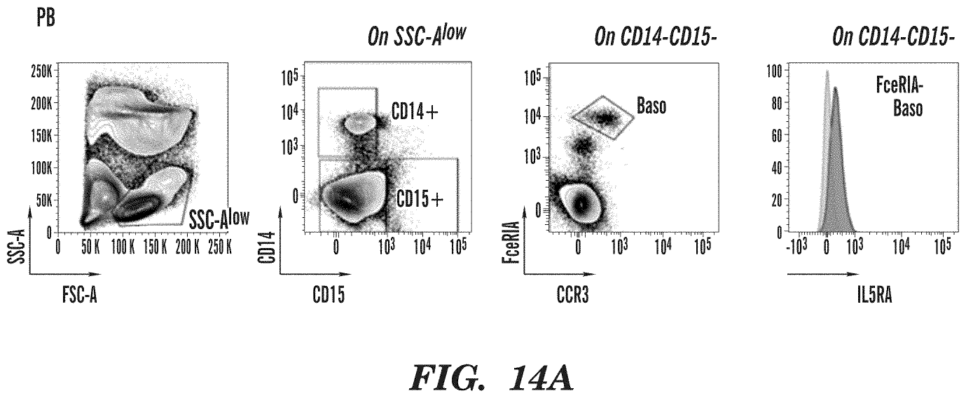

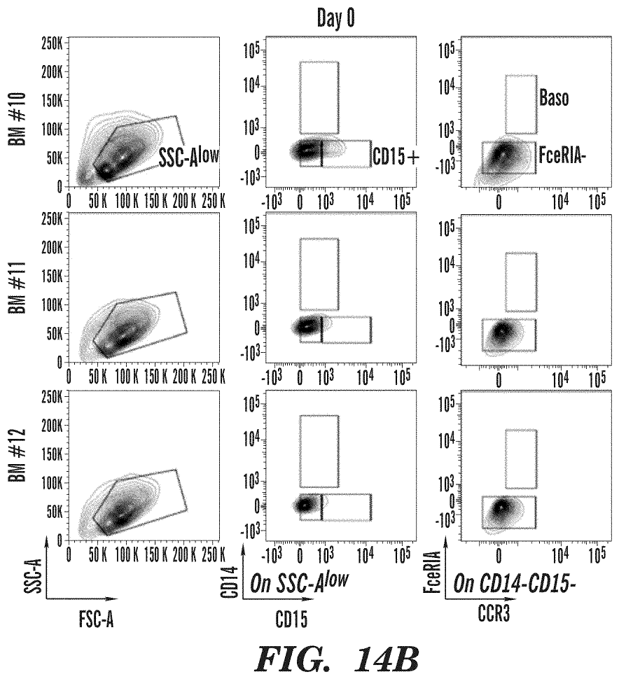

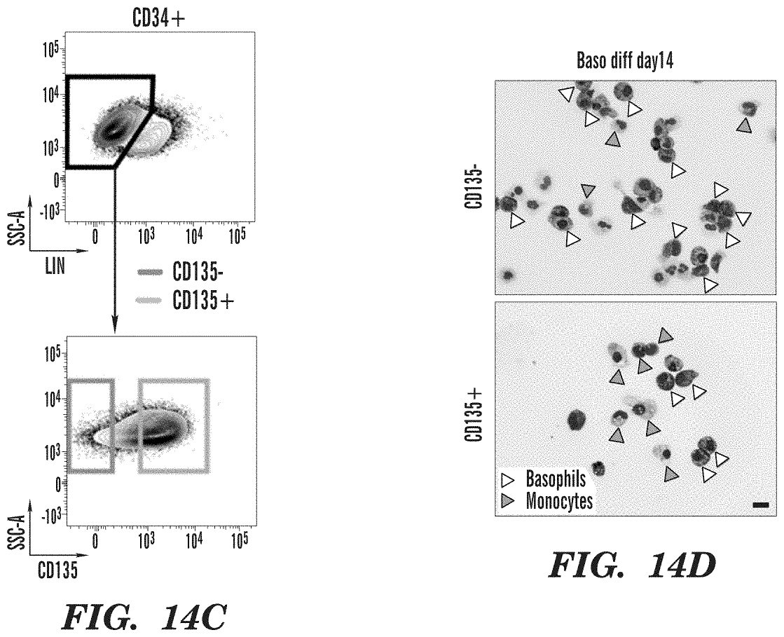

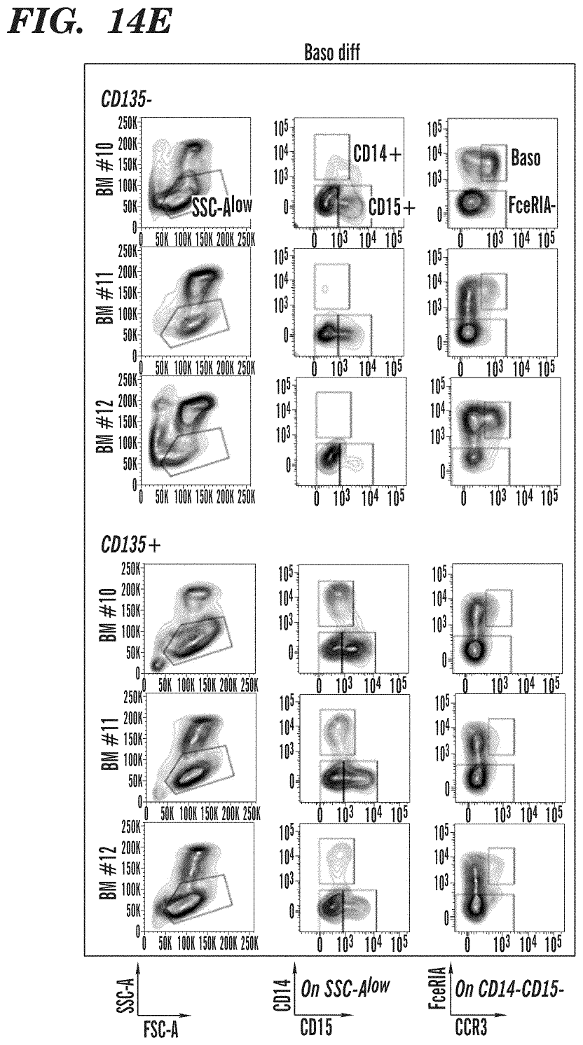

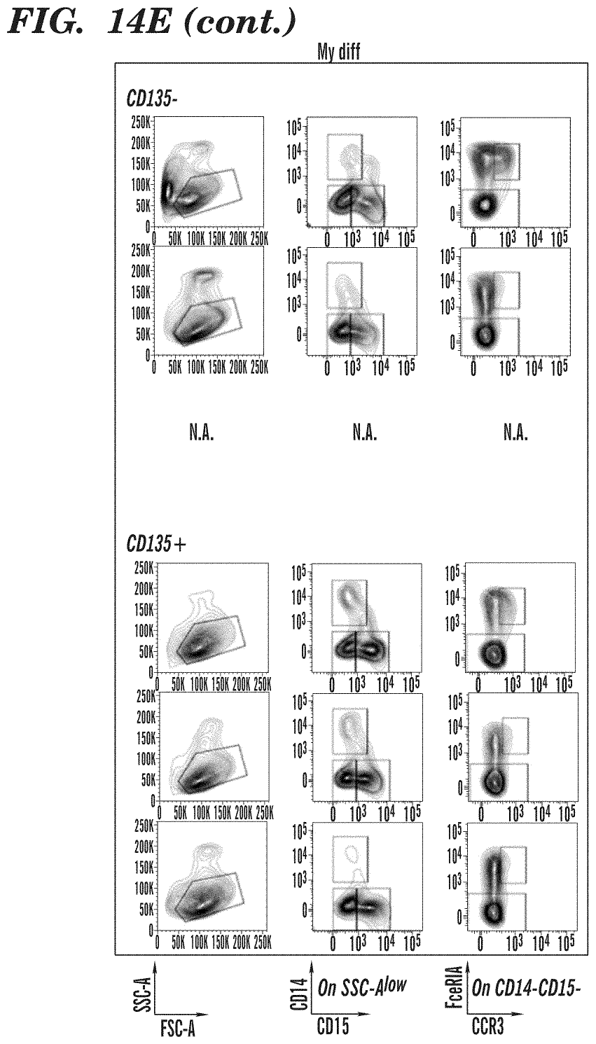

The most-notable result emerging from the exploration of the BM Lin− map was the identification of a branch toward cells carrying a transcriptional profile of early basophils specification. Strikingly, this class of basophils progenitors (BaP) was found to associate with erythroid and megakaryocyte fates and not with granulocytes precursors. The presently disclosed data, generated on adult human BM, align with and expand on preliminary observations in human cord blood CD34+ cells, and in murine hematopoiesis. To elaborate on this observation, the Basophil branch of the BM Lin− map was computationally projected onto the Lin− HSPC map to identify which, among the HSPC single cell states, had the highest scRNA similarity to this branch. The topological origin of the early basophil cell specification in the HSPC map was in striking accordance with what observed in the BM Lin− map and the highest level of similarity was detected with respect to the CD135− progenitors with known megakaryo-erythroid potential (MEP) (FIG. 13A). Building on these results, a series of in vitro differentiation assays were designed and conducted starting from FACS sorting Lin−CD34+ cells into CD135+ (FLT3+) (by definition containing common myeloid progenitors (CMP) and granulocyte-monocyte progenitors; GMP) and CD135−(FLT3−) (by definition containing MEP) cells (FIGS. 13B-13D and 14C). These two groups of cells were separately put in culture in myeloid-, megakaryocytes (MK)−, and basophil-differentiating conditions under the hypothesis that if basophils are generated by CMP or GMP (as suggested by the classical model of hematopoiesis) the CD135+ fraction should be the one capable of differentiating into basophils after culture. As reported in FIGS. 13A-13H, the Lin−CD34+4+CD135− and Lin−CD34+CD135+ populations had, as expected, specific growth preferences toward MK (the former) and myeloid (the latter) cell fates (FIGS. 13E and 13F). The two populations grew at similar rate in basophilic conditions, but while the Lin−CD34+CD135+ fraction generated mostly CD14+ monocytes (FIGS. 13G and 13H), the Lin−CD34+CD135− fraction emerged as the only population capable of giving rise with high efficiency to bona fide basophils (FIGS. 13G, 13H, 14B, 14D, and 14E) defined as SSC-AlowCD14-CD15−FceRIA+CCR3+IL5RA+ cells (as in Mori et al. 200930 and in immunophenotyping on human peripheral blood reported in FIG. 14A). This observation is in line with scRNA data showing that the basophil branch emerges from CD135− cells already committed toward a mixed MK/Erythroid/Basophil potential. Notably, because the experimental design purposely included also CD38− multipotent progenitors, one could have expected that basophils would have been generated at similar rates by the CD38− HSC/MPP that were present in both CD135+ and CD135− cell fractions (FIG. 13D). Conversely, the observation that only the CD135− cells were endowed with substantial basophilic potential strongly support the notion that the Lin−CD34+CD38−CD135− population might be already enriched in stem cells with very early priming towards a basophilic cell fate. The method of moment estimation of each HSPC subpopulation proportion with related standard deviation are provided in Table 2. The comparison of proportion estimates in the CD135− and CD135+ fractions have been calculated by means of the Student's t-test, under different variances hypothesis (BM #10,11,12).

TABLE 2
Statistical analysis on the HSPC bar graphs of FIG. 13D

| | | CD135− | | CD135+ | | | | |
|---|---|---|---|---|---|---|---|---|
| | | Parameter | Parameter | Parameter | Parameter | | | |
| | Parameter | Est. | Est. S.E. | Est. | Est. S.E. | T-test | | |
| Subpopulation | [P(Subpop)] | [MoM] | [MoM] | [MoM] | [MoM] | P-value | CD135− | CD135+ |
| HSC | pi:HSC | 2.420E−02 | 2.171E−02 | 3.077E−02 | 1.942E−02 | 7.16E−01 | — | — |
| MPP | pi:MPP | 5.827E−02 | 6.947E−02 | 3.728E−02 | 3.861E−02 | 6.77E−01 | — | — |
| MLP | pi:MLP | 1.091E−02 | 7.681E−03 | 9.381E−03 | 3.809E−03 | 7.78E−01 | — | — |
| ETP | pi:ETP | 3.408E−02 | 2.237E−03 | 2.629E−02 | 1.481E−02 | 4.59E−01 | — | — |
| PREB/NK | pi:PREB/NK | 3.289E−01 | 1.700E−01 | 4.136E−02 | 1.742E−02 | 9.80E−02 | — | — |
| MEP | pi:MEP | 4.480E−01 | 1.103E−01 | NA | NA | NA | ▴ | ▾ |
| CD10−CD45+ | pi:CD10−CD45+ | 7.306E−02 | 3.282E−02 | NA | NA | NA | ▴ | ▾ |
| CMP | pi:CMP | 1.989E−02 | 8.857E−03 | 4.570E−01 | 3.234E−02 | 9.49E−04 | ▾ | ▴ |
| GMP | pi:GMP | 2.694E−03 | 2.332E−03 | 3.979E−01 | 6.380E−02 | 8.51E−03 | ▾ | ▴ |

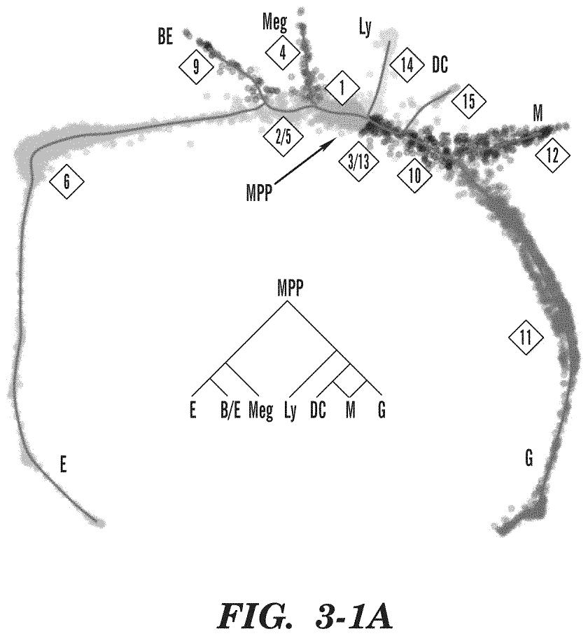

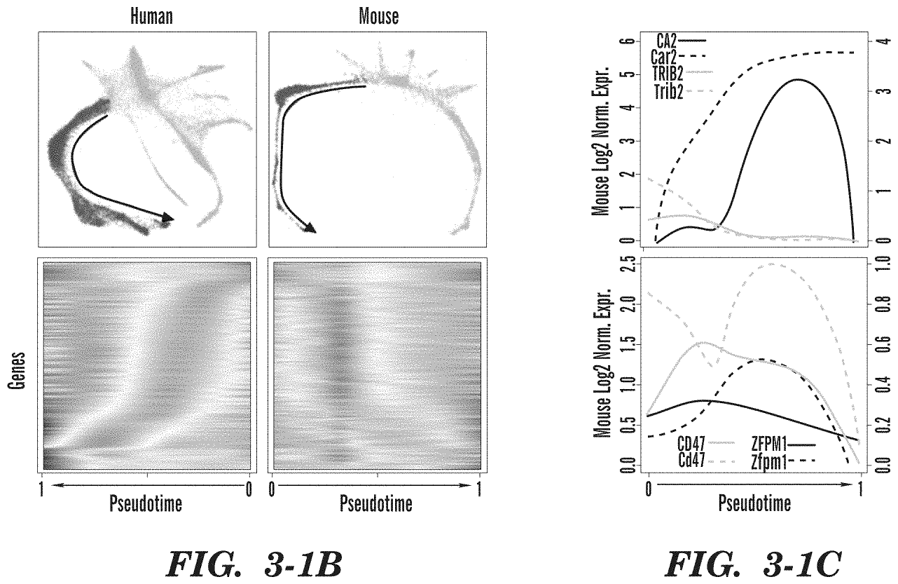

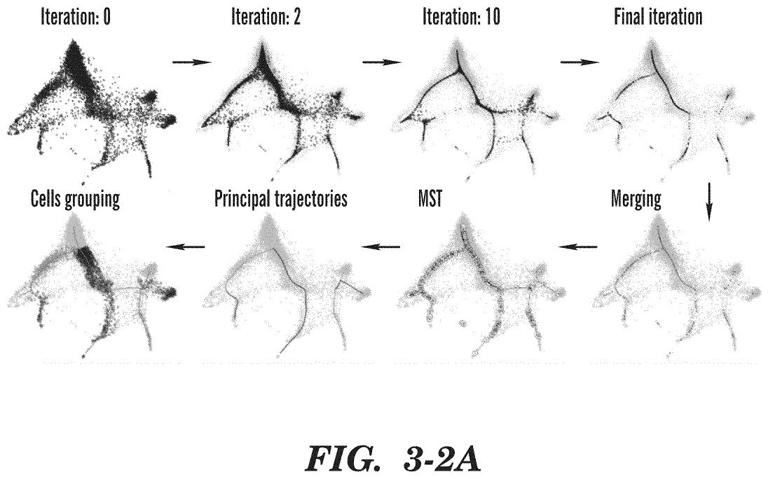

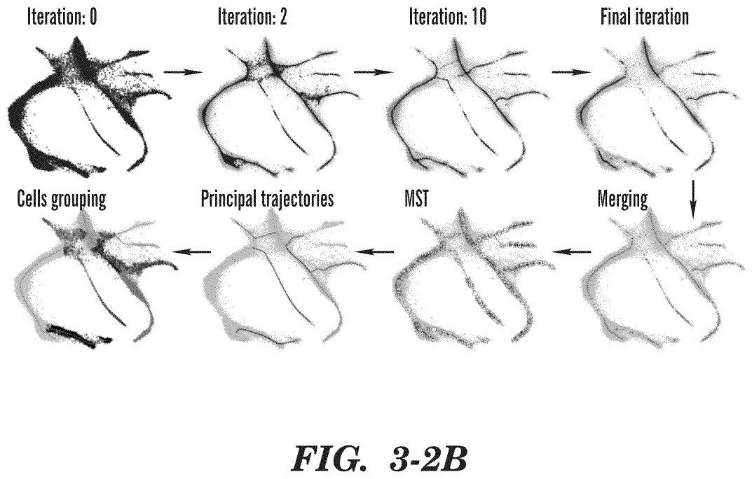

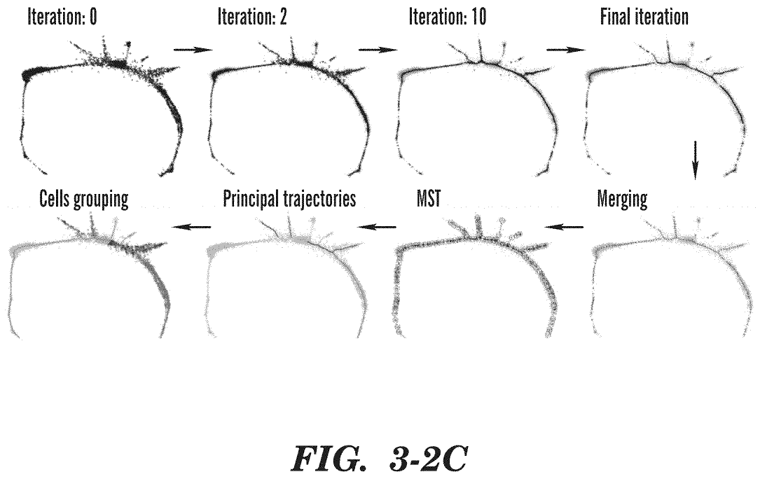

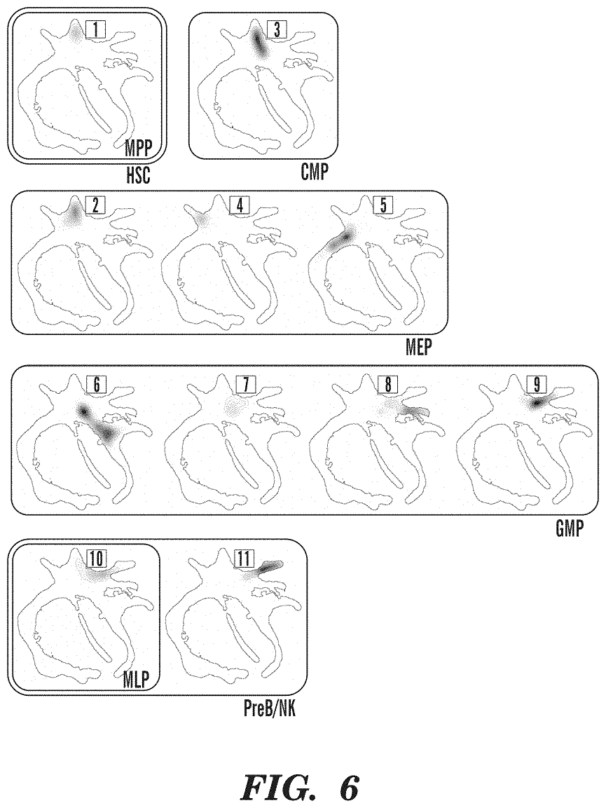

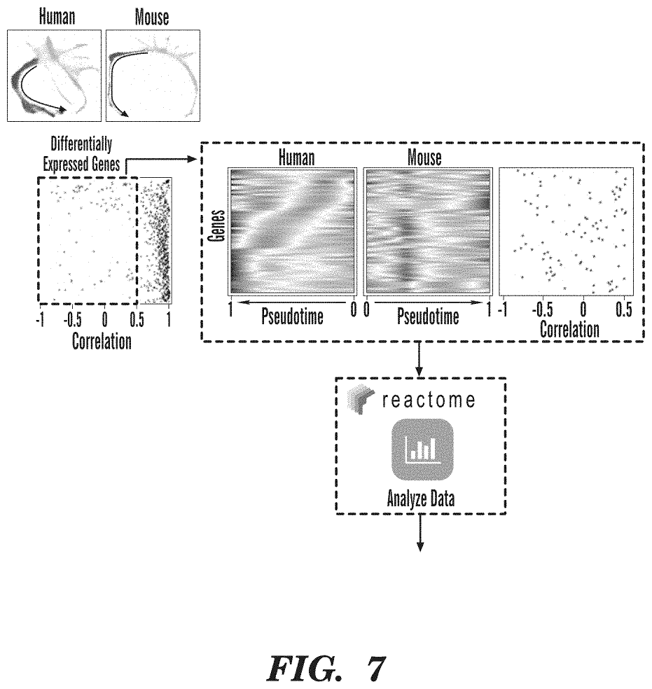

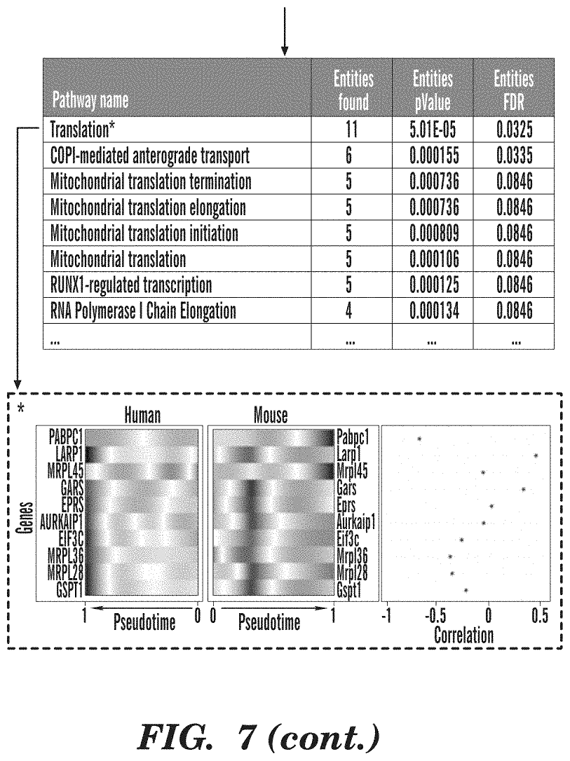

A question of practical interest for modelling human disease is the relationship between human and mouse hematopoiesis. While cell surface markers used to isolate HSPC sub-populations are known to differ between the two species, scRNA-Seq provides an opportunity to link population structure using whole-transcriptome information. The scRNA-Seq map of the human Lin− population was compared to that of mouse HSPCs, using published data on c-Kit⁺ mouse bone marrow progenitors. This analysis unveiled a strong similarity between the hierarchical structure of hematopoiesis in the two organisms, which show an identical branching hierarchy of cell states (FIG. 3-1A versus FIGS. 2-1D, 3-2B, 3-2C, and 7). Furthermore, comparing branch-specific gene signatures identified that the vast majority of gene orthologues in the erythroid branch were equivalently expressed in human and mouse (FIG. 3-1B). Recently, it was shown that erythroid progenitors in the mouse can be classified as ‘early,’ which uniquely give rise to burst-forming units (BFU-e) and are marked by Trib2; and as “committed”, which give rise to colony-forming units (CFU-e) and express Cart. Notably, the same progression was observed from TRIB2-expressing to CA2-expressing erythroid progenitors (FIG. 3-1C) suggesting the existence of the same two precursors subclasses in humans. In this regard, the data also confirmed and expanded the information on the divergence of human and mouse erythropoiesis (FIG. 3-1C). Of note, when analyzing the human-mouse orthologues that are differently expressed along the erythroid branch, the most significant distinction is the expression of genes involved in the molecular apparatus supporting protein translation (FIG. 7). This difference in the expression of the machinery of ribosome biogenesis during erythropoiesis could explain why mouse models of red blood cells disorders caused by a partial loss of ribosomal function, such as Diamond-Blackfan anemia, are not able to recapitulate the human phenotype.

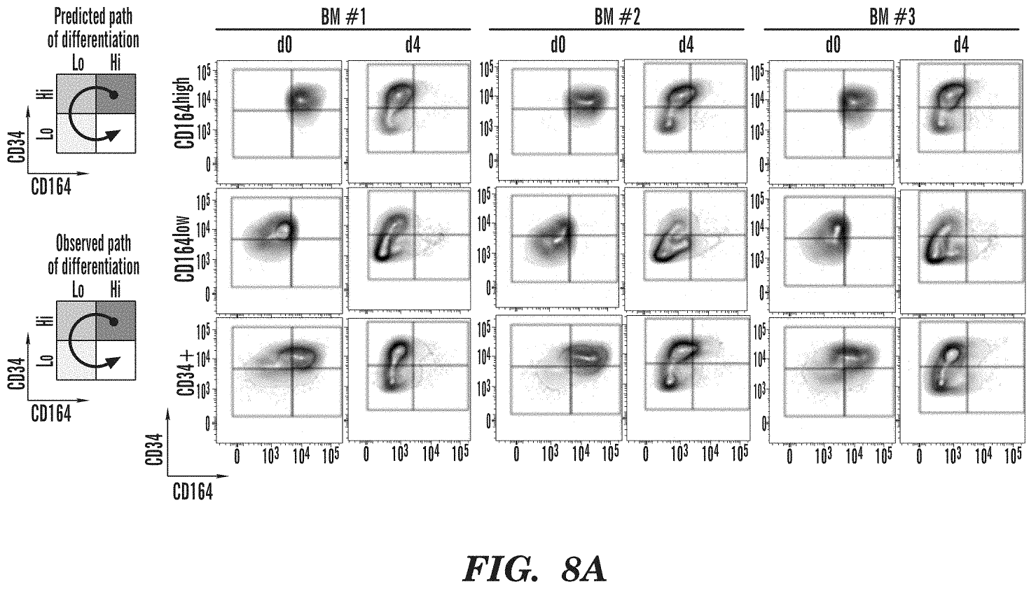

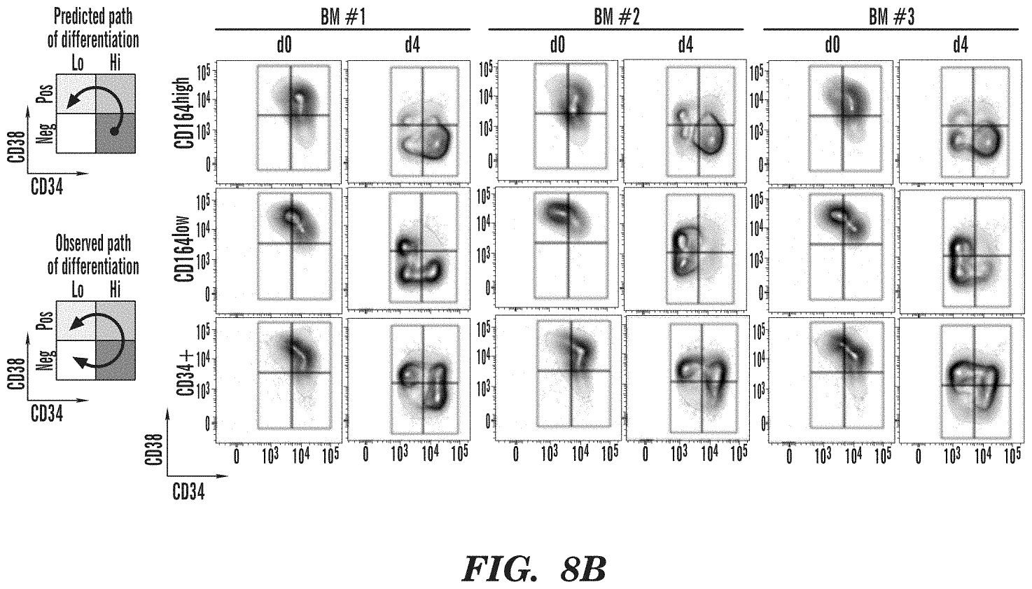

Experiments were undertaken to determine whether advantage could be taken of the data to rationally select a cell surface marker to fractionate human HSPCs for transplantation and gene therapy (FIGS. 4-1A to 4-1K). To date, the CD38 antigen has served to negatively enrich for the primitive progenitors for transplantation. Yet this marker suffers three shortcomings and thus motivated a search for an alternative. First, there is no consensus on the gating strategy to be used for CD38 expression to define CD38− primitive cells, resulting in variable efficiencies of progenitor cell enrichment. Second, in strategies proposing a CD38⁻ cell selection for transplantation, CD38⁺ myeloid progenitor cells (CMP and GMP) must be provided separately to support short-term granulopoiesis in conditioned neutropenic patients. Third, as shown herein expression of CD38 is rapidly lost in culture upon cytokine exposure (FIG. 8B), meaning that the viability and composition of early progenitors cannot be verified in transplantation products after in vitro expansion using the CD38− cytometric gating. The cell surface antigen CD164 overcomes all three of these shortcomings, and can advantageously be used to select human HSPCs for transplantation and gene therapy.

The CD164 gene encodes for a membrane-associated sialomucin, endolyn, whose function is that of an adhesion receptor. Until now the expression of CD164 in the currently defined HSPC subpopulations and upon in vitro manipulation of CD34+ cells was not appreciated.

In the scRNA-Seq data, in testing for enrichment of transcripts encoding all surface antigens in early progenitors (FIG. 1-1G), CD164 emerged as the surface marker gene whose expression displayed the most pronounced difference in early vs late progenitors. By contrast, neither CD38 nor CD90 (common marker used for identification of primitive HSPCs) transcripts strongly discriminated between early versus late stages of blood cell fate commitment. Although mRNA abundances do not necessarily correlate with protein abundance, it was found that the CD34+ population can be split into two sub-fractions on the basis of two clearly distinct levels of CD164 transcript abundances (FIG. 1-1H), which tracked fractionation by CD164 antibody-based sorting. The CD164 RNA is selectively expressed at high level not only in CD38⁻ multipotent progenitors, but also in CD90⁺ precursors (which in humans comprise both HSC and early common myeloid progenitors (CMP)), in the most primitive fraction of MEP and to a lesser extent, in multi-lymphoid progenitors (MLP) (FIG. 1-1G). During later stages of commitment, the CD164 mRNA and protein surface expressions levels begin to diverge (e.g., in the CD34−CD164^{high} erythroid-committed cells).

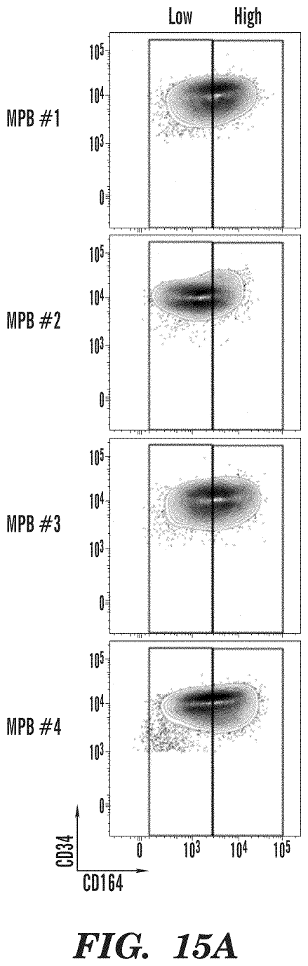

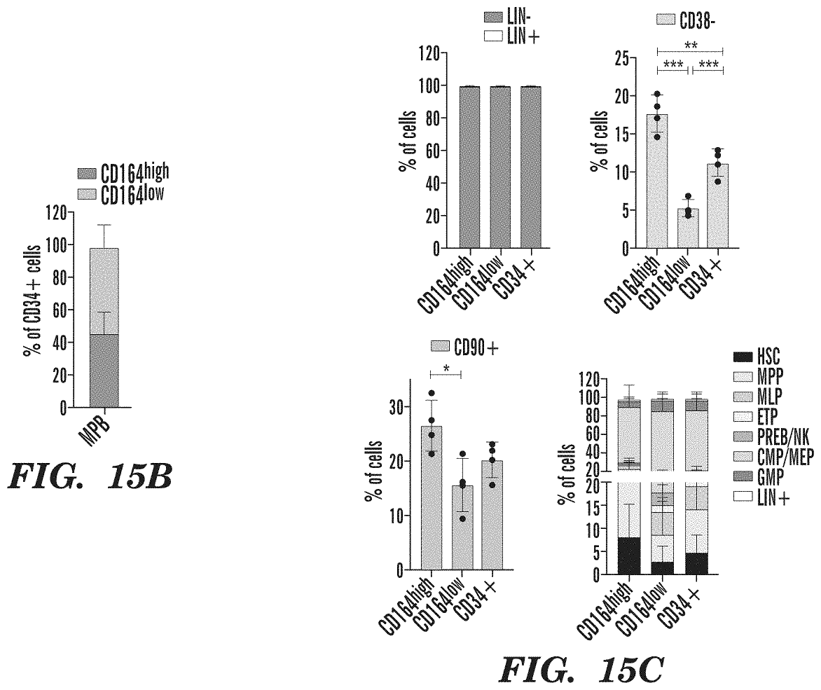

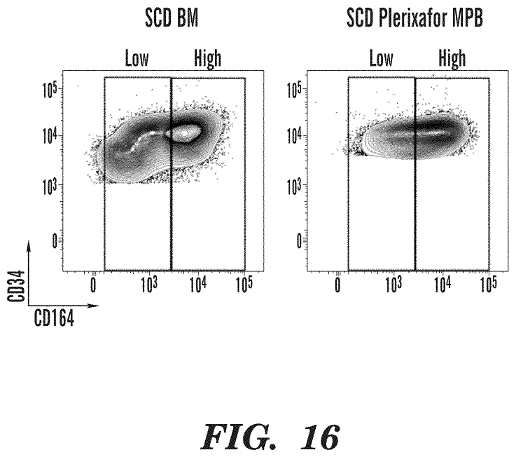

To investigate the utility of CD164 role in fractionating early hematopoietic progenitors, a series of immunophenotypic and functional assays was performed on human BM CD34+ cells (FIG. 4-1A). In line with scRNA-Seq results, a cytometric analysis combining anti-CD164 antibody with the other classical HSPCs markers, confirmed that the CD34+ population contains two clearly distinct fractions of CD164^{high} and CD164^{low} expressing cells, the first of which was highly enriched in cells with cytometric markers of primitive progenitors, MEPs and early CMPs and, notably, was almost entirely depleted of preB-NK and Lin+ cells (FIGS. 4-1B to 4-1E and FIG. 9A and Table 3). Importantly, this differential composition between CD164^{high} and CD164^{low} populations in the human BM is not merely owing to the differences in the relative CD34 surface expression or in the Lin+ cell content. Indeed, the same were obtained analyzing CD34+ cells from G-CSF− and plerixafor-mobilized peripheral blood where the CD34 expression is uniform in both CD164^{high} and CD164^{low} cell fractions and where the contribution of the Lin+ population is negligible (FIGS. 15A-15C and 16, Table 4). The method of moment estimation of each HSPC subpopulation proportion with related standard deviation are provided in Tables 3 and 4. The comparison of proportion estimates in the CD164^{high} and CD164^{low} fractions, and CD34+ cells have been calculated by means of the Student's t-test, under different variance hypothesis. To date, the literature reports only the results of a clonogenic assay as a test of the in vitro differentiation potential of CD34+CD164+ cells (Zannettino et al., Blood 92, 2613-28 (1998)).

TABLE 3
Statistical analysis on the HSPC bar graphs of FIG. 4-1D
BM #1, 2, 3, 4, 5, 6, 7, 8, 9

| | | CD164^{high} | | CD164^{low} | | | | |
|---|---|---|---|---|---|---|---|---|
| | | Parameter | Parameter | Parameter | Parameter | | | |
| | Parameter | Est. | Est. S.E. | Est. | Est. S.E. | T-test | | |
| Subpopulation | [P(Subpop)] | [MoM] | [MoM] | [MoM] | [MoM] | P-value | CD164^{high} | CD164^{low} |
| HSC | pi:HSC | 3.420E−02 | 2.270E−02 | 7.590E−04 | 1.283E−03 | 2.21E−03 | ▴ | ▾ |
| MPP | pi:MPP | 5.441E−02 | 4.562E−02 | 6.130E−03 | 7.878E−03 | 1.31E−02 | ▴ | ▾ |
| MLP | pi:MLP | 8.778E−03 | 4.213E−03 | 5.018E−03 | 5.737E−03 | 1.34E−01 | — | — |
| ETP | pi:ETP | 1.935E−02 | 7.883E−03 | 2.042E−02 | 7.231E−03 | 7.67E−01 | — | — |
| PREB/NK | pi:PREB | 1.999E−02 | 6.904E−03 | 1.094E−01 | 4.410E−02 | 2.65E−04 | ▾ | ▴ |
| CMP/MEP | pi:CMP/MEP | 4.849E−01 | 4.284E−02 | 1.081E−01 | 7.906E−02 | 2.16E−08 | ▴ | ▾ |
| GMP | pi:GMP | 2.969E−01 | 5.665E−02 | 3.205E−01 | 4.973E−02 | 3.63E−01 | — | — |
| LINp | pi:LINp | 8.148E−02 | 3.837E−02 | 4.297E−01 | 1.263E−01 | 1.79E−05 | ▾ | ▴ |

| | | CD164^{high} | | CD34+ | | | | |
|---|---|---|---|---|---|---|---|---|
| | | Parameter | Parameter | Parameter | Parameter | | | |
| | Parameter | Est. | Est. S.E. | Est. | Est. S.E. | T-test | | |
| Subpopulation | [P(Subpop)] | [MoM] | [MoM] | [MoM] | [MoM] | P-value | CD164^{high} | CD34 |
| HSC | pi:HSC | 3.420E−02 | 2.270E−02 | 2.510E−02 | 1.906E−02 | 3.71E−01 | — | — |
| MPP | pi:MPP | 5.441E−02 | 4.562E−02 | 3.750E−02 | 2.945E−02 | 3.66E−01 | — | — |
| MLP | pi:MLP | 8.778E−03 | 4.213E−03 | 8.361E−03 | 4.455E−03 | 8.41E−01 | — | — |
| ETP | pi:ETP | 1.935E−02 | 7.883E−03 | 1.975E−02 | 7.595E−03 | 9.14E−01 | — | — |
| PREB/NK | pi:PREB | 1.999E−02 | 6.904E−03 | 4.238E−02 | 1.808E−02 | 5.79E−03 | ▾ | ▴ |
| CMP/MEP | pi:CMP/MEP | 4.849E−01 | 4.284E−02 | 3.818E−01 | 4.371E−02 | 1.18E−04 | ▴ | ▾ |
| GMP | pi:GMP | 2.969E−01 | 5.665E−02 | 3.086E−01 | 3.643E−02 | 6.12E−01 | — | — |
| LINp | pi:LINp | 8.148E−02 | 3.837E−02 | 1.765E−01 | 3.925E−02 | 8.87E−05 | ▾ | ▴ |

TABLE 3
Statistical analysis on the HSPC bar graphs of FIG. 4-1D (Cont'd)

| | | CD164^{low} | | CD34+ | | | | |
|---|---|---|---|---|---|---|---|---|
| | | Parameter | Parameter | Parameter | Parameter | | | |
| | Parameter | Est. | Est. S.E. | Est. | Est. S.E. | T-test | | |
| Subpopulation | [P(Subpop)] | [MoM] | [MoM] | [MoM] | [MoM] | P-value | CD164^{low} | CD34 |
| HSC | pi:HSC | 7.590E−04 | 1.283E−03 | 2.510E−02 | 1.906E−02 | 4.98E−03 | ▾ | ▴ |
| MPP | pi:MPP | 6.130E−03 | 7.878E−03 | 3.750E−02 | 2.945E−02 | 1.28E−02 | ▾ | ▴ |
| MLP | pi:MLP | 5.018E−03 | 5.737E−03 | 8.361E−03 | 4.455E−03 | 1.88E−01 | — | — |
| ETP | pi:ETP | 2.042E−02 | 7.231E−03 | 1.975E−02 | 7.595E−03 | 8.50E−01 | — | — |
| PREB/NK | pi:PREB | 1.094E−01 | 4.410E−02 | 4.238E−02 | 1.808E−02 | 1.56E−03 | ▴ | ▾ |
| CMP/MEP | pi:CMP/MEP | 1.081E−01 | 7.906E−02 | 3.818E−01 | 4.371E−02 | 7.42E−07 | ▾ | ▴ |
| GMP | pi:GMP | 3.205E−01 | 4.973E−02 | 3.086E−01 | 3.643E−02 | 5.71E−01 | — | — |
| LINp | pi:LINp | 4.297E−01 | 1.263E−01 | 1.765E−01 | 3.925E−02 | 2.24E−04 | ▴ | ▾ |

TABLE 4
Statistical analysis on the HSPC bar graphs of FIG. 15C
MPB #1, 2, 3, 4

| | | CD164^{high} | | CD164^{low} | | | | |
|---|---|---|---|---|---|---|---|---|
| | | Parameter | Parameter | Parameter | Parameter | | | |
| | Parameter | Est | Est. S.E. | Est. | Est. S.E. | T-test | | |
| Subpopulation | [P(Subpop)] | [MoM] | [MoM] | [MoM] | [MoM] | P-value | CD164^{high} | CD164^{low} |
| HSC | pi:HSC | 5.166E−02 | 2.289E−02 | 1.233E−02 | 2.596E−03 | 9.50E−02 | — | — |
| MPP | pi:MPP | 9.830E−02 | 2.687E−02 | 2.739E−02 | 8.725E−03 | 3.45E−02 | ▴ | ▾ |
| MLP | pi:MLP | 2.815E−02 | 2.133E−02 | 1.324E−02 | 7.789E−03 | 3.52E−01 | — | — |
| ETP | pi:ETP | 6.949E−03 | 9.757E−03 | 1.073E−02 | 8.859E−03 | 6.46E−01 | — | — |
| PREB/NK | pi:PREB | 1.400E−02 | 1.266E−02 | 2.975E−02 | 1.803E−02 | 2.91E−01 | — | — |
| CMP/MEP | pi:CMP/MEP | 7.487E−02 | 5.858E−02 | 1.235E−01 | 7.267E−02 | 4.20E−01 | — | — |
| GMP | pi:GMP | 7.209E−01 | 5.484E−02 | 7.759E−01 | 8.693E−02 | 4.15E−01 | — | — |
| LINp | pi:LINp | 5.199E−03 | 3.574E−03 | 7.126E−03 | 5.662E−03 | 6.49E−01 | — | — |

| | | CD164^{high} | | CD34+ | | | | |
|---|---|---|---|---|---|---|---|---|
| | | Parameter | Parameter | Parameter | Parameter | | | |
| | Parameter | Est. | Est. S.E. | Est. | Est S.E. | T-test | | |
| Subpopulation | [P(Subpop)] | [MoM] | [MoM] | [MoM] | [MoM] | P-value | CD164^{high} | CD34 |
| HSC | pi:HSC | 5.166E−02 | 2.289E−02 | 3.219E−02 | 1.480E−02 | 2.94E−01 | — | — |
| MPP | pi:MPP | 9.830E−02 | 2.687E−02 | 6.261E−02 | 1.798E−02 | 1.39E−01 | — | — |
| MLP | pi:MLP | 2.815E−02 | 2.133E−02 | 1.897E−02 | 1.081E−02 | 5.54E−01 | — | — |
| ETP | pi:ETP | 6.949E−03 | 9.757E−03 | 7.226E−03 | 9.422E−03 | 9.73E−01 | — | — |
| PREB/NK | pi:PREB | 1.400E−02 | 1.266E−02 | 1.961E−02 | 1.139E−02 | 5.99E−01 | — | — |
| CMP/MEP | pi:CMP/MEP | 7.487E−02 | 5.858E−02 | 1.039E−01 | 7.494E−02 | 6.27E−01 | — | — |
| GMP | pi:GMP | 7.209E−01 | 5.484E−02 | 7.495E−01 | 6.642E−02 | 5.97E−01 | — | — |
| LINp | pi:LINp | 5.199E−03 | 3.574E−03 | 5.999E−03 | 3.254E−03 | 7.89E−01 | — | — |

TABLE 4
Statistical analysis on the HSPC bar graphs of FIG. 15C (Cont'd)

| | | CD164^{low} | | CD34+ | | | | |
|---|---|---|---|---|---|---|---|---|
| | | Parameter | Parameter | Parameter | Parameter | | | |
| | Parameter | Est. | Est. S.E. | Est. | Est. S.E. | T-test | | |
| Subpopulation | [P(Subpop)] | [MoM] | [MoM] | [MoM] | [MoM] | P-value | CD164^{low} | CD34 |
| HSC | pi:HSC | 1.233E−02 | 2.596E−03 | 3.219E−02 | 1.480E−02 | 1.42E−01 | — | — |
| MPP | pi:MPP | 2.739E−02 | 8.725E−03 | 6.261E−02 | 1.798E−02 | 5.80E−02 | — | — |
| MLP | pi:MLP | 1.324E−02 | 7.789E−03 | 1.897E−02 | 1.081E−02 | 5.01E−01 | — | — |
| ETP | pi:ETP | 1.073E−02 | 8.859E−03 | 7.226E−03 | 9.422E−03 | 6.64E−01 | — | — |
| PREB/NK | pi:PREB | 2.975E−02 | 1.803E−02 | 1.961E−02 | 1.139E−02 | 4.65E−01 | — | — |
| CMP/MEP | pi:CMP/MEP | 1.235E−01 | 7.267E−02 | 1.039E−01 | 7.494E−02 | 7.61E−01 | — | — |
| GMP | pi.GMP | 7.759E−01 | 8.693E−02 | 7.495E−01 | 6.642E−02 | 6.99E−01 | — | — |
| LINp | pi:LINp | 7.126E−03 | 5.662E−03 | 5.999E−03 | 3.254E−03 | 7.83E−01 | — | — |

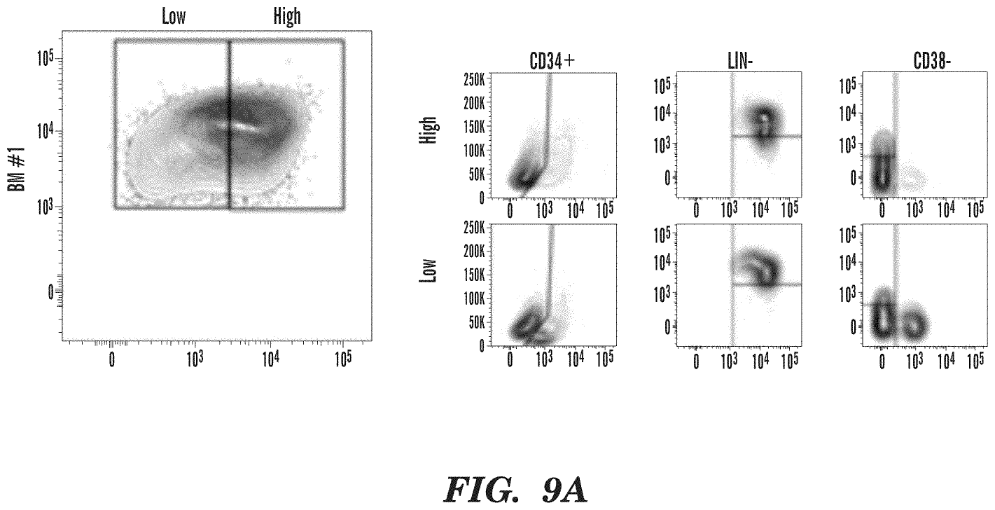

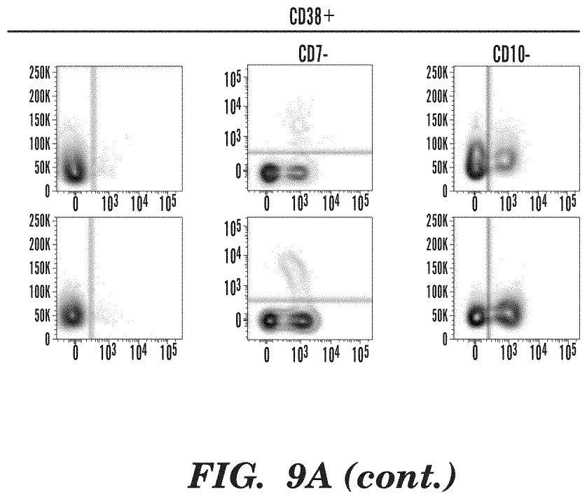

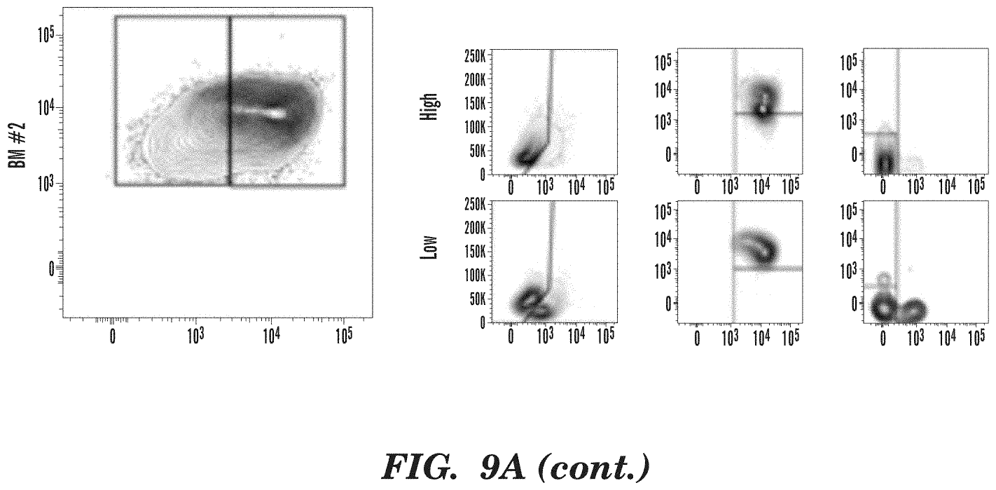

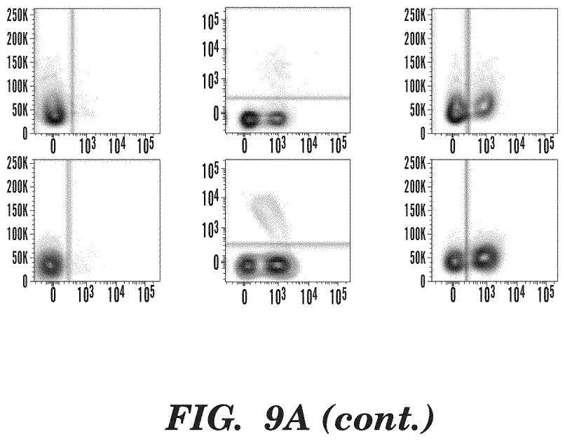

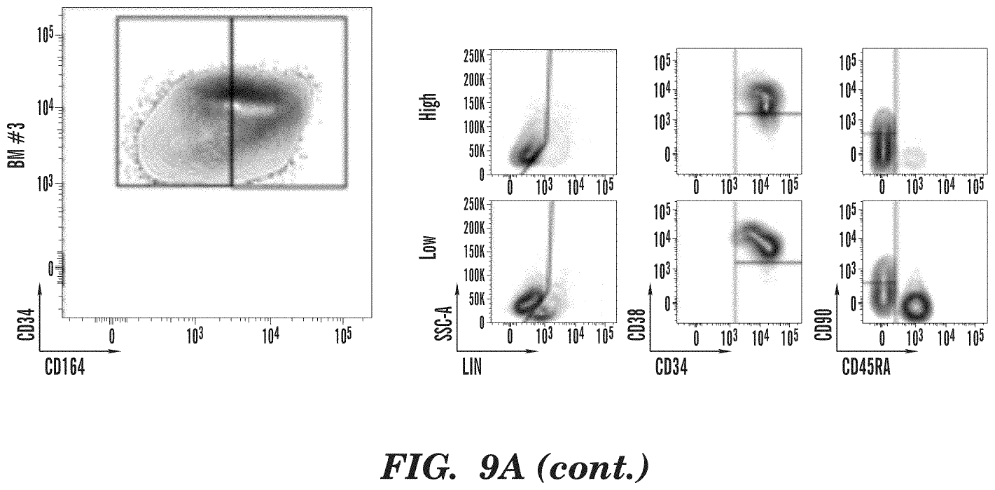

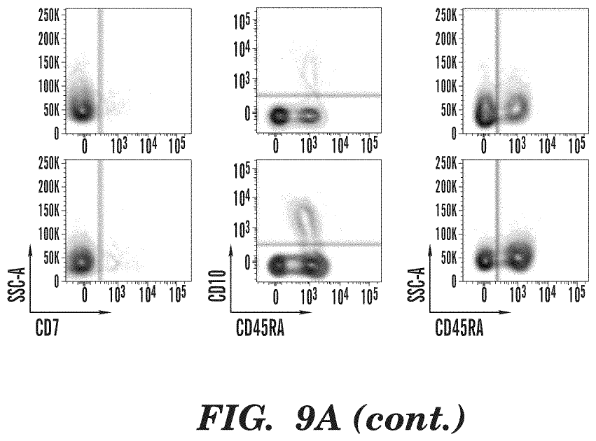

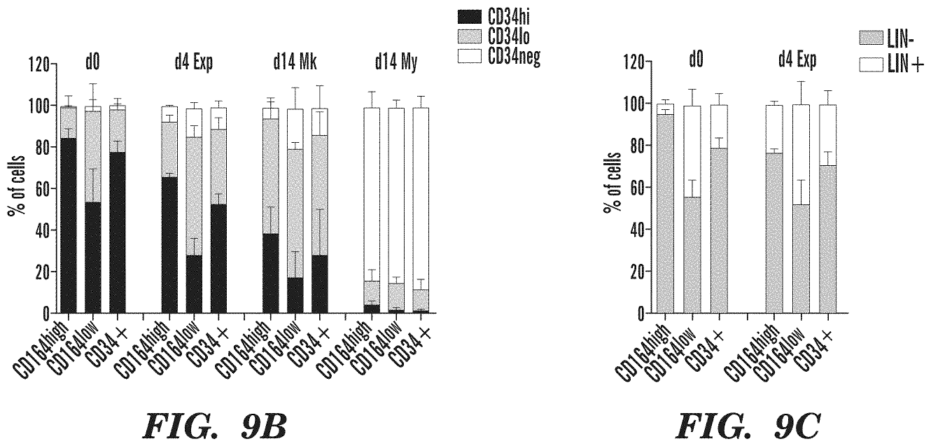

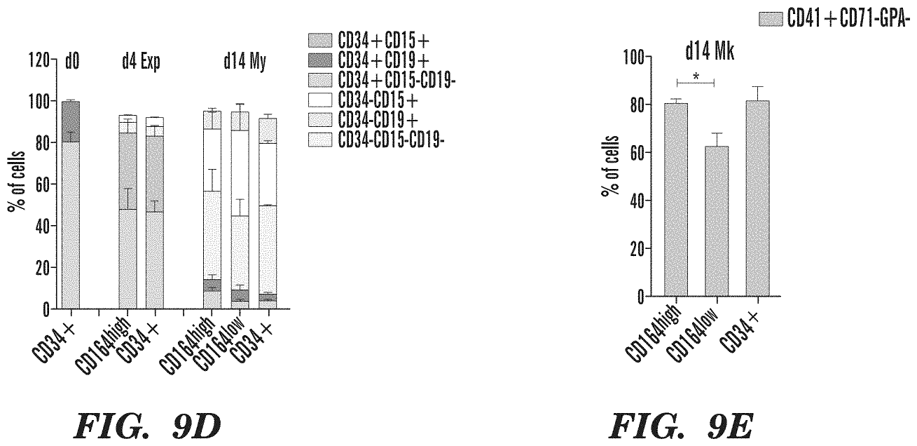

To integrate these data, a set of functional tests was carried out on FACS-sorted CD34+CD164^{high} and CD34+CD164^{low} cells from the BM of three healthy donors (FIG. 4-1F to 4-1H). The CD34+CD164^{high}population displayed a superior in vitro differentiation potential as compared to the CD34+ CD164^{low} fraction and even to the total CD34+ population, showing higher rate of colonies generation and of expansion not only in Myeloid (MY) but also in MK differentiating conditions (FIGS. 4-1F and 4-1H). Cytometric analysis of differentiation states after culture confirmed the more primitive nature of CD34+CD164^{high}cells (FIG. 4-1H, 4-1I, and FIGS. 9B-9E). Lastly, the CD34+CD164^{high} cells expanded more rapidly in culture conditions used in clinical gene therapy for in vitro stem cell enrichment prior to autologous transplantation (FIG. 4-1G). Importantly, in this context, CD164 allows, as compared to CD38, a more robust cytometric estimation of the primitive progenitor content upon in vitro manipulation of CD34+ cells, since its loss of expression coincides with the progressive cell differentiation upon cytokine exposure (FIGS. 8A and 8B). This is a major advantage over the use of the classical CD38 marker whose expression dynamics were instead not consistent with the expected phenotype changes of differentiating cells. These in vitro functional assays provide a proof of principle of the biological significance and translatability of the information contained in these scRNA-Seq maps.

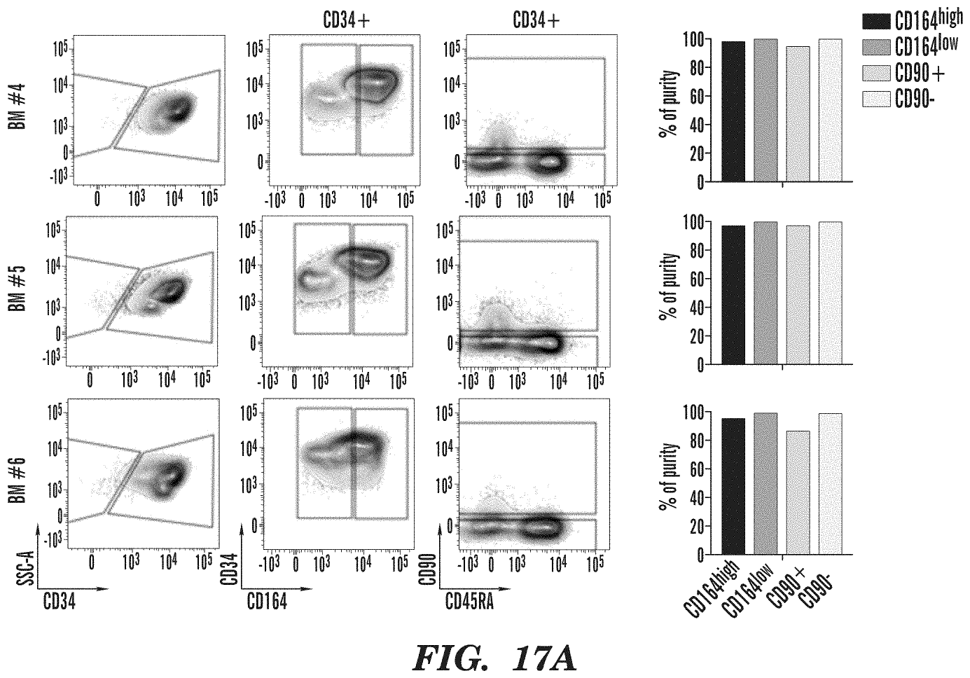

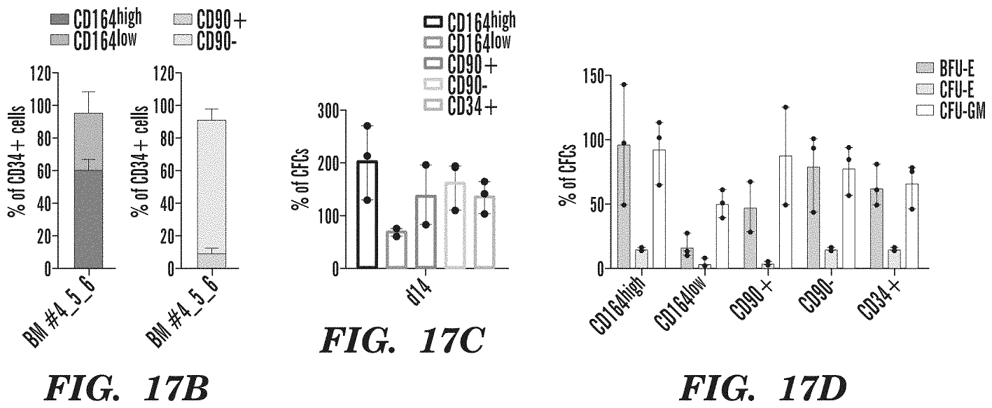

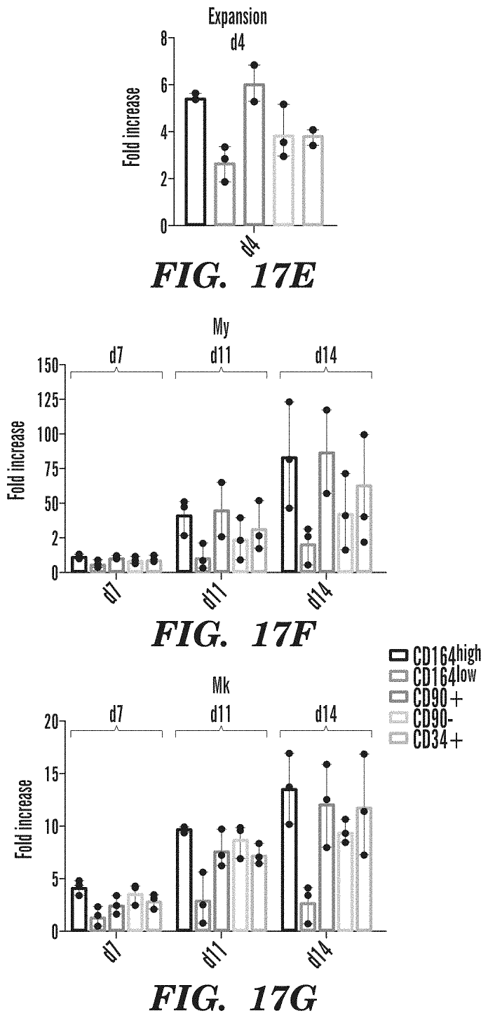

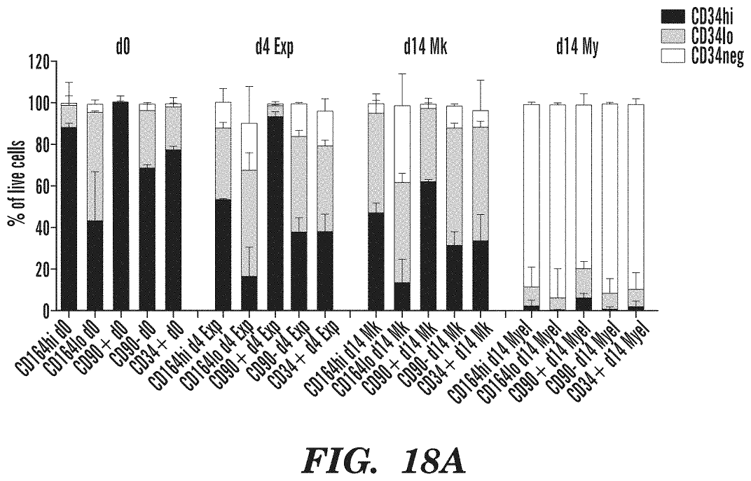

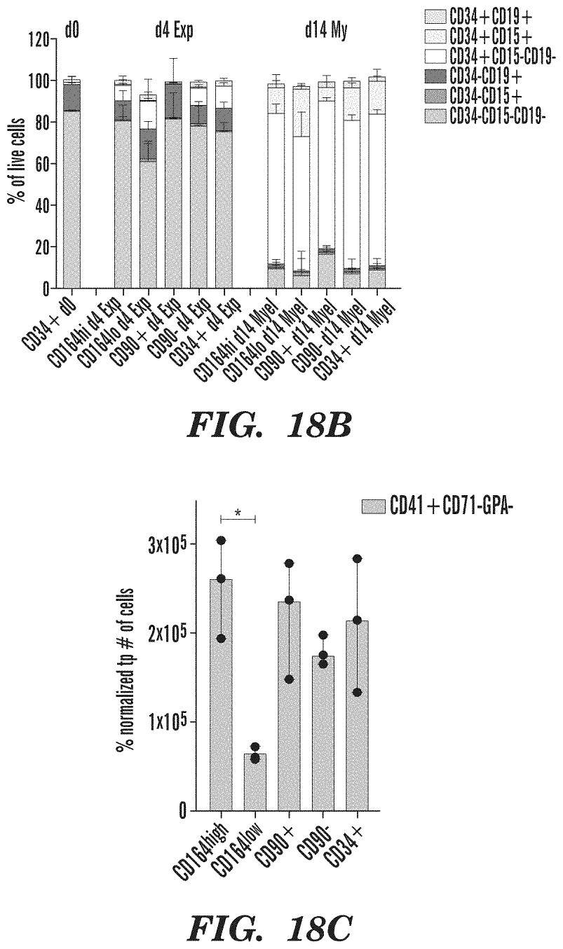

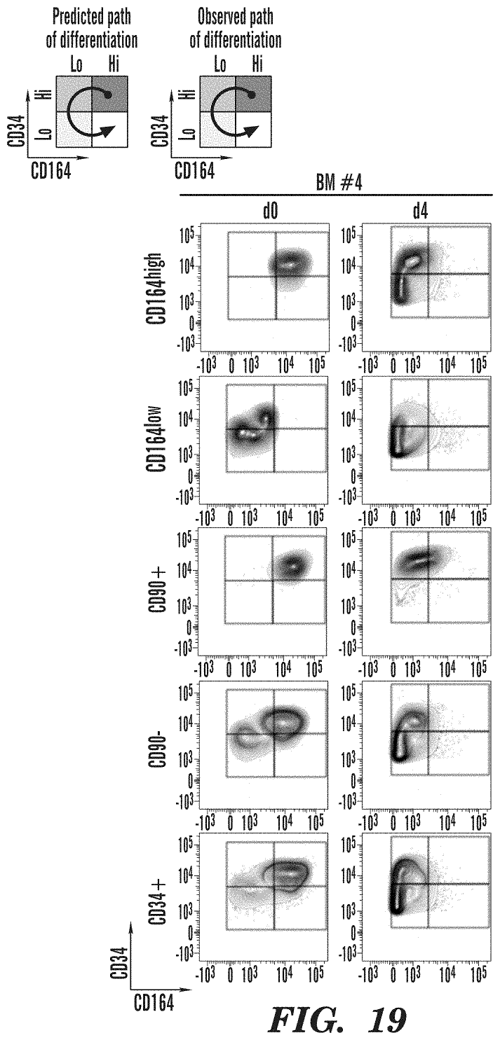

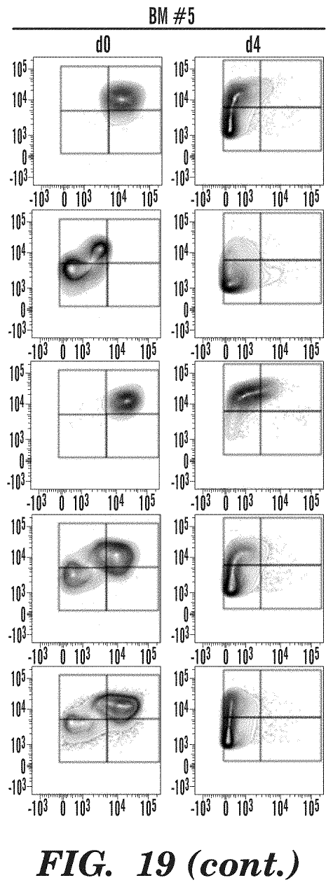

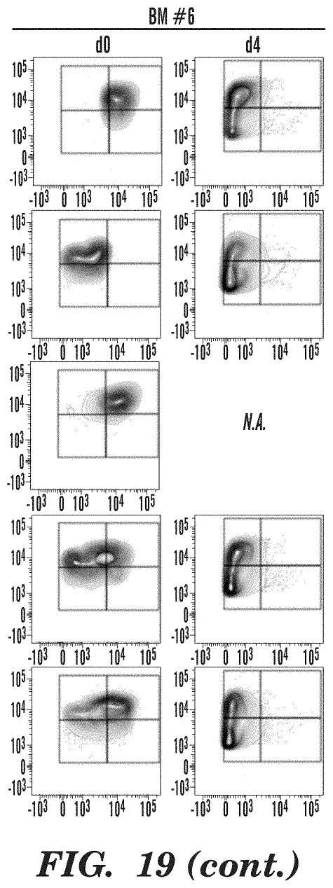

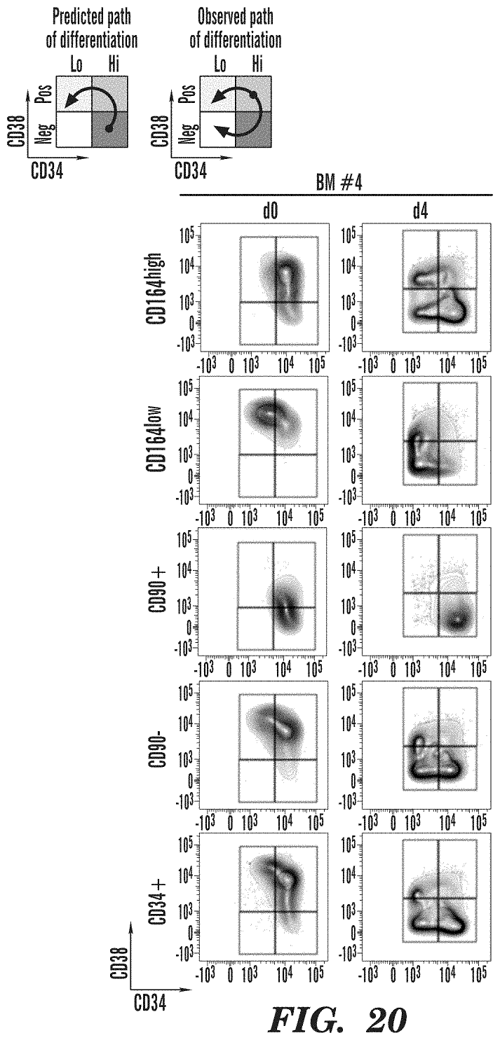

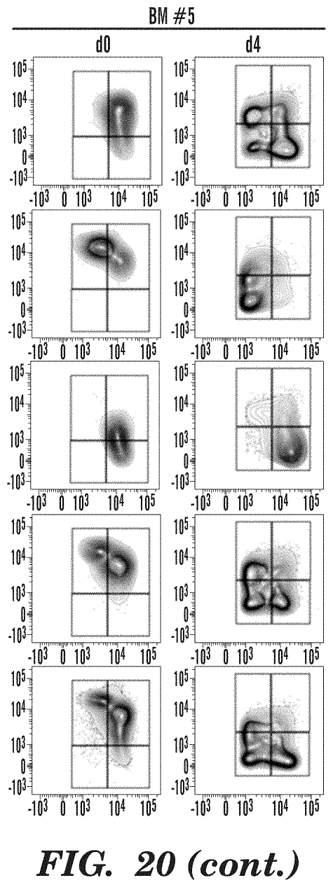

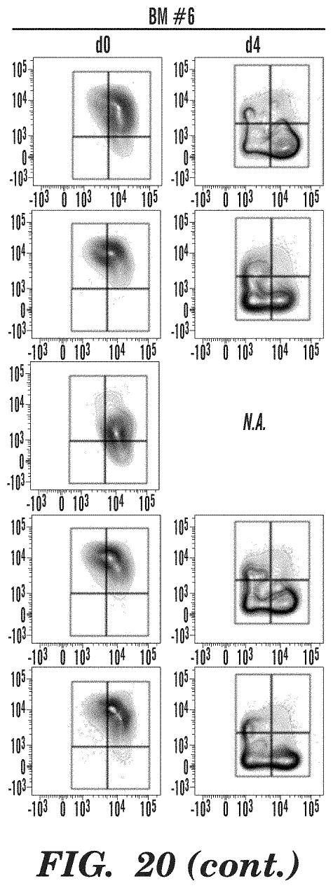

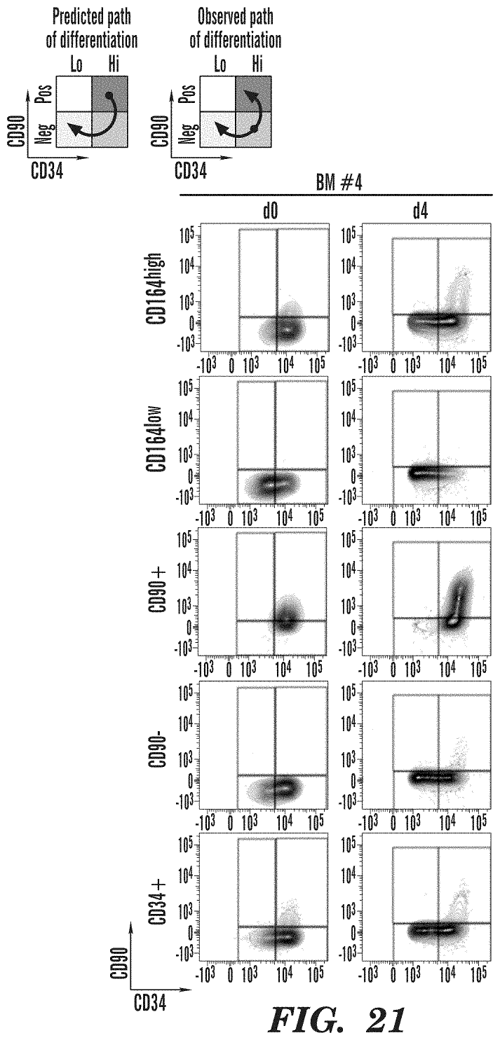

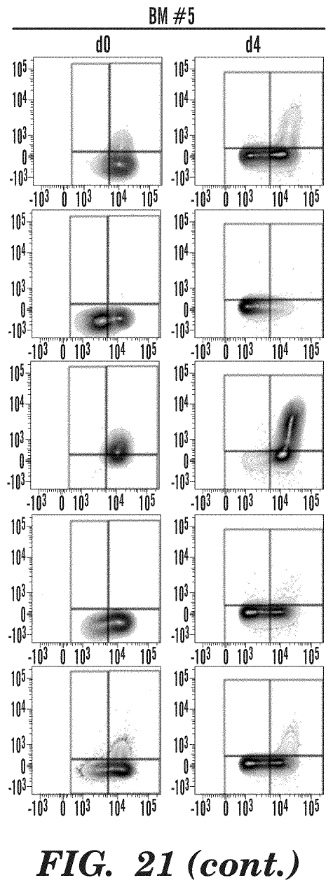

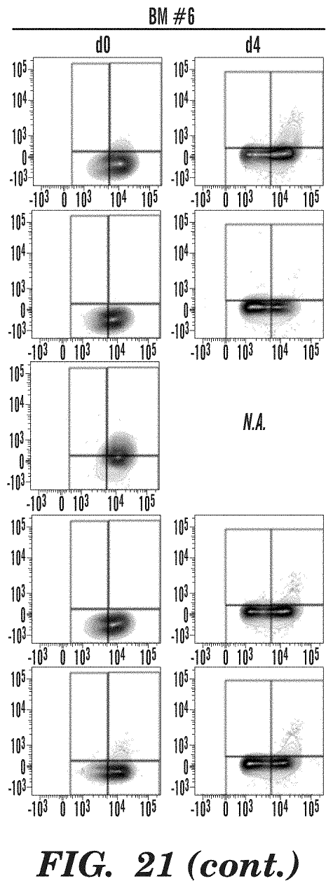

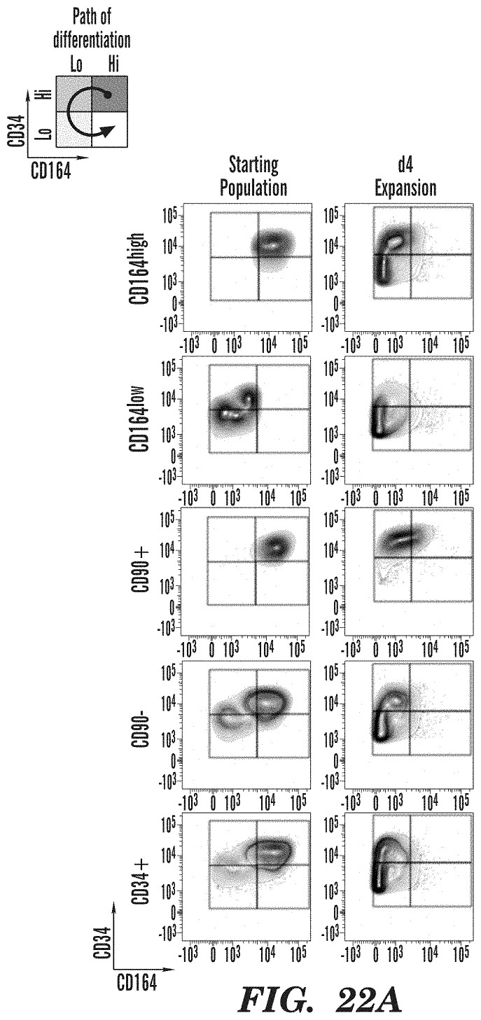

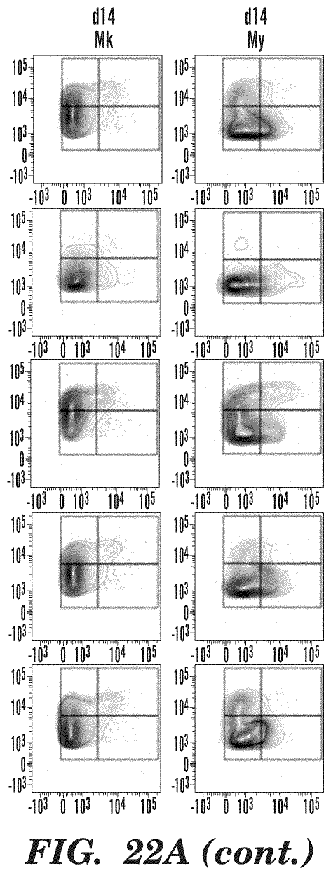

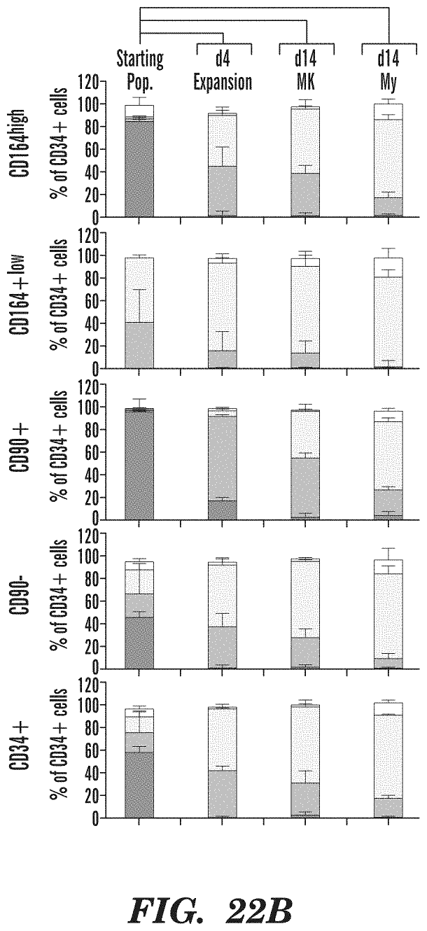

Another key surface marker used for the identification of stem/multipotent vs committed progenitor is CD90. Additional differentiation assays were conducted on three healthy donors comparing the performance of FACS-sorted CD34+CD90+ cells to CD34+CD164^{high} population. The results displayed in FIGS. 17A-17G and 18A-18C show that the CD34+CD164^{high} fraction has a much higher discriminatory potential, as compared with the CD34+CD90+ selection, for cells capable of growing in myeloid- and MK-differentiating conditions and for clonogenic progenitors (FIGS. 17G and 18C). Furthermore, as in the case of CD38, the CD90 marker presented inconsistent expression dynamics in culture, being upregulated (and not downregulated) upon cell differentiation (FIGS. 19-21), again pointing to the superior performance of CD164 in allowing a more reliable evaluation of the stem cell content of in vitro manipulated CD34+ cell products (FIGS. 22A and 22B).

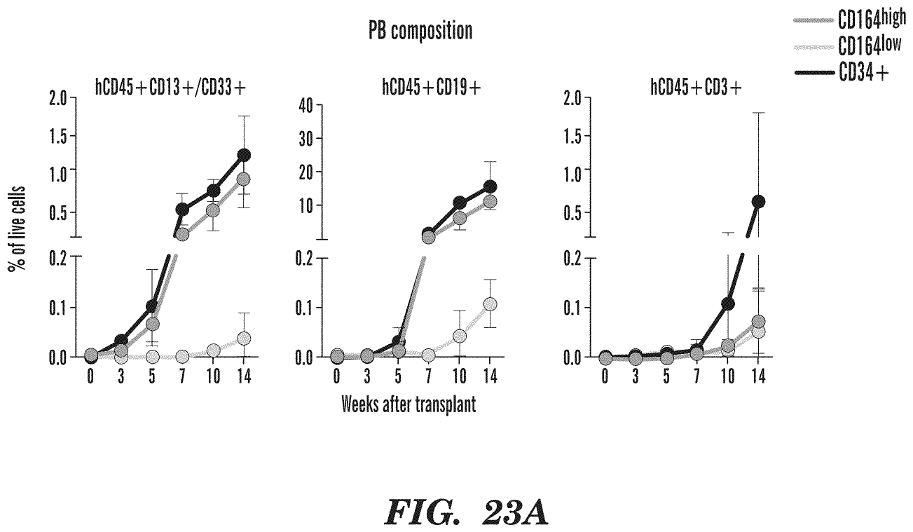

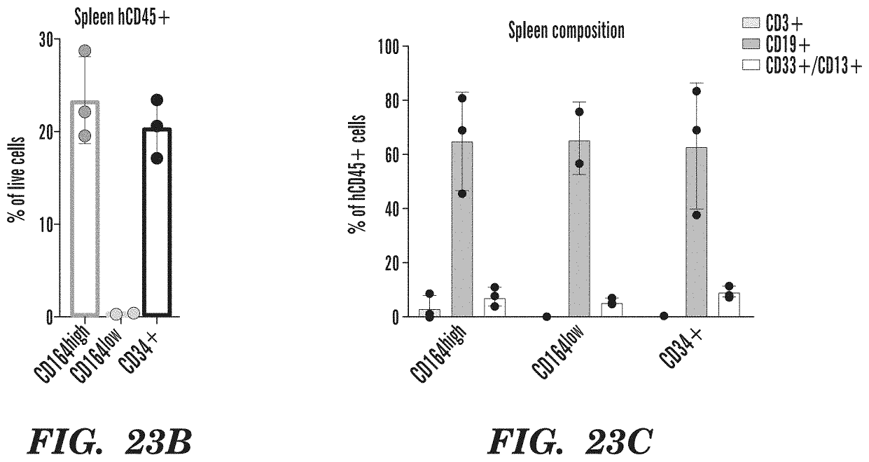

The CD34+CD164^{high} population contains both multipotent progenitors and early CMP. On the basis of the model of hematopoietic reconstitution emerging from clonal tracking data in humans, it was reasoned that the CD34+CD164^{high} fraction might constitute a suitable self-sufficient cell product for transplantation that would not require the co-infusion of other cells to support recovery from neutropenia and early myelopoiesis. To test this hypothesis, CD34+CD164^{high} vs CD34+CD164^{low} populations were sorted and transplanted into NOD.Cg-Kit^{W-41J}Tyr+Prkdc^{scid}Il2rg^{tm1Wjl}/ThomJ (NBSGW) mice (FIGS. 4-1I to 4-1K and 23A-23C). The results confirmed that the CD34+CD164^{high} cell product is capable of sustaining both the early and late phases of hematopoietic reconstitution, whereas the CD34+CD164^{low} population did not have a role in blood cell production at either stage, making its use in transplantation virtually dispensable. Remarkably, and in line with this observation, the dynamics and size of human lymphoid and myeloid cells output in the mice infused with FACS-sorted CD34+CD164^{high} cells was comparable to the mice infused with CD34+ cells, despite the latter receiving twice the amount of cells. Overall, data presented herein clearly highlight the biological relevance of the CD164 gene in early hematopoiesis, reviving the use of this marker for the study of human HSPC and setting the basis for exploring the potential use of the CD34+CD164^{high} fraction in clinical transplantation and gene therapy, where there is a high demand for reducing the production costs for genetic engineering.

The results reported herein above, were obtained using the following methods and materials.

### Cell Preparation.

Bone marrow (BM) samples were collected from adult healthy donors at Children's Hospital in Boston with the approval of the Committee on Clinical Investigations Children's Hospital Boston and consent from the subjects under the protocol #09-04-0167. Mononuclear cells (MNCs) were isolated using Ficoll-Hypaque gradient separation (Lymphoprep, STEMCELL Technologies). CD34+ cells were purified from MNCs with the human anti-CD34 MicroBeads Isolation Kit (Miltenyi Biotec) according to the manufacturer's specifications or were purchased from commercial sources (AllCells).

### Cell Sorting and Immunophenotyping.

Seven HSPC sub-populations were purified from the CD34+ fraction of a healthy donor BM cells through a two-step four-way sorting using FACSAria II (BD Biosciences) and processed to generate the transcriptome network in FIGS. 1-1A to 1-1H. The following combinations of cell surface markers were used to identify and separate the HSPC subsets. Hematopoietic stem cells (HSC): Lin−CD34+CD38−CD90+CD45RA−; multipotent progenitors (MPP): Lin−CD34+CD38−CD90−CD45RA−; multilymphoid progenitors (MLP): Lin−CD34+CD38−CD90−CD45RA+; pre-B lymphocytes/Natural Killer cells (PREB/NK): Lin−CD34+CD38+CD7−CD10+; megakaryocyte-erythroid progenitors (MEP): Lin−CD34+CD38+CD7−CD10−CD135−CD45RA−; common myeloid progenitors (CMP): Lin−CD34+CD38+CD7−CD10−CD135+CD45RA−; granulocyte-monocyte progenitors (GMP): Lin−CD34+CD38+CD7−CD10−CD135−CD45RA+.

For the generation of the transcriptome network in FIGS. 2-1A to 2-1F, four cell fractions were purified from a healthy donor BM MNCs through a four-way sorting using the following combinations of cell surface markers: Lin−CD34+CD164+; Lin−CD34^{low}CD164^{high}; Lin−CD34−CD164^{high}; Lin−CD34−CD164^{low}. CD71 was included to identify erythroid progenitors.

For in vitro functional assays, Lin−CD34+CD135− and Lin−CD34+CD135+ fractions were purified from the CD34+ cells of three independent BM through a two-way sorting. The cell subsets CD34+CD164^{high}and CD34+CD164^{low} were FACS-sorted from the CD34+ cells of nine independent BM. Of these, three BM were also used to purify CD34+CD90+ and CD34+CD90− cells.

For in vivo studies, CD34+CD164^{high} and CD34+CD164^{low} cells were FACS-sorted and purified from a pool of BM CD34+ cells from two additional healthy donors.

Immunophenotyping was performed on BM CD34+ cells labelled with CD164 in combination with HSPC subsets markers by using LSRFortessa (BD Biosciences). CD15 and CD19 were included to identify the lineage positive cells. Flow cytometry data were analyzed with FlowJo 10.2 (Tree Star). The antibodies were as follows: CD34 PB, CD38 PE/Cy5, CD90 APC, CD10 PE/Cy7, CD135 PE, Lin BV510 (CD3, CD14, CD16, CD19, CD20, CD56 BV510), CD15 BV510, CD164 (clone 67D2) PE, CD164 FITC, CD71 PerCP/Cy5.5, CD41 APC, CD19

PE/Cy7 (all Biolegend); CD45RA APC-H7, CD7 AF700, CD15 PE (all BD Biosciences), Glycophorin A (Miltenyi Biotec).

To characterize the basophils contribution in the human peripheral blood and upon in vitro differentiation, the gating strategy reported in FIG. 14A has been set using the following antibodies: CD34 PB (1:40, #343512), FceRIA APC (1:10, #334612), CD14 AF700 (1:10, #367114), CD19 PE/Cy7 (1:20, #302216), CD15 FITC (1:20, #555401), CCR3 PerCP/Cy5.5 (1:10, #310718), all Biolegend. IL5RA PE (1:10, #555902), BD Biosciences.

To evaluate the human cell engraftment in the murine peripheral blood, BM and spleen the antibodies were as follows: CD33 PE (1:40, #561816), CD13 PE (1:40, #555394), CD3 V500 (1:20, #561416), CD19 PE/Cy7 (1:80, #557835), mCD45 APC (1:100, #561018), mCD45 PE (1:100, #553081), 7-AAD (1:12, #559925), all BD Biosciences. CD45 PB (1:40, #368540) and CD41 APC (1:50, #303710) all Biolegend. Glycophorin A APC-Vio770 (1:22, #130-100-268), Miltenyi.

### In Vitro Functional Assays.

For the in vitro functional assays, two sub-populations CD34+CD164^{high} and CD34+CD164^{low} were FACS-sorted from the BM CD34+ cells of three different healthy donors. Unsorted CD34+ cells were also used as controls. Expansion and differentiation cultures of CD34+ cells or sort-purified CD164^{high} and CD164^{low} cells were performed with a starting cell number of 20,000 cells, unless otherwise indicated.

To test for basophil potential, cells were cultured in Iscove's Modified Dulbecco's medium (IMDM) containing 1% P/S/Glu, 20% FBS (Gemini) and supplemented with IL-3 (20 ng/ml), IL-5 (20 ng/ml), SCF (20 ng/ml), GM-CSF (50 ng/ml) for 3 days, whereas supplemented only with IL-3 (20 ng/ml) and IL-5 (20 ng/ml) from day 4 to day 14. Cells were counted on days 7,11,14. Fresh medium was added as needed, to keep the cell concentration at 0.5×10⁶/mL. At the end of the culture, cells were analyzed by flow cytometry for the basophil markers and mounted on cytospin preparation to define the presence of basophils by Giemsa staining.

Myeloid potential was evaluated in IMDM medium containing 1% P/S/Glu and 10% FBS (Gemini) and supplemented with IL-3 (60 ng/ml), SCF (300 ng/ml), IL-6 (60 ng/ml) for 2 weeks.

Cells were counted on days 7,11,14. Fresh medium was added as needed, to keep the cell concentration at 1×10⁶/mL. At the end of the culture, cells were analyzed by flow cytometry for immunophenotyping and lineage-positive markers CD15 and CD19, and for basophil markers.

Expansion culture was set up in serum-free CellGro SCGM medium (Cell Genix) containing 1% penicillin/streptomycin/glutamine (P/S/Glu, Lonza) and supplemented with FLT3-L (300 ng/ml), IL-3 (60 ng/ml), SCF (300 ng/ml), TPO (100 ng/ml) for 8 days. Cells were counted on days 4 and 8. Immunophenotyping and flow cytometric analysis for lineage positive markers CD15 and CD19 were performed at day 4.
Myeloid potential was evaluated in IMDM medium containing 1% P/S/Glu and 10% FBS (Gemini) and supplemented with IL-3 (60 ng/ml), SCF (300 ng/ml), IL-6 (60 ng/ml) for 2 weeks. Cells were counted on days 7,11,14. Fresh medium was added as needed, to keep the cell concentration at 1×10⁶/mL. At the end of the culture, cells were analyzed by flow cytometry for immunophenotyping and lineage positive markers CD15 and CD19. All growth factors and cytokines were purchased from Peprotech. Megakaryocyte potential was assessed in StemSpan SFEM II serum-free medium supplemented with StemSpan Megakaryocyte Expansion Supplement (STEMCELL Technologies) for 2 weeks. Cells were counted on days 7, 11, and 14. Fresh medium was added as needed, to keep the cell concentration at 1×10⁶/mL. Immunophenotyping and flow cytometric analysis for CD41, CD71 and Glycophorin A were performed at the end of the culture. From CD34+ cells and each freshly sorted CD164^{high} and CD164^{low} populations, 3,500 cells were plated with 2.4 ml of Methocult medium (H4434, STEMCELL Technologies) for 2 weeks. Erythroid (BFU-E or CFU-E) and granulocyte-macrophage (GM) colonies were scored from duplicate plates on day 14.

Megakaryocyte potential was assessed in StemSpan SFEM II serum-free medium supplemented with StemSpan Megakaryocyte Expansion Supplement (STEMCELL Technologies) for 2 weeks. Cells were counted on days 7, 11, 14. Fresh medium was added as needed, to keep the cell concentration at 1×10⁶/mL. Immunophenotyping and flow cytometric analysis for CD41, CD71, and Glycophorin A were performed at the end of the culture.

To test the clonogenic potential of sort-purified populations and CD34+ cells, single-sorted cells were deposited in 96-well plates in different culture conditions. Medium was added at day 7 and colonies were scored at day 14. From CD34+ cells and each freshly sorted CD164 ^{high} and CD164^{low} populations, the clonogenic potential was also assessed by seeding 3500 cells with 2.4 ml of Methocult medium (H4434, STEMCELL Technologies) for 2 weeks. Erythroid (BFU-E or CFU-E) and granulocyte-macrophage (GM) colonies were scored from duplicate plates on day 14.

Transplantation into Humanized Mouse Model

NOD.Cg-Kit^{W-41J}Tyr+Prkdc^{scid}Il2rg^{tm1Wjl}/ThomJ (NBSGW) mice were purchased from the Jackson Laboratory. All animal procedures were performed according to ethical regulations for animal testing and research, upon approval by the Institutional Care and Use Committee (IACUC) at the Dana-Farber Cancer Institute. Six-week-old mice were transplanted with human

HSPCs by tail injection without undergoing irradiation or other conditioning regimen. Mice were randomized in the following transplantation groups: sorted purified CD34+CD164^{high} (2.5×10⁵ cells/mouse) and CD34+CD164^{low} (2.5×10⁵ cells/mouse), immunomagnetic-selected CD34+ (5×10⁵ cells/mouse). For each sorted population, three mice were transplanted (four mice for the whole CD34+ population). Human cell engraftment was assessed by serial bleeding and immunophenotyping at 3, 5, 7, 10, 14 weeks post-transplant and in BM and spleen at sacrifice 16 weeks post-transplant.

### InDrops Single-Cell RNA Sequencing and Data Analysis.

Single-cell mRNA barcoding and preparation of libraries for sequencing were performed following the inDrop protocol previously described in Zillionis et al., with modifications as described for the “FACS subsets” samples in Tusi et al.. FACS-sorted subpopulations were individually processed for droplet barcoding (Tables 1A and 1B). Emulsions were split in aliquots each containing approximately 2500 single-cell barcoded transcriptomes. Libraries generated from each FACS-sorting were prepared in parallel and sequenced on Illumina NextSeq 500 using a NextSeq High Output 1×75 cycle kit. Raw sequencing data (FASTQ files) were processed using the previously described inDrops.py bioinformatics pipeline (available at github.com/indrops/indrops). Bowtie v.1.1.1 was used with parameter -e 100. All ambiguously mapped reads were excluded from analysis and reads were aligned to the Ensemble GRCh38.85 version of human genome.

### Cell Filtering and Data Normalization

Each library of sorted HSPC or sorted Lin−CD34/CD164 cells was processed according to the following procedure. Upon inspection of the histograms reporting the total reads per cell, barcodes were initially filtered according to a customized threshold in order to include only the most abundant ones (transcript counts threshold used for the sorted HSPC: HSC, 1000; CMP, 800; MEP, 1000; GMP, 1000; PreBNK, 800; MLP, 2000; MPP, 2000; transcript counts threshold used for the Lin−CD34/CD164 cells: Lin−CD164^{high}CD34^{low}Rep(Replicate)1, 1000; Lin−CD164^{high}CD34^{low}Rep2, 1000; Lin−CD164^{high}CD34-Rep1, 800; Lin−CD164^{high}CD34-Rep2, 800; Lin−CD164^{high}CD34+Rep1, 1000; Lin−CD164^{high}CD34+Rep2, 800; Lin−CD164^{low}CD34−, 700). Next, for all samples, the cells with >25% of their transcripts coming from mitochondrial genes were excluded as this is a marker of stressed or dying cells. The final number of barcodes used in the downstream analysis is summarized in Table 1. The gene expression counts of each cell were normalized using a total-count normalization variant that avoids distortion from very highly expressed genes, as described in Klein et al. Specifically, \grave{x}_{i,j}, the normalized transcript counts for gene j in cell i, was calculated from the raw counts x_{i,j} as follows: \grave{x}_{i,j}=x_{i,j}^{\grave{X}}/Xᵢ, in which Xᵢ=Σx_{i,j} and \grave{X} is the average of Xᵢ over all cells. To prevent very highly expressed genes from correspondingly decreasing the relative expression of other genes, genes comprising >5% of the total counts of any cell were excluded when calculating \grave{X} and Xᵢ.

Data Visualization and Construction of k-Nearest Neighbors Graphs

After filtering, the data were used to construct a k-nearest neighbor (kNN) graph, in which cells correspond to graph nodes and edges connect cells to their nearest neighbors. An independent kNN graph was generated for each dataset as follows. Genes were further filtered by selecting only genes with Fano factor (measure of dispersion) above a mean dependent threshold (median value) and requiring at least three UMIFM (Unique Molecular Identifiers Filtered Mapped) to be detected in at least three cells (sorted HSPC, n=5596 genes; sorted Lin−CD34/CD164 cells, n=7156 genes). Expression values for each gene were standardized independently by applying Z-score transformation. Unless otherwise stated, for all the analyses and graphical representations throughout the paper, z-scores have been used as a measure of gene activity. From previous experiments, it was observed that cell cycle and ribosomal associated genes can have a significant impact on the definition of cell clustering and on cell-to-cell transcriptional distance. For this reason, a G2/M genes set (UBE2C, HMGB2, HMGN2, TUBA1B, MKI67, CCNB1, TUBB, TOP2A, TUBB4B) and ribosomal genes set (RPL- and RPS-) was defined. A G2/M and a ribosomal signature score were then constructed by summing the average z-score of respective genes sets and removing genes that were highly correlated (Pearson r>0.2) with these signatures (sorted HSPC, n=117 genes; sorted Lin−CD34/CD164 cells, n=304 genes). Finally, dimensionality reduction by Principal Component Analysis (PCA) was performed. kNN graphs were constructed by setting k, number of neighbors, equal to 4, using the first 40 principal components and a Euclidean metric to measure distance between transcriptomes. The kNN graphs were visualized by means of a force-directed layout using the custom interactive software interface SPRING (Klein et al., Lab Chip 17, 2540-2541 (2017)) The final layout, corresponding to a minimal free energy configuration, showed a high degree of robustness with respect to different initialization (except for layout rotation that do not affect subsequent analyses). No manual adjustments were performed on the visualizations. Visual inspection on SPRING plot for Lin−CD34/CD164 transcriptome data set showed the presence of a cluster of cells (860 barcodes), highly interconnected and very poorly linked to the rest of the layout. Investigating for the presence of a particular gene expression signature characterizing this subpopulation, high levels of expression for mitochondrial genes (MT.CYB, MT.ATP6, MT.ND4, MT.ND1, MT.CO3, MT.ND3) was observed. These events had a peculiar transcriptional profile indicator of stressed or dying cells, which was not detected upon the dedicated filtering step, and were therefore manually removed from the final kNN graph.

Projection of Sorted HSPC Cells onto the Sorted Lin−CD34/CD164 Graph

To project subsets of sorted HSPC cells onto the Lin−CD34/CD164 graph, the intersection set among genes used to generate the two kNN graphs (n=5116 genes) was first identified. PCA was then performed on Lin−CD34/CD164 reduced expression matrix retaining the first 40 principal components. Sorted HSPC cells were projected on the Lin−CD34/CD164 principal component space upon z-score transformation of genes expression data with gene specific centering and scaling parameters derived from Lin−CD34/CD164 data. For each cell belonging to a specific group in the sorted HSPC data set (FACS sorted subpopulation or computationally identified group), the k=4 “most similar” cells in the Lin−CD34/CD164 map were identified using PCA scores and Euclidean distance. The graphical representations in FIG. 2-1F and FIG . 6, have been generated by rescaling the 2-dimensional Lin−CD34/CD164 SPRING layout to a unit squared area. Cell spatial distribution was calculated using a 2-dimensional kernel density estimator (bandwidths for x and y directions both set to 0.035) and using a contour plot for density level 1e-05 to highlight areas characterized by a non-negligible probability. A colored density estimation was overlaid (bandwidths for x and y directions both set to 0.1) for the spatial distribution of cells selected as most similar. The analysis and graphical representation allow for an intuitive understanding of in which area of the Lin−CD34/CD164 graph lie cells with a transcriptional configuration that better resemble HSPC subsets.

### Observed and Adjusted Cell Density Estimations

The transcriptional state related to small subpopulations such as the most primitive ones, are difficult to investigate by means of single-cell profiling on bulk heterogeneous populations. Introducing a fractionation strategy through FACS sorting before inDrops barcoding, this limitation was overcome by artificially over-representing primitive fractions inside the CD34+ and Lin− compartments. This aspect is shown in the 2-dimensional density estimation plotted in FIGS. 1-2A, 1-2B (left plots) where high density values can be found in graph areas associated to both primitive and committed cells. To provide a representation of what would have been instead the expected contribution of single cell events to the bulk human CD34+ and Lin− population, a weight was assigned to each cell, defined according to the proportion of events observed in the corresponding FACS gate. The graphs of FIGS. 1-2A and 1-2B (right), show densities obtained by keeping cells location constant and taking into account the calculated cell weights (Table 5).

### Table 5: Details for the Generation of the Observed and Predicted Cell Density Estimations Shown in FIGS. 1-2A and 1-2B.

TABLE 5
Details for the generation of the observed and predicted cell
density estimations shown in FIGS. 1-2A and 1-2B.

| | Post-filtering | FACS gating | Individual |
|---|---|---|---|
| Population | barcoded cells | proportion | barcode weight |
| HSC | 1282 | 0.132 | 1.03E−04 |
| MPP | 215 | 0.280 | 1.30E−03 |
| MLP | 123 | 0.044 | 3.55E−04 |
| PreB/NK | 592 | 0.019 | 3.26E−05 |
| MEP | 1211 | 0.006 | 4.96E−06 |
| CMP | 1576 | 0.325 | 2.07E−04 |
| GMP | 1012 | 0.193 | 1.91E−04 |
| Lin-CD34+CD164+ | 6343 | 0.074 | 1.30E−05 |
| Lin-CD34lowCD164high | 4266 | 0.073 | 1.78E−05 |
| Lin-CD34-CD164high | 4434 | 0.728 | 1.78E−04 |
| Lin-CD34-CD164low | 358 | 0.048 | 1.45E−04 |

### Transcriptional Principal Trajectories Identification

Both the topologies generated with SPRING reveal the presence of a continuum of transcriptional states connecting the most primitive subpopulations to more committed ones. Although some degree of variability is observed, layout topologies also suggest the presence of principal transcriptional trajectories during the differentiation process. The estimation and characterization of these trajectories could potentially allow: a) establish an order among transcriptional states with respect to differentiation process; b) group together cells with a common fate; c) investigate the gene regulatory dynamics underlying fate decision and lineages commitment. For these purposes, a procedure was implemented that composed the following main steps: 1) structure-aware filtering performed on transcriptome graph; 2) branching reconstruction by minimum spanning tree on reduced consolidating points; 3) association and ordering of cells according to inferred branching structure.

1) Structure-aware filtering. The structure-aware technique that was adopted is aimed at revealing and consolidating continuous, low-dimensional and high-density structures in the underlying higher-dimensional data, while ignoring noise and outliers. Briefly, its discretized version formulation, i.e. representing densities by sets of sample points, are described. Observed data points, pᵢ, are considered sampled from an underlying n-dimensional density fₚ(z), supposed to have been generated by adding noise to an underlying lower (min) m-dimensional data manifold. Consolidation points, xᵢ(t), are considered to be sampled from a time-dependent distribution fₓ(z,t), initialized as fₓ(z,0)=fₚ(z), that changes over time (iterations) guided by a time dependent velocity field that gradually remove noise while revealing the underlying m-dimensional structure in the input density fₚ(z). Initially, consolidation points can be either a random sample of data points or, as performed in this study, the whole data set. While data points are fixed in the n-dimensional manifold, the position of every consolidation point is iteratively updated according to the following formula:

$${{x}_{i} ( t + 1 )}^{′} = {x}_{i} ( t ) \frac{{\sum }_{j} {p}_{j} {K}^{′} ( \frac{1}{2} {‖ {p}_{j} - {x}_{i} ( t ) ‖}^{2} )}{{\sum }_{j} {K}^{′} ( \frac{1}{2} {‖ {p}_{j} - {x}_{i} ( t ) ‖}^{2} )} - μ \frac{\begin{matrix}{\sum }_{k} {A}^{′} A ( {x}_{k} ( t ) - {x}_{i} ( t ) ) \\ {L}^{′} ( \frac{1}{2} {‖ A ( {x}_{k} ( t ) - {x}_{i} ( t ) ) ‖}^{2} )\end{matrix}}{{\sum }_{k} {L}^{′} ( \frac{1}{2} {‖ A ( {x}_{k} ( t ) - {x}_{i} ( t ) ) ‖}^{2} )}$$

where K′ and L′ are first derivatives of a standard and modified Gaussian smoothing kernels defined as

$${K}^{′} ( \frac{1}{2} {‖ {p}_{j} - {x}_{i} ( t ) ‖}^{2} ) = {e}^{- ( {‖ {p}_{j} - {x}_{i} ( t ) ‖}^{2} ) / 2 {r}^{2}}$$

$$and$$

$${L}^{′} ( \frac{1}{2} {‖ A [ {x}_{k} ( t ) - {x}_{i} ( t ) ] ‖}^{2} ) = \frac{{e}^{- ( {‖ A [ {x}_{k} ( t ) - {x}_{i} ( t ) ] ‖}^{2} ) / 2 {r}^{2}}}{( {‖ A [ {x}_{k} ( t ) - {x}_{i} ( t ) ] ‖}^{2} )} ;$$

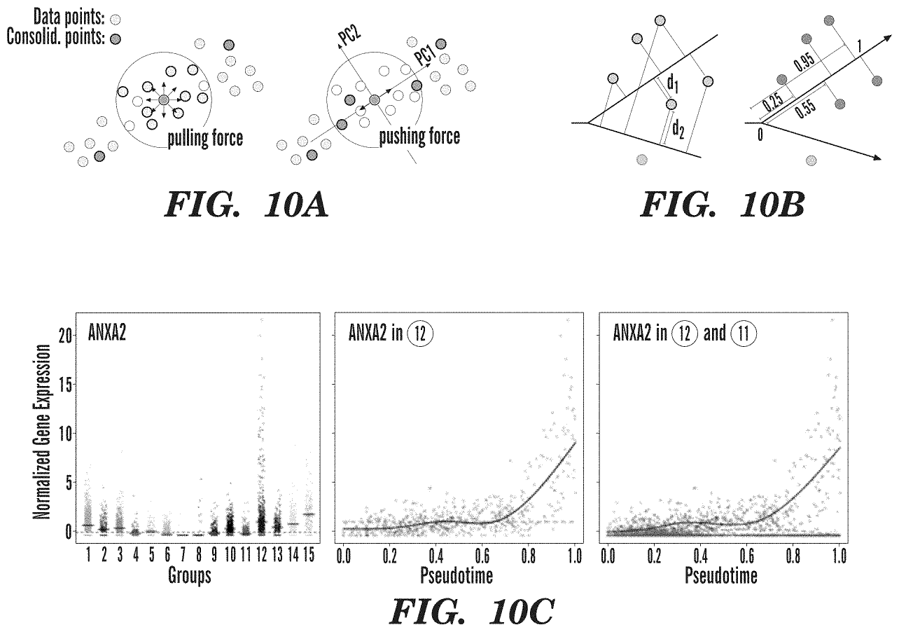

j and k mark respectively data and consolidation points within a radially symmetric, n-dimensional neighborhood of user-defined radius r centered on xᵢ(t); 0<μ<1 is a user-defined constant; Aᵢ=[λᵢ¹vᵢ¹;λᵢ²vᵢ²; . . . ;λᵢ^{m}vᵢ^{m}] is a n×m matrix with {λᵢ¹;λᵢ²; . . . ;λᵢ^{m}} and {vᵢ¹;vᵢ²; . . . ;vᵢ^{m}} respectively first m eigenvectors and eigenvalues of the kxn matrix in which the k-th row is equal to the n-dimensional vectors xₖ(t)−xᵢ(t) . The iterative procedure continues until the sum of consolidation points displacement, ΔX=Σ[xᵢ(t−1)−xᵢ(t)]² is greater than a given small ∈(0.001). In the updating formula it is possible to recognize two components: the first one, called the data term, “pulls” consolidation points toward local extrema (high-density regions) of the noisy input density. The second, called repulsion term, prevents clumping of consolidation points by “pushing” them along locally optimal directions, enhancing latent continuous m-dimensional structures. A graphical representation is given in FIG. 10A. In this work, structure-aware filtering was performed on the 2-dimensional representation of SPRING generated layouts, upon rescaling to the unit square 2-dimensional space as previously described. The goal was to highlight the underlying 1-dimensional (curves) representations (m=1). In general, given a value for radius size r, it returns an estimated optimal structure providing an accurate representation of data layout complexity and allowing for an interpretation in biological terms. Under the assumption of Gaussian distributed input data with known variance, this method estimates a lower-bound for r able to guarantee convergence to the true m-dimensional manifold. Algorithm input parameters complying with these indications were chosen, setting respectively for the sorted HSPC and Lin−CD34/CD164 cell graphs: r equal to 0.05 and 0.02; μ equal to 0.3 for both. To ensure reproducibility of the results, the set of consolidation points was initialized with the whole set of data points. In FIGS. 3-2A and 3-2B, initial, temporary (2nd, and 10th iterations) and the final configurations are shown.

2) Branching reconstruction by minimum spanning tree on reduced consolidating points. Structure-aware filtering returns coordinates of consolidation points in the n-dimensional input space such that they describe a continuum of locally optimal m-dimensional structures. In order to infer the principal transcriptional trajectories, the set of consolidation points was first reduced by iteratively averaging points closer than 0.01 (FIGS. 3-2A and 3-2B, Merging plots). This step has a regularization goal and allows for a considerable reduction of the data set size for downstream analyses. To connect points and design the graph skeleton, the minimum spanning tree algorithm was chosen, with Euclidean distance based edges weighting. Only in the sorted HSPC analysis was the small cluster located between Erythroid and Neuthrophils left unconnected due to its large distance from others consolidation points. The minimum spanning tree on reduced points is visible in FIGS. 3-2A and 3-2B, MST plots.

3) Branch association and cells ordering. Through the identification of bifurcation nodes, the minimum spanning tree was subdivided in segments (or trajectories, or branches) as shown in FIGS. 3-2A and 3-2B, Principal trajectories plots. Each cell has been associated to one segment, based on minimum distance criteria. In order to exclude cells with a transcriptional profile too different from those captured by the principal trajectories, cells more distant than 0.05 from any of the branches remained unlabeled. To order cells along the corresponding trajectory, the distance between the initial node (marked with 0 in FIG. 10B) and the projection of each cell onto the trajectory was calculated. Rescaled distances (0-1 interval), have been calculated and used as pseudo-time values in all gene expression analyses described herein.

### Generation of Diffusion Map

In order to verify the robustness of the results with respect to the adopted data analysis approach, the Lin−CD34/CD164 kNN-based transcriptome topology and inferred differentiation trajectories were compared to those derived from an alternative method, not relying on kNN, such as Diffusion map. R implementation of diffusion map available in package destiny, that is specifically designed for scRNA-seq data, was used. The matrix reporting the 40 principal components representation of filtered-and-normalized expression data, obtained as described in the Data visualization and construction of k-nearest neighbors graphs section, was passed as input argument to DiffusionMap function. All other DiffusionMap settings were kept to default configurations. The diffusion map for Lin−CD34/CD164 is shown in FIGS. 11A and 11B. The transcriptional principal trajectories identified starting from SPRING layout (FIG. 2-1D) were confirmed by applying the algorithm to the three-dimensional diffusion map. The results are reported in FIGS. 12A-12C.

### Gene Expression Analysis

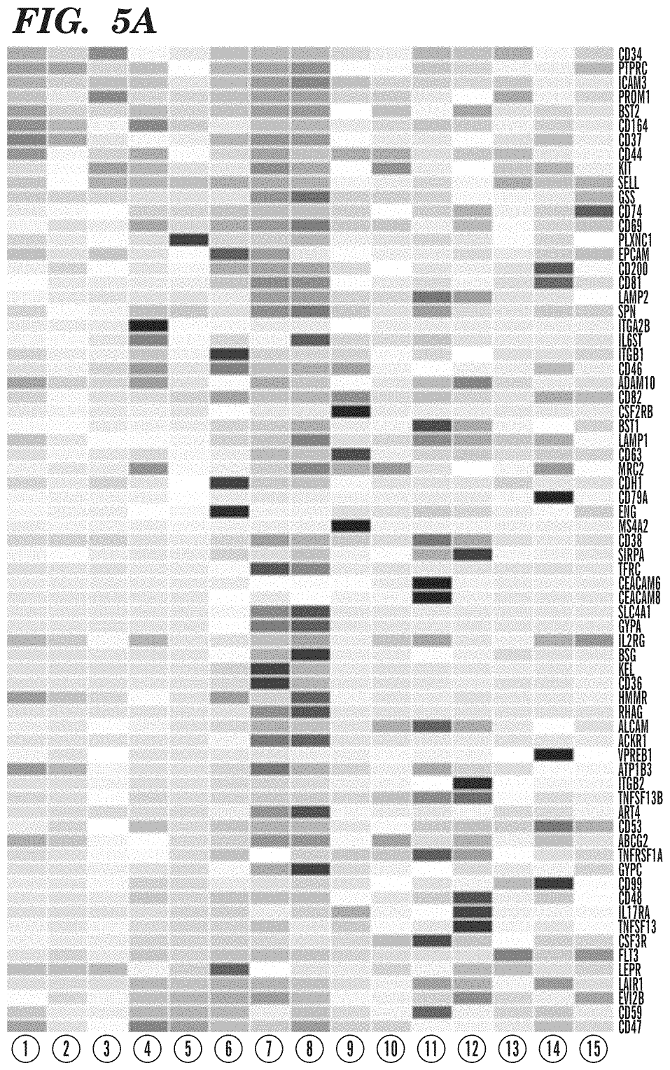

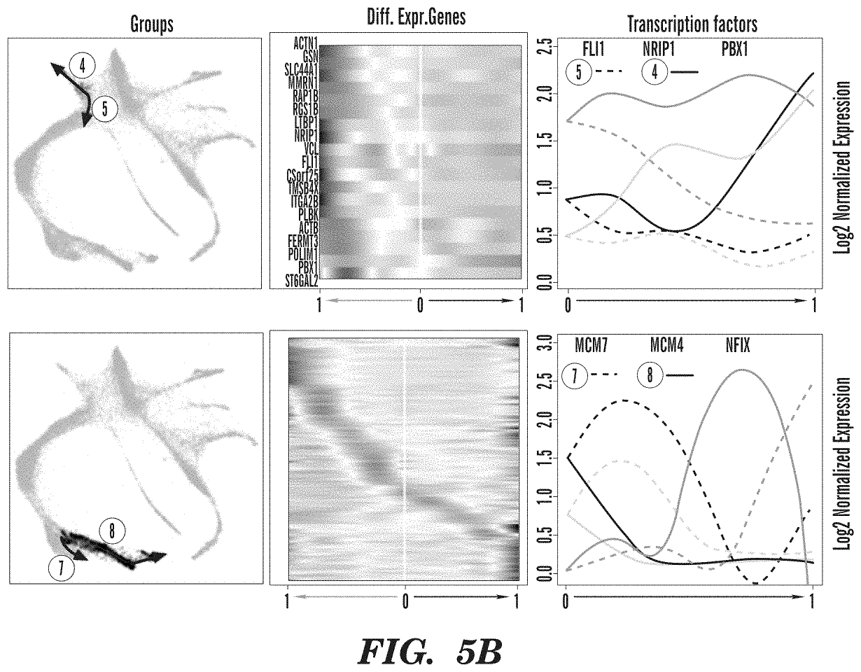

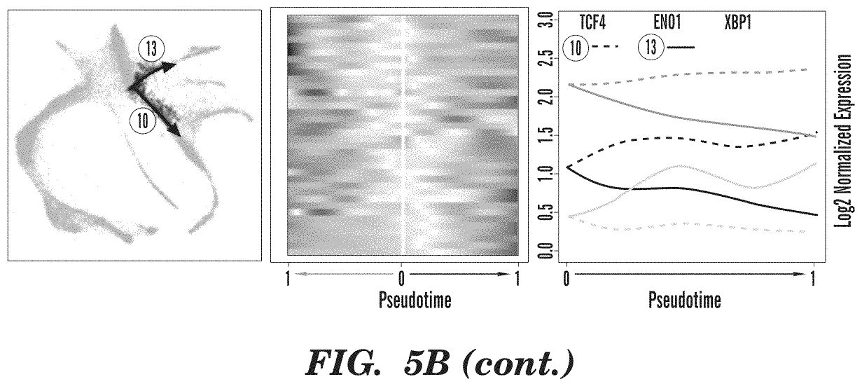

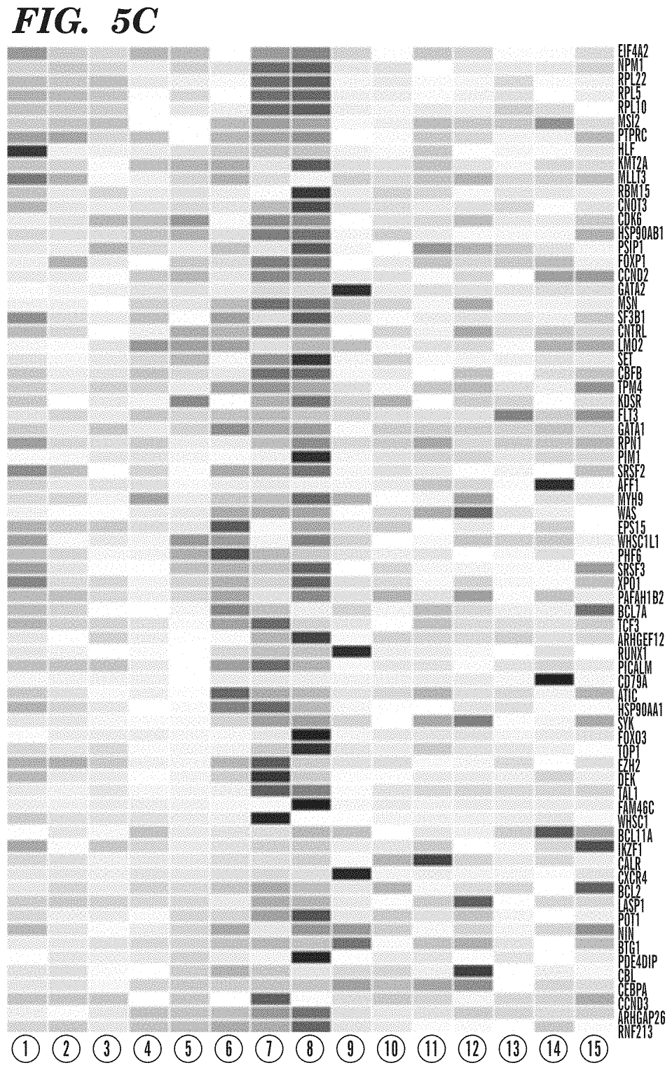

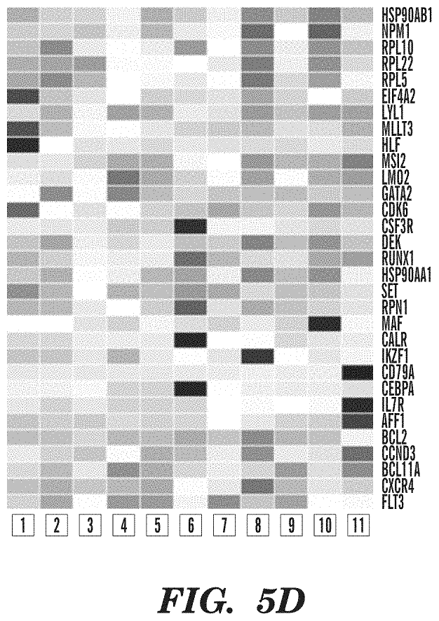

Throughout the manuscript different types of gene expression analysis have been shown. The statistical model underlying each of them has been defined according to the specific question of interest. The analyses can be grouped in the following categories with related examples shown in FIG. 10C) differentially expressed genes across cell groups (FIGS. 1-1C and 5A-5C); 2) identification of genes with a significant association between expression level and branch specific pseudotime ordering (FIGS. 3-1B, 3-1C, and 7); 3) investigation of differences in gene expression dynamics among trajectories (FIG. 2-1E; FIG. 5B). Similarly to what proposed in Trapnell et al., a Generalized Additive Models (GAM) approach was used that allows to test the dependence between the response variable and different types of predictors in a more flexible manner, for example, by estimating regression coefficients by using different loss functions (M-estimators) or by modelling trend with nonparametric functions. To prevent the potentially high impact of expression value outliers and dropouts, frequently observed in single cell RNA-Seq data, in all fitting procedures the Huber loss function for regression was used. Huber loss function is commonly used in robust regression and consists in a piecewise penalty function in which a quadratic penalization is replaced by a linear one for large differences. Its tuning constant has been set to k=0.862, meaning that the linear loss is applied to differences below the 10^{th} and above 90^{th} percentile, assuming a central Gaussian part of the distribution of residuals.

Differentially expressed genes across cell groups have been identified by fitting and comparing the two following models for each gene separately. The full model assumes gene expression averages to be group-dependent. From a practical view-point, model likelihood and coefficients have been calculated by using group labels Gᵢ,i=1, . . . ,k, where k is the number of groups, as dummy variables, M₁:μ(Y)=β₀+β₁G₁+ . . . +βₖGₖ, where μ(Y) is the average expression value for a gene. The restricted (null) model M₀:μ(Y)=β₀ instead, assumes no-differences in mean expression values among groups and considers variation only due to the intrinsic noise of expression measurements. Derived from this analysis are heatmaps in FIGS. 1-1G, 5A, and 5C where statistically significant genes within specific subsets (CD marker genes, Human and Mouse transcription factors, blood cancer associated proto-oncogenes) are shown. Detection of genes that significantly change as a function of pseudotime, t, has been done by comparing the likelihood of the model M₁:μ(Y,t)=β₀+s(t), where expression value trend μ(Y,t) varies according to a cubic splines (with four degree of freedom), s(t), to a flat null hypothesis M₀:μ(Y)=β₀ in which expression is assumed to randomly fluctuates around a constant value along the whole branch. All genes have been tested for association with respect to each branch. Panels in FIGS. 3-1B, 3-1C and FIG. 7 are based on this modelling approach. Finally, to find differences in gene expression dynamics underlying fate decisions and divergent differentiation trajectories, the following protocol can be used. As aforementioned, cell pseudo-time value can be interpreted as a measure of cell degree of maturation along a specific segment of the differentiation process. Even though it is difficult to make a direct comparison among the regulatory dynamics underlying commitment towards different lineages, by rescaling the branch total length to the unit interval, it is possible to test whether a gene behaves differently among branches. This is a simplistic approach that only partially takes into account the potential presence of different maturation paces or other confounding factors such as varying duplication/differentiation/death rates. In the formulation of the full model employed in this gene expression analysis, it was also assumed that cells belonging to trajectories stemming from a common bifurcation node, exhibit an expression pattern highly similar for pseudo-time values close to 0, that will then eventually progressively diverge toward more branch-specific transcriptional states. This assumption motivated the formulation of the model M₁:μ(Y,t)=β₀+sᵢ(t)Gᵢ+sⱼ(t)Gⱼ, in which branch-specific gene regression curves can evolve according to distinct pseudo-temporal dynamics sᵢ(t) and sⱼ(t), constrained to have the same expression value for t=0 (common intercept). The reduced model M₀:μ(Y,t)=β₀+s(t), allows gene expression average to vary over pseudotime according to a non-linear function, but assumes a common s(t) for both groups. In FIGS. 2-1E and 5B significant fate associated genes are reported. Transcription factors shown in FIGS. 2-1E and 5B have been selected (among those significant) because already proposed in the literature has correlated with lineage committed.

In all cases, the differences in explanatory power among M₁ and nested model M₀, have been tested by Chi-squared likelihood ratio test (LRT). All the analyses have been performed by means of custom R scripts available at GITHUB_REPOSITORY. For regression fitting and model testing, the VGAM library, and in particular vgam( ) huber1( ) and sm.bs( ), respectively was calculated for estimate, loss function and splines interpolation and lrtest( )for testing.

### Human-Mouse Erythropoiesis Comparisons

In order to compare the gene expression dynamics associated to human and mouse erythropoiesis, data generated by using inDrops technology on mouse Kit+ cells was used. For mouse data set, differentiation trajectories were identified and cells labeled (FIG. 3-1A) according to the methodology afore described. Subgroup 6 in mouse and subgroups 6, 7, 8 in human Lin−CD34/CD164 map were considered as representative of erythroid commitment (FIG. 3-1B top). Genes were tested for association to pseudotime in the two organisms separately (human: 3821; mouse: 1071 statistically significant genes, LRT adjusted p-value<0.05). Among those significant, 720 orthologous genes were retrieved based on Mouse Genome Database (MGD) (Mouse Genome Informatics website, The Jackson Laboratory, Bar Harbor, Me., informatics.jax.org), for which behavior is plotted by means of symmetric heatmap in FIG. 3-1B (bottom). Dissimilarities were further investigated by calculating Pearson correlations coefficients for each couple of human/mouse homologous genes (FIG. 7), and pathway enrichment analysis was performed using Reactome database on the 89 human genes exhibiting a low-or-negative correlation (Pearson correlation<0.5).

### Population Balance Analysis

To infer the structure of the hematopoietic lineage tree from the scRNA-seq data, PBA was applied and couplings between each pair of fates were calculated. For the HSPC subset dataset, PBA was run on the merged data, using the kNN graph constructed as above (“Data visualization and construction of k-nearest neighbors graphs”). PBA was run as described in (Tusi et al.). Briefly, negative values of R were assigned (the local imbalance between cell division and cell loss) to the five cells with highest gene signature score for each fate (see next paragraph) and a single positive value to the remaining cells such that

$${\sum }_{i} {R}_{i} = 0 .$$

Setting the diffusion constant to 1, the exit rates for each fate were fit such that the five cells with highest HSC signature had average fate probabilities within 1% of uniform. A similar procedure was carried out for the sorted Lin−CD34/CD164 dataset. Here, the analysis was restricted to the CD164^{high}CD34⁺ population, since this contained all uncommitted progenitors and the earliest uni-lineage progenitors. The kNN graph for PBA was constructed setting k to 40 to improve the robustness of the analysis, and the diffusion constant was set to 0.5 and used 10 cells per fate and 10 HSCs to fit the exit rates.

The following gene sets were used to define the lineage-specific signatures for the HSPC subset dataset:
Meg: ITGA2B, PF4, VWF
E: CA1, HBB, KLF1, TFR2
DC: CCR2, IRF8, MPEG1
G: ELANE, MPO, LYZ, CSF1R, CTSG, PRTN3, AZU1
Ly1: RGS1, NPTX2, DDIT4, ID2
Ly2: DNTT, RAG1, RAG2
HSC: CRHBP, HLF, DUSP1
   And for the Lin−CD34/CD164 dataset:
E: KLF1, CA1
Meg: ITGA2B, PLEK
BEM: CLC, CPA3, HDC
Ly: DNTT, CD79A, VPREB1
DC: IRF8, SPIB, IGKC
M: LYZ, MS4A6A, ANXA2
N: ELANE
HSC: HLF, ADGRG6, CRHBP, PCDH9

For both datasets, the PBA-predicted fate probabilities were used to infer a differentiation hierarchy, as described in (Tusi et al. Nature 555, 54-60 (2018)) (FIGS. 1-1F, 2-1C). A fate coupling score (see next paragraph) was computed for each pair of fates, and pairs with scores significantly higher than expected under the null model were joined and their fate probabilities merged by addition. This process was carried out iteratively until all fates were joined.

The coupling score between two fates A and B is the number of cells with P(A)P(B)>ε, using ε=1/14 throughout. A null distribution was generated for each pair of fates by computing the coupling scores for 1000 permutations of the original fate probabilities, re-normalizing each cell's probabilities at each randomization. The significance of the observed couplings was measured using the z-score with respect to the null distribution.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for obtaining an enriched population comprising primitive hematopoietic stem/progenitor cells for use in transplantation or gene therapy, the method comprising selecting one or more CD34⁺CD164^{high} cells, and expanding said cells in culture to enrich for stem/progenitor cells.

2. (canceled)

3. The method of claim 1, wherein the CD34⁺CD164^{high} selection enriches for stem/progenitor cells at greater than about 60% 70%, 80% or 90% efficiency.

4. The method of claim 1, further comprising characterizing the expanded population for the presence of early stage progenitor cells by detecting increased levels of CD164 versus the level of CD164 present in a late progenitor cell.

5. The method of claim 1, wherein there is an order of magnitude difference in the level of CD164 present in an early stage progenitor cell versus the level present in a late progenitor cell.

6. The method of claim 1, wherein the level of CD164 in an early stage progenitor is at least about 10³ to 10⁴, whereas the level of CD164 is about 10² in a late stage progenitor cell.

7. (canceled)

8. The method of claim 1, wherein the method excludes B cell progenitors.

9. The method of claim 8, wherein the method excludes B cell progenitors expressing CD79a and/or CD10.

10. A method for selecting early versus late hematopoietic stem/progenitor cells, the method comprising isolating CD34⁺CD164^{high} cells from CD34⁺CD164^{low} cells.

11. The method of claim 1, wherein the selecting comprises contacting the cell with a CD34 antibody and a CD164 antibody.

12-14. (canceled)

15. The method of claim 1, further comprising characterizing the cells for one or more markers selected from the group consisting of CD3, CD7, CD10, CD14, CD16, CD15, CD19, CD20, CD38, CD41, CD45RA, CD56, CD71, CD90, CD135, and Lin.

16. A method for obtaining an enriched population comprising primitive hematopoietic stem/progenitor cells for use in transplantation or gene therapy, the method comprising
(a) selecting one or more CD34⁺CD164^{high} cells;
(b) expanding said cells in culture to obtain a population of stem cells; and
(c) selecting CD34⁺CD164^{high} cells from the population of step (b), thereby obtaining a population of primitive hematopoietic stem/progenitor cells.

17. (canceled)

18. A cell or population of cells obtained according to the method of claim 1.

19. (canceled)

20. A method for treating a subject in need of an increase in hematopoietic stem/progenitor cells, the method comprising administering to the subject an effective amount of a cell of claim 18 present in a pharmaceutically acceptable excipient.

21. A method for expressing a therapeutic gene in a hematopoietic cell of a subject, comprising:
(a) contacting a hematopoietic stem/progenitor cell with a recombinant vector comprising a nucleic acid sequence encoding a therapeutic or detectable polypeptide to obtain a transgenic cell transduced with the vector; and
(b) administering the cell to a subject, such that the transgenic cell or a progeny cell thereof populates bone marrow in the subject and expresses the therapeutic or detectable polypeptide.

22-26. (canceled)

27. The method of claim 21, wherein the subject has a condition selected from the group consisting of lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation, immune thrombocytopenic purpura, myelodysplasia; thrombocytotic disease, thrombocytosis, congenital neutropenias, myelodysplastic syndrome; and neutropenia associated with chemotherapy and/or radiotherapy.

28. (canceled)

29. A method to support short-term granulopoiesis in conditioned neutropenic patients, the method comprising administering to the subject an effective amount of a cell of claim 18 present in a pharmaceutically acceptable excipient.

30. A method for sustaining early phase, late phase, or early and late phases of hematopoietic reconstitution comprising administering to a subject an effect amount of a cell of claim 18 present in a pharmaceutically acceptable excipient.

31. A pharmaceutical composition comprising an effective amount of a cell of claim 18.

32. A kit for treating a subject in need of an increase in hematopoietic stem/progenitor cells, the kit comprising a cell of claim 18 and instructions for administering the cell to a subject.

33. A method for obtaining an enriched population comprising cells having basophilic potential for use in transplantation or gene therapy, the method comprising
selecting one or more Lin−CD34+CD135− cells, and expanding said cells in culture to enrich for cells having basophilic potential; or
(a) selecting one or more having basophilic potential cells;
(b) expanding said cells in culture to obtain a population of stem cells; and
(c) selecting Lin—CD34+CD135− cells from the population of step (b), thereby obtaining a population of cells having basophilic potential.

34-35. (canceled)

36. A cell or population of cells obtained according to the method of claim 33.

37. A method for treating a subject in need of an increase in basophils, the method comprising administering to the subject an effective amount of a cell or population of cells of claim 36 present in a pharmaceutically acceptable excipient.
